(19) United States
(12) Patent Application Publication
(10) Pub. No.: US 2014/0161786 A1
(43) Pub. Date: Jun. 12, 2014

(54) Therapeutic compositions and related methods

(71) Applicants: Jonathan B. Rothbard, Sonoma, CA (US); Lawrence Steinman, Stanford, CA (US); Michael P. Kurnellas, Palo Alto, CA (US)

(72) Inventors: Jonathan B. Rothbard, Sonoma, CA (US); Lawrence Steinman, Stanford, CA (US); Michael P. Kurnellas, Palo Alto, CA (US)

(21) Appl. No.: 13/987,770

(22) Filed: Aug. 29, 2013

Related U.S. Application Data
(60) Provisional application No. 61/743,477, filed on Sep. 4, 2012.

(51) Int. Cl. C07K 7/06; A61K 38/08; A61K 45/06

(52) U.S. Cl. CPC C07K 7/06; A61K 45/06; A61K 38/08
USPC 424/94.6; 514/21.8; 514/15.3; 514/15.2; 514/14.7; 514/13.7

(57) ABSTRACT

The present invention generally relates to therapeutic compositions for the treatment of mammalian disease and related methods. It more specifically relates to compositions comprising peptides for the treatment of inflammation and methods for delivering the peptides. In one composition aspect, the composition comprises a peptide, and the peptide comprises one or more hexapeptides that are capable of forming fibrils.

### CROSS REFERENCE TO RELATED APPLICATIONS

This application claims the benefit of priority of U.S. Provisional Patent Application No. 61/743,477 filed Sep. 4, 2012, which is incorporated herein by reference in its entirety.

### STATEMENT REGARDING FEDERALLY SPONSORED RESEARCH OR DEVELOPMENT

This invention was made with Government support under contract NS055997 awarded by the National Institutes of Health. The Government has certain rights in this invention.

### REFERENCE TO SEQUENCE LISTING

The present application is being filed along with a Sequence Listing in electronic format. The Sequence Listing is provided as a file entitled 2013-08-28 ST-001.02 ST_{—}25.txt, created Aug. 29, 2013, which is 176 KB in size. The information in the electronic format of the Sequence Listing is incorporated herein by reference in its entirety. The content of the sequence listing information recorded in computer readable form is identical to the written sequence listing.

### FIELD OF THE INVENTION

The present invention generally relates to therapeutic compositions for the treatment of mammalian diseases and related methods. It more specifically relates to compositions comprising peptides for the treatment of inflammation and methods for delivering the peptides.

### BACKGROUND OF THE INVENTION

Accumulation of amyloid fibrils correlates with many disease states. Such disease states include: Alzheimer's disease; diabetes mellitus type 2; Parkinson's disease; transmissible spongiform encephalopathy; Huntington's disease; medullary carcinoma of the thyroid; cardiac arrhythmias; atherosclerosis; rheumatoid arthritis; aortic medical amyloid; prolactinomas; familial amyloid polyneuropathy; hereditary non-neuropathic systemic amyloidosis; dialysis related amyloidosis; Finnish amyloidosis; lattice corneal dystrophy; cerebral amyloid angiopathy; systemic AL amyloidosis; and, sporadic inclusion body myositis.

The reason for a correlation between amyloid fibril accumulation and the specified diseases is unclear. Amyloid deposits physically disrupt tissue architecture in certain cases, which may impair function by a bulk process. Fibril deposition is associated with mitochondrial dysfunction, and the resulting initiation of apoptosis, in other cases. There have been some reports indicating that amyloid polymers can induce polymerization of essential amyloidogenic proteins, which is detrimental to cells.

### SUMMARY OF THE INVENTION

In a composition aspect, the present invention provides a composition for treatment of one or more inflammatory conditions in a mammal. The composition comprises a peptide comprising amino acids of L-configuration, D-configuration or a mixture of configurations, wherein the peptide comprises one or more hexapeptides of a structure that generates a free energy of formation of −23 kcal/mol or less (i.e., more negative) when the hexapeptide sequence in subjected to the Rosetta-Profile algorithm. Example structures are provided below.

In another composition aspect, the present invention provides a composition for treatment of one or more inflammatory conditions in a mammal. The composition comprises a fibril. The fibril comprises four or more hexapeptides comprising amino acids of an L-configuration, D-configuration or a mixture of configurations having free energy of formation of −23 kcal/mol or less (i.e., more negative) when the hexapeptide sequence in subjected to the Rosetta-Profile algorithm.

In another composition aspect, the present invention provides a composition for treatment of one or more inflammatory conditions in a mammal. The composition comprises a fibril and a plasma protein. The fibril comprises four or more hexapeptides comprising amino acids of an L-configuration, D-configuration or a mixture of configurations having free energy of formation of −23 kcal/mol or less (i.e., more negative) when the hexapeptide sequence in subjected to the Rosetta-Profile algorithm.

In a method aspect, the present invention provides a method of treating inflammation comprising the step of administering one of the compositions discussed above.

### BRIEF DESCRIPTION OF THE FIGURES

FIG. 1 shows graphs related to the therapeutic effectiveness of amyloidogenic hexamers in a mouse model.

FIG. 2 shows bar graphs related to the modulation of plasma cytokine levels by certain amyloidogenic hexamers.

FIG. 3 shows bar graphs related to the pH dependency of amyloid formulation for certain amyloidogenic hexamers.

FIG. 4 shows graphs related to the correlation of ThT staining of amyloid fibrils with molecular chaperone function.

FIG. 5 shows bar graphs related to the binding of plasma proteins by amyloid fibrils.

FIG. 6 shows various graphs related to the comparison of L- and D-amino acids with respect to formation of amyloid fibrils and effect as molecular chaperones having therapeutic efficiencies.

### DETAILED DESCRIPTION OF THE INVENTION

The present invention generally relates to therapeutic compositions for the treatment of mammalian disease and related methods. It more specifically relates to compositions comprising peptides for the treatment of inflammation and methods for delivering the peptides.

“Complex” refers to a molecular entity formed by non-covalent association involving two or more component molecular entities (ionic or uncharged). The bonding between the components is normally weaker than in a covalent bond. Typically, the dissociation constant for a complex (“K_{d}”) is equal to or greater than 1 μM. In certain cases, it is equal to or greater than “Fibril” refers to an aggregation of four or more peptides of the present invention. In some cases, the aggregate includes one hundred or more, one thousand or more, five thousand or more, or ten thousand or more peptides.

In a composition aspect, the present invention is directed to one or more peptides that exhibit a therapeutic effect when administered to a mammal, typically an anti-inflammatory effect. The one or more peptides comprise, or consist of, hexapeptides comprising amino acids of an L-configuration, D-configuration or a mixture of configurations that generate a free energy of formation of −23 kcal/mol or less (i.e., more negative) when the hexapeptide sequence in subjected to the Rosetta-Profile algorithm. In certain cases, the peptide is a hexapeptide. The peptides have a carboxy terminus and an amino terminus.

In certain cases the peptides of the present invention comprise 1, 2, or 3 amino acids having polar basic side chains. The polar basic side chains may have a terminal amino or a terminal imidazole group. In other cases, the peptides comprise 1 polar acidic side chain. In other cases, the peptides comprise 0, 1, 2, 3, 4, or 5 amino acids having hydrophobic side chains. In still other cases, the peptides comprise 0, 1, 2, 3, 4, or 5 amino acids having polar uncharged side chains, wherein the peptide has a positive charge.

The carboxy terminus of the peptides is typically either a carboxylic acid (i.e., —CO₂H) or an amide (i.e., —C(O)NR₂, where R is a substituent such as alkyl or hydrogen). The amino terminus is typically either an amine (i.e., N(R′)₂, where R′ is a substituent such as alkyl or hydrogen) or an acetate group (i.e., —C(O)R″, where R″ is a substituent such as methyl, ethyl, or longer alkyl).

The one or more peptides may comprise, or consist of, one or more of the following hexapeptides, where each indicated amino acid is either an L-amino acid or a D-amino acid (symbol for hexapeptide indicated in parenthesis after the hexamer):

| | |
|---|---|
| SVNVDL; | (SEQ ID NO: 1) |
| SLNVDV; | (SEQ ID NO: 2) |
| SVDVNL; | (SEQ ID NO: 3) |
| DLSVVL; | (SEQ ID NO: 4) |
| SVNLDV; | (SEQ ID NO: 5) |
| SVVNDV; | (SEQ ID NO: 6) |
| DVSLVN; | (SEQ ID NO: 7) |
| DVSVLN; | (SEQ ID NO: 8) |
| SDLVNV; | (SEQ ID NO: 9) |
| SLNVVS; | (SEQ ID NO: 10) |
| LNVDSV; | (SEQ ID NO: 11) |
| NDLSVV; | (SEQ ID NO: 12) |
| VDNLVS; | (SEQ ID NO: 13) |
| VNDVSL; | (SEQ ID NO: 14) |
| VSDNVL; | (SEQ ID NO: 15) |
| LNDVVS; | (SEQ ID NO: 16) |
| LSVDVN; | (SEQ ID NO: 17) |
| NSVDLV; | (SEQ ID NO: 18) |
| VDVLNS; | (SEQ ID NO: 19) |
| VNDSVL; | (SEQ ID NO: 20) |
| VNVSLD; | (SEQ ID NO: 21) |
| LDVNSV; | (SEQ ID NO: 22) |
| LNVVDS; | (SEQ ID NO: 23) |
| LSDVVN; | (SEQ ID NO: 24) |
| LVVNDS; | (SEQ ID NO: 25) |
| NSLDVV; | (SEQ ID NO: 26) |
| VLVDNS; | (SEQ ID NO: 27) |
| VNLSDV; | (SEQ ID NO: 28) |
| VNVDLS; | (SEQ ID NO: 29) |
| DNVSVD; | (SEQ ID NO: 30) |
| LSDNVV; | (SEQ ID NO: 31) |
| LSVVDN; | (SEQ ID NO: 32) |
| VLDVSN; | (SEQ ID NO: 33) |
| VNDLVS; | (SEQ ID NO: 34) |
| VNVSDL; | (SEQ ID NO: 35) |
| VVLSDN; | (SEQ ID NO: 36) |
| LVSVNL; | (SEQ ID NO: 37) |
| LVNVSV; | (SEQ ID NO: 38) |
| SLNVSV; | (SEQ ID NO: 39) |
| LVSVNS; | (SEQ ID NO: 40) |
| SVDVNV; | (SEQ ID NO: 41) |
| LVVSVL; | (SEQ ID NO: 42) |
| VNLVVS; | (SEQ ID NO: 43) |
| SNLVSV; | (SEQ ID NO: 44) |
| SVNVLS; | (SEQ ID NO: 45) |
| VLVSVL; | (SEQ ID NO: 46) |
| LVNVSL; | (SEQ ID NO: 47) |
| SVNVDS; | (SEQ ID NO: 48) |
| VLSVNV; | (SEQ ID NO: 49) |
| NLVVSV; | (SEQ ID NO: 50) |
| SVVLNV; | (SEQ ID NO: 51) |
| LSVVNL; | (SEQ ID NO: 52) |
| SNLVVS; | (SEQ ID NO: 53) |
| SVVLDV; | (SEQ ID NO: 54) |
| LVSLNV; | (SEQ ID NO: 55) |
| VSLNVV; | (SEQ ID NO: 56) |
| VKVQIY; | (SEQ ID NO: 57) |
| QVVIYK; | (SEQ ID NO: 58) |
| KVIQVY; | (SEQ ID NO: 59) |
| VYVKIY; | (SEQ ID NO: 60) |
| QIVVYK; | (SEQ ID NO: 61) |
| QVIKVY; | (SEQ ID NO: 62) |
| VQVKIY; | (SEQ ID NO: 63) |
| QVVKIY; | (SEQ ID NO: 64) |
| QVIVYK; | (SEQ ID NO: 65) |
| QIVKVY; | (SEQ ID NO: 66) |
| QKIVVY; | (SEQ ID NO: 67) |
| QKVVYI; | (SEQ ID NO: 68) |
| KVQVYI; | (SEQ ID NO: 69) |
| QVVKYI; | (SEQ ID NO: 70) |
| KVQIYV; | (SEQ ID NO: 71) |
| VKIQVY; | (SEQ ID NO: 72) |
| VIQKVY; | (SEQ ID NO: 73) |
| KVVIYK; | (SEQ ID NO: 74) |
| QIVKYV; | (SEQ ID NO: 75) |
| QKVIYV; | (SEQ ID NO: 76) |
| KQVVIY; | (SEQ ID NO: 77) |
| KIQVYV; | (SEQ ID NO: 78) |
| KVYVQI; | (SEQ ID NO: 79) |
| VVQIYK; | (SEQ ID NO: 80) |
| KQVIVY; | (SEQ ID NO: 81) |
| VQIKVY; | (SEQ ID NO: 82) |
| QKIVYV; | (SEQ ID NO: 83) |
| VIQVYK; | (SEQ ID NO: 84) |
| KVVIQY; | (SEQ ID NO: 85) |
| KVQVIY; | (SEQ ID NO: 86) |
| QYVVIK; | (SEQ ID NO: 87) |
| YVVIQK; | (SEQ ID NO: 88) |
| KIVVQY; | (SEQ ID NO: 89) |
| YQVIVK; | (SEQ ID NO: 90) |
| KIYVQV; | (SEQ ID NO: 91) |
| KVVIYQ; | (SEQ ID NO: 92) |
| KYVVQI; | (SEQ ID NO: 93) |
| YQIVVK; | (SEQ ID NO: 94) |
| YVVIQY; | (SEQ ID NO: 95) |
| KQVIYV; | (SEQ ID NO: 96) |
| KYVQVI; | (SEQ ID NO: 97) |
| KYVVIQ; | (SEQ ID NO: 98) |
| QKVVIY; | (SEQ ID NO: 99) |
| QYVIVK; | (SEQ ID NO: 100) |
| IVQKVY; | (SEQ ID NO: 101) |
| KYIVVQ; | (SEQ ID NO: 102) |
| QVIKYV; | (SEQ ID NO: 103) |
| YIVVQK; | (SEQ ID NO: 104) |
| YQVVIK; | (SEQ ID NO: 105) |
| IKVQVY; | (SEQ ID NO: 106) |
| KVVQYI; | (SEQ ID NO: 107) |
| KYVQIV; | (SEQ ID NO: 108) |
| VKQVYI; | (SEQ ID NO: 109) |
| VQVIYK; | (SEQ ID NO: 110) |
| VVIQKY; | (SEQ ID NO: 111) |
| IQVVYK; | (SEQ ID NO: 112) |
| KIVVYQ; | (SEQ ID NO: 113) |
| KYQVIV; | (SEQ ID NO: 114) |
| KYVIVQ; | (SEQ ID NO: 115) |
| VVYIQK; | (SEQ ID NO: 116) |
| IVQVYK; | (SEQ ID NO: 117) |
| KQIVYV; | (SEQ ID NO: 118) |
| KYVIQV; | (SEQ ID NO: 119) |
| QKVIVY; | (SEQ ID NO: 120) |
| QYIKVV; | (SEQ ID NO: 121) |
| QYIVKV; | (SEQ ID NO: 122) |
| VQIVYK; | (SEQ ID NO: 123) |
| VVQKYI; | (SEQ ID NO: 124) |
| VYQIVK; | (SEQ ID NO: 125) |
| YVQVIK; | (SEQ ID NO: 126) |
| QIVYVK; | (SEQ ID NO: 127) |
| IKVYQV; | (SEQ ID NO: 128) |
| KVVYIQ; | (SEQ ID NO: 129) |
| VQKYIV; | (SEQ ID NO: 130) |
| VQKYVI; | (SEQ ID NO: 131) |
| KVVQIY; | (SEQ ID NO: 132) |
| YVIKIY; | (SEQ ID NO: 133) |
| QVIVYV; | (SEQ ID NO: 134) |
| VVIKVY; | (SEQ ID NO: 135) |
| KVVIYI; | (SEQ ID NO: 136) |
| VYVVIK; | (SEQ ID NO: 137) |
| YIVQVY; | (SEQ ID NO: 138) |
| YVIQVY; | (SEQ ID NO: 139) |
| YVIVVY; | (SEQ ID NO: 140) |
| KVIVYI; | (SEQ ID NO: 141) |
| YVIKVY; | (SEQ ID NO: 142) |
| VQVIVK; | (SEQ ID NO: 143) |
| YVVKIY; | (SEQ ID NO: 144) |
| YVVQVI; | (SEQ ID NO: 145) |
| QIVVYQ; | (SEQ ID NO: 146) |
| VVQIVK; | (SEQ ID NO: 147) |
| VQIVVK; | (SEQ ID NO: 148) |
| VIVVYK; | (SEQ ID NO: 149) |
| IVQVYI; | (SEQ ID NO: 150) |
| YVVIQV; | (SEQ ID NO: 151) |
| VYQVVI; | (SEQ ID NO: 152) |
| KYIQVY; | (SEQ ID NO: 153) |
| KYVVIY; | (SEQ ID NO: 154) |
| KYVQIY; | (SEQ ID NO: 155) |
| VVQVIK; | (SEQ ID NO: 156) |
| QKIVVK; | (SEQ ID NO: 157) |
| KYVIVY; | (SEQ ID NO: 158) |
| KQVVIK; | (SEQ ID NO: 159) |
| YVQIYV; | (SEQ ID NO: 160) |
| YKVQVY; | (SEQ ID NO: 161) |
| IKVQIY; | (SEQ ID NO: 162) |
| KIVVQK; | (SEQ ID NO: 163) |
| VIKVVI; | (SEQ ID NO: 164) |
| VYIKVV; | (SEQ ID NO: 165) |
| VYIVQV; | (SEQ ID NO: 166) |
| IVYVQI; | (SEQ ID NO: 167) |
| KVQIYK; | (SEQ ID NO: 168) |
| IYVIVY; | (SEQ ID NO: 169) |
| VIYVIV; | (SEQ ID NO: 170) |
| KYQVYI; | (SEQ ID NO: 171) |
| VIQKVV; | (SEQ ID NO: 172) |
| VKIVYV; | (SEQ ID NO: 173) |
| VKQIVV; | (SEQ ID NO: 174) |
| YIVKQY; | (SEQ ID NO: 175) |
| YIVVQY; | (SEQ ID NO: 176) |
| YKIQVY; | (SEQ ID NO: 177) |
| YQVVIY; | (SEQ ID NO: 178) |
| YVVKQY; | (SEQ ID NO: 179) |
| VIVKVQ; | (SEQ ID NO: 180) |
| QKVVIK; | (SEQ ID NO: 181) |
| GMVVVG; | (SEQ ID NO: 182) |
| GVVVMG; | (SEQ ID NO: 183) |
| GVVMVG; | (SEQ ID NO: 184) |
| GGVVVM; | (SEQ ID NO: 185) |
| GVVGVM; | (SEQ ID NO: 186) |
| GVVVGM; | (SEQ ID NO: 187) |
| GVMVVG; | (SEQ ID NO: 188) |
| GGVVMV; | (SEQ ID NO: 189) |
| GGVMVV; | (SEQ ID NO: 190) |
| GMVVGV; | (SEQ ID NO: 191) |
| GVVMGV; | (SEQ ID NO: 192) |
| GMVGVV; | (SEQ ID NO: 193) |
| GVMVGV; | (SEQ ID NO: 194) |
| GVVGMV; | (SEQ ID NO: 195) |
| GGMVVV; | (SEQ ID NO: 196) |
| GVGVMV; | (SEQ ID NO: 197) |
| GVGVVM; | (SEQ ID NO: 198) |
| MVVVGM; | (SEQ ID NO: 199) |
| GMGVVV; | (SEQ ID NO: 200) |
| MVVVGG; | (SEQ ID NO: 201) |
| GVGMVV; | (SEQ ID NO: 202) |
| GVVGGV; | (SEQ ID NO: 203) |
| MVGVVG; | (SEQ ID NO: 204) |
| VVMVGG; | (SEQ ID NO: 205) |
| MGVVVG; | (SEQ ID NO: 206) |
| MVGVGV; | (SEQ ID NO: 207) |
| VGVMVG; | (SEQ ID NO: 208) |
| MVVGVG; | (SEQ ID NO: 209) |
| VVGVMG; | (SEQ ID NO: 210) |
| VMGVVG; | (SEQ ID NO: 211) |
| VGVGVM; | (SEQ ID NO: 212) |
| VMVGVG; | (SEQ ID NO: 213) |
| MGGVVV; | (SEQ ID NO: 214) |
| VGMVVG; | (SEQ ID NO: 215) |
| VVGVGM; | (SEQ ID NO: 216) |
| VGGVVM; | (SEQ ID NO: 217) |
| VVGMVG; | (SEQ ID NO: 218) |
| VGVVMG; | (SEQ ID NO: 219) |
| MVGGVV; | (SEQ ID NO: 220) |
| VGMVGV; | (SEQ ID NO: 221) |
| VMVVGG; | (SEQ ID NO: 222) |
| VVGGVM; | (SEQ ID NO: 223) |
| VGGVMV; | (SEQ ID NO: 224) |
| VVVGMG; | (SEQ ID NO: 225) |
| VVGMGV; | (SEQ ID NO: 226) |
| MGVGVV; | (SEQ ID NO: 227) |
| VGVVGM; | (SEQ ID NO: 228) |
| VGGMVV; | (SEQ ID NO: 229) |
| VVVMGG; | (SEQ ID NO: 230) |
| VVMGGV; | (SEQ ID NO: 231) |
| VVVGGM; | (SEQ ID NO: 232) |
| VVGGMV; | (SEQ ID NO: 233) |
| VGMGVV; | (SEQ ID NO: 234) |
| VMGGVV; | (SEQ ID NO: 235) |
| VGGVGV; | (SEQ ID NO: 236) |
| VGVMGV; | (SEQ ID NO: 237) |
| VGVGMV; | (SEQ ID NO: 238) |
| and | |
| VMVGGV; | (SEQ ID NO: 239) |
| ANSTSV; | (SEQ ID NO: 240) |
| ANSVSG; | (SEQ ID NO: 241) |
| ANSVSS; | (SEQ ID NO: 242) |
| AQNSNV; | (SEQ ID NO: 243) |
| AQNVNS; | (SEQ ID NO: 244) |
| AQNVTS; | (SEQ ID NO: 245) |
| AQSQSV; | (SEQ ID NO: 246) |
| AQSSSV; | (SEQ ID NO: 247) |
| AQSTSV; | (SEQ ID NO: 248) |
| AQSVNS; | (SEQ ID NO: 249) |
| AQSVQS; | (SEQ ID NO: 250) |
| AQSVSQ; | (SEQ ID NO: 251) |
| AQSVSS; | (SEQ ID NO: 252) |
| AQSVST; | (SEQ ID NO: 253) |
| ASNNNV; | (SEQ ID NO: 254) |
| ASNQNQ; | (SEQ ID NO: 255) |
| ASNQNV; | (SEQ ID NO: 256) |
| ASNQTQ; | (SEQ ID NO: 257) |
| ASNSNV; | (SEQ ID NO: 258) |
| ASNSTV; | (SEQ ID NO: 259) |
| ASNTNS; | (SEQ ID NO: 260) |
| ASNTNV; | (SEQ ID NO: 261) |
| ASNTSV; | (SEQ ID NO: 262) |
| ASNVNG; | (SEQ ID NO: 263) |
| ASNVNQ; | (SEQ ID NO: 264) |
| ASNVNS; | (SEQ ID NO: 265) |
| ASNVNT; | (SEQ ID NO: 266) |
| ASNVTG; | (SEQ ID NO: 267) |
| ASNVTT; | (SEQ ID NO: 268) |
| ASSNSV; | (SEQ ID NO: 269) |
| ASSVSG; | (SEQ ID NO: 270) |
| ASSVSN; | (SEQ ID NO: 271) |
| ATNVNS; | (SEQ ID NO: 272) |
| ATNVTS; | (SEQ ID NO: 273) |
| ATSQSQ; | (SEQ ID NO: 274) |
| ATSQSV; | (SEQ ID NO: 275) |
| ATSTSG; | (SEQ ID NO: 276) |
| ATSTSV; | (SEQ ID NO: 277) |
| ATSVSG; | (SEQ ID NO: 278) |
| ATSVSS; | (SEQ ID NO: 279) |
| AVNQNS; | (SEQ ID NO: 280) |
| AVNQSQ; | (SEQ ID NO: 281) |
| AVNSNG; | (SEQ ID NO: 282) |
| AVNSNS; | (SEQ ID NO: 283) |
| AVNSNT; | (SEQ ID NO: 284) |
| AVNTNS; | (SEQ ID NO: 285) |
| AVSNSG; | (SEQ ID NO: 286) |
| AVSNSS; | (SEQ ID NO: 287) |
| AVSQNQ; | (SEQ ID NO: 288) |
| AVSQSG; | (SEQ ID NO: 289) |
| AVSQSQ; | (SEQ ID NO: 290) |
| AVSQTQ; | (SEQ ID NO: 291) |
| AVSSNQ; | (SEQ ID NO: 292) |
| AVSSNS; | (SEQ ID NO: 293) |
| AVSSSQ; | (SEQ ID NO: 294) |
| AVSTSG; | (SEQ ID NO: 295) |
| GANTVS; | (SEQ ID NO: 296) |
| GAQTSS; | (SEQ ID NO: 297) |
| GASNQS; | (SEQ ID NO: 298) |
| GASQQS; | (SEQ ID NO: 299) |
| GASSQQ; | (SEQ ID NO: 300) |
| GGQVTS; | (SEQ ID NO: 301) |
| GGSNQV; | (SEQ ID NO: 302) |
| GNNVQS; | (SEQ ID NO: 303) |
| GNQVTS; | (SEQ ID NO: 304) |
| GNSNQV; | (SEQ ID NO: 305) |
| GNSQQQ; | (SEQ ID NO: 306) |
| GNSQQS; | (SEQ ID NO: 307) |
| GNSQQV; | (SEQ ID NO: 308) |
| GNSSTV; | (SEQ ID NO: 309) |
| GNSTQS; | (SEQ ID NO: 310) |
| GNSTQV; | (SEQ ID NO: 311) |
| GNSTVS; | (SEQ ID NO: 312) |
| GNSVQS; | (SEQ ID NO: 313) |
| GNSVSS; | (SEQ ID NO: 314) |
| GNSVST; | (SEQ ID NO: 315) |
| GNSVTS; | (SEQ ID NO: 316) |
| GQNTVS; | (SEQ ID NO: 317) |
| GQNVAS; | (SEQ ID NO: 318) |
| GQNVQS; | (SEQ ID NO: 319) |
| GQNVSS; | (SEQ ID NO: 320) |
| GQNVTS; | (SEQ ID NO: 321) |
| GQQQSQ; | (SEQ ID NO: 322) |
| GQQTSS; | (SEQ ID NO: 323) |
| GQQTSV; | (SEQ ID NO: 324) |
| GQQVAS; | (SEQ ID NO: 325) |
| GQQVNS; | (SEQ ID NO: 326) |
| GQQVQS; | (SEQ ID NO: 327) |
| GQQVSG; | (SEQ ID NO: 328) |
| GQQVSQ; | (SEQ ID NO: 329) |
| GQQVSS; | (SEQ ID NO: 330) |
| GQQVST; | (SEQ ID NO: 331) |
| GQQVTS; | (SEQ ID NO: 332) |
| GQSGQV; | (SEQ ID NO: 333) |
| GQSNQA; | (SEQ ID NO: 334) |
| GQSNQS; | (SEQ ID NO: 335) |
| GQSNQV; | (SEQ ID NO: 336) |
| GQSQAQ; | (SEQ ID NO: 337) |
| GQSQQQ; | (SEQ ID NO: 338) |
| GQSQQS; | (SEQ ID NO: 339) |
| GQSQSQ; | (SEQ ID NO: 340) |
| GQSSQQ; | (SEQ ID NO: 341) |
| GQSSQS; | (SEQ ID NO: 342) |
| GQSSQV; | (SEQ ID NO: 343) |
| GQSTQS; | (SEQ ID NO: 344) |
| GQSTQV; | (SEQ ID NO: 345) |
| GQSVAG; | (SEQ ID NO: 346) |
| GQSVAQ; | (SEQ ID NO: 347) |
| GQSVAS; | (SEQ ID NO: 348) |
| GQSVQN; | (SEQ ID NO: 349) |
| GQSVQQ; | (SEQ ID NO: 350) |
| GQSVQS; | (SEQ ID NO: 351) |
| GQSVSG; | (SEQ ID NO: 352) |
| GQSVSN; | (SEQ ID NO: 353) |
| GQSVSQ; | (SEQ ID NO: 354) |
| GQSVSS; | (SEQ ID NO: 355) |
| GQSVST; | (SEQ ID NO: 356) |
| GQSVTS; | (SEQ ID NO: 357) |
| GSNQQV; | (SEQ ID NO: 358) |
| GSNQVQ; | (SEQ ID NO: 359) |
| GSNSTV; | (SEQ ID NO: 360) |
| GSNSVQ; | (SEQ ID NO: 361) |
| GSNSVT; | (SEQ ID NO: 362) |
| GSNTAV; | (SEQ ID NO: 363) |
| GSNTQV; | (SEQ ID NO: 364) |
| GSNTVA; | (SEQ ID NO: 365) |
| GSNTVS; | (SEQ ID NO: 366) |
| GSNVAS; | (SEQ ID NO: 367) |
| GSNVQQ; | (SEQ ID NO: 368) |
| GSNVQS; | (SEQ ID NO: 369) |
| GSNVQT; | (SEQ ID NO: 370) |
| GSNVTS; | (SEQ ID NO: 371) |
| GSQQSV; | (SEQ ID NO: 372) |
| GSQQTQ; | (SEQ ID NO: 373) |
| GSQQTV; | (SEQ ID NO: 374) |
| GSQTSS; | (SEQ ID NO: 375) |
| GSQTSV; | (SEQ ID NO: 376) |
| GSQVAS; | (SEQ ID NO: 377) |
| GSQVNS; | (SEQ ID NO: 378) |
| GSQVQS; | (SEQ ID NO: 379) |
| GSQVSS; | (SEQ ID NO: 380) |
| GSQVST; | (SEQ ID NO: 381) |
| GSQVTG; | (SEQ ID NO: 382) |
| GSQVTS; | (SEQ ID NO: 383) |
| GSSAQS; | (SEQ ID NO: 384) |
| GSSGQV; | (SEQ ID NO: 385) |
| GSSNQA; | (SEQ ID NO: 386) |
| GSSNQT; | (SEQ ID NO: 387) |
| GSSNQV; | (SEQ ID NO: 388) |
| GSSNSV; | (SEQ ID NO: 389) |
| GSSNTV; | (SEQ ID NO: 390) |
| GSSNVT; | (SEQ ID NO: 391) |
| GSSQAQ; | (SEQ ID NO: 392) |
| GSSQQA; | (SEQ ID NO: 393) |
| GSSQQV; | (SEQ ID NO: 394) |
| GSSQSQ; | (SEQ ID NO: 395) |
| GSSSQQ; | (SEQ ID NO: 396) |
| GSSSQS; | (SEQ ID NO: 397) |
| GSSSQV; | (SEQ ID NO: 398) |
| GSSTQA; | (SEQ ID NO: 399) |
| GSSTQG; | (SEQ ID NO: 400) |
| GSSTQT; | (SEQ ID NO: 401) |
| GSSTQV; | (SEQ ID NO: 402) |
| GSSTSV; | (SEQ ID NO: 403) |
| GSSTVN; | (SEQ ID NO: 404) |
| GSSVNS; | (SEQ ID NO: 405) |
| GSSVQN; | (SEQ ID NO: 406) |
| GSSVQT; | (SEQ ID NO: 407) |
| GSSVSG; | (SEQ ID NO: 408) |
| GSSVSS; | (SEQ ID NO: 409) |
| GSSVST; | (SEQ ID NO: 410) |
| GSSVTN; | (SEQ ID NO: 411) |
| GTNSSV; | (SEQ ID NO: 412) |
| GTNSVS; | (SEQ ID NO: 413) |
| GTNTVS; | (SEQ ID NO: 414) |
| GTNVQS; | (SEQ ID NO: 415) |
| GTNVSS; | (SEQ ID NO: 416) |
| GTNVTS; | (SEQ ID NO: 417) |
| GTQQSQ; | (SEQ ID NO: 418) |
| GTQSTS; | (SEQ ID NO: 419) |
| GTQSTV; | (SEQ ID NO: 420) |
| GTQTSA; | (SEQ ID NO: 421) |
| GTQTSV; | (SEQ ID NO: 422) |
| GTQVQS; | (SEQ ID NO: 423) |
| GTQVSG; | (SEQ ID NO: 424) |
| GTQVSN; | (SEQ ID NO: 425) |
| GTQVSS; | (SEQ ID NO: 426) |
| GTQVST; | (SEQ ID NO: 427) |
| GTSAQS; | (SEQ ID NO: 428) |
| GTSGQV; | (SEQ ID NO: 429) |
| GTSNQA; | (SEQ ID NO: 430) |
| GTSNQS; | (SEQ ID NO: 431) |
| GTSNQT; | (SEQ ID NO: 432) |
| GTSNQV; | (SEQ ID NO: 433) |
| GTSNSV; | (SEQ ID NO: 434) |
| GTSNVS; | (SEQ ID NO: 435) |
| GTSQQA; | (SEQ ID NO: 436) |
| GTSQQS; | (SEQ ID NO: 437) |
| GTSQQV; | (SEQ ID NO: 438) |
| GTSQSQ; | (SEQ ID NO: 439) |
| GTSQSV; | (SEQ ID NO: 440) |
| GTSSNV; | (SEQ ID NO: 441) |
| GTSTQA; | (SEQ ID NO: 442) |
| GTSTQS; | (SEQ ID NO: 443) |
| GTSTQT; | (SEQ ID NO: 444) |
| GTSTQV; | (SEQ ID NO: 445) |
| GTSTSV; | (SEQ ID NO: 446) |
| GTSVAG; | (SEQ ID NO: 447) |
| GTSVAS; | (SEQ ID NO: 448) |
| GTSVNS; | (SEQ ID NO: 449) |
| GTSVNT; | (SEQ ID NO: 450) |
| GTSVQG; | (SEQ ID NO: 451) |
| GTSVQN; | (SEQ ID NO: 452) |
| GTSVQQ; | (SEQ ID NO: 453) |
| GTSVQS; | (SEQ ID NO: 454) |
| GTSVQT; | (SEQ ID NO: 455) |
| GTSVSG; | (SEQ ID NO: 456) |
| GTSVSN; | (SEQ ID NO: 457) |
| GTSVSS; | (SEQ ID NO: 458) |
| GTSVST; | (SEQ ID NO: 459) |
| GTSVTS; | (SEQ ID NO: 460) |
| GVNSQS; | (SEQ ID NO: 461) |
| GVNSST; | (SEQ ID NO: 462) |
| GVNSTS; | (SEQ ID NO: 463) |
| GVNTQS; | (SEQ ID NO: 464) |
| GVNTSS; | (SEQ ID NO: 465) |
| GVQNTS; | (SEQ ID NO: 466) |
| GVQQSQ; | (SEQ ID NO: 467) |
| GVQSNQ; | (SEQ ID NO: 468) |
| GVQSNS; | (SEQ ID NO: 469) |
| GVQSSG; | (SEQ ID NO: 470) |
| GVQSSQ; | (SEQ ID NO: 471) |
| GVQSTS; | (SEQ ID NO: 472) |
| GVQSTT; | (SEQ ID NO: 473) |
| GVQTNS; | (SEQ ID NO: 474) |
| GVQTQS; | (SEQ ID NO: 475) |
| GVQTSG; | (SEQ ID NO: 476) |
| GVQTSS; | (SEQ ID NO: 477) |
| GVSGQG; | (SEQ ID NO: 478) |
| GVSGQT; | (SEQ ID NO: 479) |
| GVSNAS; | (SEQ ID NO: 480) |
| GVSNNV; | (SEQ ID NO: 481) |
| GVSNQG; | (SEQ ID NO: 482) |
| GVSNQN; | (SEQ ID NO: 483) |
| GVSNQQ; | (SEQ ID NO: 484) |
| GVSNQS; | (SEQ ID NO: 485) |
| GVSNQT; | (SEQ ID NO: 486) |
| GVSNSS; | (SEQ ID NO: 487) |
| GVSNST; | (SEQ ID NO: 488) |
| GVSNTS; | (SEQ ID NO: 489) |
| GVSQAQ; | (SEQ ID NO: 490) |
| GVSQNQ; | (SEQ ID NO: 491) |
| GVSQNS; | (SEQ ID NO: 492) |
| GVSQQG; | (SEQ ID NO: 493) |
| GVSQQS; | (SEQ ID NO: 494) |
| GVSQQT; | (SEQ ID NO: 495) |
| GVSQSG; | (SEQ ID NO: 496) |
| GVSQSQ; | (SEQ ID NO: 497) |
| GVSQSS; | (SEQ ID NO: 498) |
| GVSSAQ; | (SEQ ID NO: 499) |
| GVSSAS; | (SEQ ID NO: 500) |
| GVSSGQ; | (SEQ ID NO: 501) |
| GVSSNG; | (SEQ ID NO: 502) |
| GVSSNQ; | (SEQ ID NO: 503) |
| GVSSNS; | (SEQ ID NO: 504) |
| GVSSNT; | (SEQ ID NO: 505) |
| GVSSQG; | (SEQ ID NO: 506) |
| GVSSQN; | (SEQ ID NO: 507) |
| GVSSQQ; | (SEQ ID NO: 508) |
| GVSSSG; | (SEQ ID NO: 509) |
| GVSSSQ; | (SEQ ID NO: 510) |
| GVSSSS; | (SEQ ID NO: 511) |
| GVSSTN; | (SEQ ID NO: 512) |
| GVSTAS; | (SEQ ID NO: 513) |
| GVSTNS; | (SEQ ID NO: 514) |
| GVSTQG; | (SEQ ID NO: 515) |
| GVSTQN; | (SEQ ID NO: 516) |
| GVSTQQ; | (SEQ ID NO: 517) |
| GVSTQS; | (SEQ ID NO: 518) |
| GVSTQT; | (SEQ ID NO: 519) |
| GVSTSN; | (SEQ ID NO: 520) |
| GVSTSQ; | (SEQ ID NO: 521) |
| GVSTSS; | (SEQ ID NO: 522) |
| GVSTST; | (SEQ ID NO: 523) |
| GVTNSS; | (SEQ ID NO: 524) |
| GVTSNS; | (SEQ ID NO: 525) |
| GVTSSN; | (SEQ ID NO: 526) |
| NGSTSV; | (SEQ ID NO: 527) |
| NGSVTS; | (SEQ ID NO: 528) |
| NNSVSS; | (SEQ ID NO: 529) |
| NQSQSQ; | (SEQ ID NO: 530) |
| NQSVSN; | (SEQ ID NO: 531) |
| NQSVSQ; | (SEQ ID NO: 532) |
| NQSVSS; | (SEQ ID NO: 533) |
| NSSNSV; | (SEQ ID NO: 534) |
| NSSQSQ; | (SEQ ID NO: 535) |
| NSSTVG; | (SEQ ID NO: 536) |
| NSSVSG; | (SEQ ID NO: 537) |
| NSSVSN; | (SEQ ID NO: 538) |
| NSSVSS; | (SEQ ID NO: 539) |
| NSSVTG; | (SEQ ID NO: 540) |
| NTNVNS; | (SEQ ID NO: 541) |
| NTQVSS; | (SEQ ID NO: 542) |
| NTSGSV; | (SEQ ID NO: 543) |
| NTSQSQ; | (SEQ ID NO: 544) |
| NTSTSS; | (SEQ ID NO: 545) |
| NTSTSV; | (SEQ ID NO: 546) |
| NTSVSS; | (SEQ ID NO: 547) |
| NVSGST; | (SEQ ID NO: 548) |
| NVSNSG; | (SEQ ID NO: 549) |
| NVSQSG; | (SEQ ID NO: 550) |
| NVSQSQ; | (SEQ ID NO: 551) |
| NVSQSS; | (SEQ ID NO: 552) |
| NVSSSG; | (SEQ ID NO: 553) |
| NVSSSS; | (SEQ ID NO: 554) |
| NVSSTG; | (SEQ ID NO: 555) |
| NVSTSN; | (SEQ ID NO: 556) |
| NVSTSS; | (SEQ ID NO: 557) |
| NVSTST; | (SEQ ID NO: 558) |
| QNSTSV; | (SEQ ID NO: 559) |
| QNSVSG; | (SEQ ID NO: 560) |
| QQSQSQ; | (SEQ ID NO: 561) |
| QQSVAQ; | (SEQ ID NO: 562) |
| QQSVAQ; | (SEQ ID NO: 563) |
| QQSVAS; | (SEQ ID NO: 564) |
| QQSVAT; | (SEQ ID NO: 565) |
| QQSVSG; | (SEQ ID NO: 566) |
| QQSVSN; | (SEQ ID NO: 567) |
| QQSVSS; | (SEQ ID NO: 568) |
| QSNQVQ; | (SEQ ID NO: 569) |
| QSNTAV; | (SEQ ID NO: 570) |
| QSNTNV; | (SEQ ID NO: 571) |
| QSSNSV; | (SEQ ID NO: 572) |
| QSSQAQ; | (SEQ ID NO: 573) |
| QSSQSQ; | (SEQ ID NO: 574) |
| QSSQSV; | (SEQ ID NO: 575) |
| QSSTSV; | (SEQ ID NO: 576) |
| QSSVSG; | (SEQ ID NO: 577) |
| QSSVSN; | (SEQ ID NO: 578) |
| QSSVSQ; | (SEQ ID NO: 579) |
| QSSVSS; | (SEQ ID NO: 580) |
| QSSVTG; | (SEQ ID NO: 581) |
| QSSVTS; | (SEQ ID NO: 582) |
| QTSQSQ; | (SEQ ID NO: 583) |
| QTSSSQ; | (SEQ ID NO: 584) |
| QTSSSV; | (SEQ ID NO: 585) |
| QTSVAG; | (SEQ ID NO: 586) |
| QTSVAS; | (SEQ ID NO: 587) |
| QTSVSG; | (SEQ ID NO: 588) |
| QTSVSN; | (SEQ ID NO: 589) |
| QTSVSS; | (SEQ ID NO: 590) |
| QTSVST; | (SEQ ID NO: 591) |
| QTSVTS; | (SEQ ID NO: 592) |
| QVSNSG; | (SEQ ID NO: 593) |
| QVSNSS; | (SEQ ID NO: 594) |
| QVSNST; | (SEQ ID NO: 595) |
| QVSQAQ; | (SEQ ID NO: 596) |
| QVSQTG; | (SEQ ID NO: 597) |
| QVSQTQ; | (SEQ ID NO: 598) |
| QVSSAQ; | (SEQ ID NO: 599) |
| QVSSSG; | (SEQ ID NO: 600) |
| QVSSSQ; | (SEQ ID NO: 601) |
| QVSSSS; | (SEQ ID NO: 602) |
| QVSSTQ; | (SEQ ID NO: 603) |
| QVSTSG; | (SEQ ID NO: 604) |
| QVSTSN; | (SEQ ID NO: 605) |
| QVSTSS; | (SEQ ID NO: 606) |
| QVSTST; | (SEQ ID NO: 607) |
| SAQQSQ; | (SEQ ID NO: 608) |
| SAQQTQ; | (SEQ ID NO: 609) |
| SASQQQ; | (SEQ ID NO: 610) |
| SASQSQ; | (SEQ ID NO: 611) |
| SGNSTV; | (SEQ ID NO: 612) |
| SGNTSV; | (SEQ ID NO: 613) |
| SGNVST; | (SEQ ID NO: 614) |
| SGNVTS; | (SEQ ID NO: 615) |
| SGQQTQ; | (SEQ ID NO: 616) |
| SGQQTV; | (SEQ ID NO: 617) |
| SGQTSV; | (SEQ ID NO: 618) |
| SGQVSS; | (SEQ ID NO: 619) |
| SGQVTG; | (SEQ ID NO: 620) |
| SGQVTQ; | (SEQ ID NO: 621) |
| SGQVTT; | (SEQ ID NO: 622) |
| SGSNTV; | (SEQ ID NO: 623) |
| SGSTNV; | (SEQ ID NO: 624) |
| SGSTQV; | (SEQ ID NO: 625) |
| SGSVAS; | (SEQ ID NO: 626) |
| SGSVNT; | (SEQ ID NO: 627) |
| SGSVQG; | (SEQ ID NO: 628) |
| SGSVQS; | (SEQ ID NO: 629) |
| SNNQTV; | (SEQ ID NO: 630) |
| SNNTAV; | (SEQ ID NO: 631) |
| SNNTNV; | (SEQ ID NO: 632) |
| SNNTQV; | (SEQ ID NO: 633) |
| SNQQSQ; | (SEQ ID NO: 634) |
| SNQQSV; | (SEQ ID NO: 635) |
| SNQQTQ; | (SEQ ID NO: 636) |
| SNQQTV; | (SEQ ID NO: 637) |
| SNQSTV; | (SEQ ID NO: 638) |
| SNQTSV; | (SEQ ID NO: 639) |
| SNQVSG; | (SEQ ID NO: 640) |
| SNQVSQ; | (SEQ ID NO: 641) |
| SNQVSS; | (SEQ ID NO: 642) |
| SNQVST; | (SEQ ID NO: 643) |
| SNQVTG; | (SEQ ID NO: 644) |
| SNQVTQ; | (SEQ ID NO: 645) |
| SNQVTS; | (SEQ ID NO: 646) |
| SNSGAV; | (SEQ ID NO: 647) |
| SNSNAV; | (SEQ ID NO: 648) |
| SNSNGV; | (SEQ ID NO: 649) |
| SNSNQS; | (SEQ ID NO: 650) |
| SNSQAG; | (SEQ ID NO: 651) |
| SNSQAQ; | (SEQ ID NO: 652) |
| SNSQAS; | (SEQ ID NO: 653) |
| SNSQAT; | (SEQ ID NO: 654) |
| SNSQAV; | (SEQ ID NO: 655) |
| SNSQGQ; | (SEQ ID NO: 656) |
| SNSQGV; | (SEQ ID NO: 657) |
| SNSQQA; | (SEQ ID NO: 658) |
| SNSQQG; | (SEQ ID NO: 659) |
| SNSQQQ; | (SEQ ID NO: 660) |
| SNSQQS; | (SEQ ID NO: 661) |
| SNSQQT; | (SEQ ID NO: 662) |
| SNSQQV; | (SEQ ID NO: 663) |
| SNSQSG; | (SEQ ID NO: 664) |
| SNSQST; | (SEQ ID NO: 665) |
| SNSQSV; | (SEQ ID NO: 666) |
| SNSSAV; | (SEQ ID NO: 667) |
| SNSSGV; | (SEQ ID NO: 668) |
| SNSSQG; | (SEQ ID NO: 669) |
| SNSSQQ; | (SEQ ID NO: 670) |
| SNSSQT; | (SEQ ID NO: 671) |
| SNSSQV; | (SEQ ID NO: 672) |
| SNSSSQ; | (SEQ ID NO: 673) |
| SNSSSV; | (SEQ ID NO: 674) |
| SNSTAG; | (SEQ ID NO: 675) |
| SNSTGS; | (SEQ ID NO: 676) |
| SNSTNV; | (SEQ ID NO: 677) |
| SNSTQA; | (SEQ ID NO: 678) |
| SNSTQQ; | (SEQ ID NO: 679) |
| SNSTQS; | (SEQ ID NO: 680) |
| SNSTQV; | (SEQ ID NO: 681) |
| SNSTSA; | (SEQ ID NO: 682) |
| SNSTSV; | (SEQ ID NO: 683) |
| SNSTVG; | (SEQ ID NO: 684) |
| SNSVAQ; | (SEQ ID NO: 685) |
| SNSVAS; | (SEQ ID NO: 686) |
| SNSVAT; | (SEQ ID NO: 687) |
| SNSVGQ; | (SEQ ID NO: 688) |
| SNSVGS; | (SEQ ID NO: 689) |
| SNSVGT; | (SEQ ID NO: 690) |
| SNSVNS; | (SEQ ID NO: 691) |
| SNSVNT; | (SEQ ID NO: 692) |
| SNSVQQ; | (SEQ ID NO: 693) |
| SNSVQS; | (SEQ ID NO: 694) |
| SNSVSG; | (SEQ ID NO: 695) |
| SNSVSQ; | (SEQ ID NO: 696) |
| SNSVSS; | (SEQ ID NO: 697) |
| SNSVST; | (SEQ ID NO: 698) |
| SNSVTG; | (SEQ ID NO: 699) |
| SQNVAS; | (SEQ ID NO: 700) |
| SQNVNS; | (SEQ ID NO: 701) |
| SQQQSQ; | (SEQ ID NO: 702) |
| SQQQSV; | (SEQ ID NO: 703) |
| SQQQTQ; | (SEQ ID NO: 704) |
| SQQQTV; | (SEQ ID NO: 705) |
| SQQSTV; | (SEQ ID NO: 706) |
| SQQVSG; | (SEQ ID NO: 707) |
| SQQVSN; | (SEQ ID NO: 708) |
| SQQVSS; | (SEQ ID NO: 709) |
| SQQVST; | (SEQ ID NO: 710) |
| SQQVTG; | (SEQ ID NO: 711) |
| SQQVTN; | (SEQ ID NO: 712) |
| SQQVTS; | (SEQ ID NO: 713) |
| SQQVTT; | (SEQ ID NO: 714) |
| SQSNAS; | (SEQ ID NO: 715) |
| SQSNAV; | (SEQ ID NO: 716) |
| SQSNGV; | (SEQ ID NO: 717) |
| SQSNQT; | (SEQ ID NO: 718) |
| SQSNQV; | (SEQ ID NO: 719) |
| SQSNSV; | (SEQ ID NO: 720) |
| SQSQAQ; | (SEQ ID NO: 721) |
| SQSQAS; | (SEQ ID NO: 722) |
| SQSQAV; | (SEQ ID NO: 723) |
| SQSQQQ; | (SEQ ID NO: 724) |
| SQSQQV; | (SEQ ID NO: 725) |
| SQSQSQ; | (SEQ ID NO: 726) |
| SQSSAV; | (SEQ ID NO: 727) |
| SQSSGV; | (SEQ ID NO: 728) |
| SQSSQG; | (SEQ ID NO: 729) |
| SQSSQV; | (SEQ ID NO: 730) |
| SQSSSQ; | (SEQ ID NO: 731) |
| SQSSSV; | (SEQ ID NO: 732) |
| SQSTAV; | (SEQ ID NO: 733) |
| SQSTQS; | (SEQ ID NO: 734) |
| SQSTSG; | (SEQ ID NO: 735) |
| SQSTSV; | (SEQ ID NO: 736) |
| SQSVAG; | (SEQ ID NO: 737) |
| SQSVAN; | (SEQ ID NO: 738) |
| SQSVAQ; | (SEQ ID NO: 739) |
| SQSVAS; | (SEQ ID NO: 740) |
| SQSVAT; | (SEQ ID NO: 741) |
| SQSVGG; | (SEQ ID NO: 742) |
| SQSVGN; | (SEQ ID NO: 743) |
| SQSVGQ; | (SEQ ID NO: 744) |
| SQSVGS; | (SEQ ID NO: 745) |
| SQSVGT; | (SEQ ID NO: 746) |
| SQSVNG; | (SEQ ID NO: 747) |
| SQSVNS; | (SEQ ID NO: 748) |
| SQSVQG; | (SEQ ID NO: 749) |
| SQSVQN; | (SEQ ID NO: 750) |
| SQSVQQ; | (SEQ ID NO: 751) |
| SQSVQS; | (SEQ ID NO: 752) |
| SQSVQT; | (SEQ ID NO: 753) |
| SQSVSG; | (SEQ ID NO: 754) |
| SQSVSN; | (SEQ ID NO: 755) |
| SQSVSS; | (SEQ ID NO: 756) |
| SQSVST; | (SEQ ID NO: 757) |
| SSNGTV; | (SEQ ID NO: 758) |
| SSNQNQ; | (SEQ ID NO: 759) |
| SSNQNV; | (SEQ ID NO: 760) |
| SSNQTQ; | (SEQ ID NO: 761) |
| SSNQTV; | (SEQ ID NO: 762) |
| SSNSTV; | (SEQ ID NO: 763) |
| SSNTNV; | (SEQ ID NO: 764) |
| SSNTQV; | (SEQ ID NO: 765) |
| SSNTVG; | (SEQ ID NO: 766) |
| SSNVAQ; | (SEQ ID NO: 767) |
| SSNVAS; | (SEQ ID NO: 768) |
| SSNVGT; | (SEQ ID NO: 769) |
| SSNVNG; | (SEQ ID NO: 770) |
| SSNVNQ; | (SEQ ID NO: 771) |
| SSNVNS; | (SEQ ID NO: 772) |
| SSNVNT; | (SEQ ID NO: 773) |
| SSNVQS; | (SEQ ID NO: 774) |
| SSNVQT; | (SEQ ID NO: 775) |
| SSNVTG; | (SEQ ID NO: 776) |
| SSNVTQ; | (SEQ ID NO: 777) |
| SSNVTS; | (SEQ ID NO: 778) |
| SSQNTV; | (SEQ ID NO: 779) |
| SSQQSV; | (SEQ ID NO: 780) |
| SSQQTQ; | (SEQ ID NO: 781) |
| SSQQTV; | (SEQ ID NO: 782) |
| SSQSTQ; | (SEQ ID NO: 783) |
| SSQTSV; | (SEQ ID NO: 784) |
| SSQVNS; | (SEQ ID NO: 785) |
| SSQVSN; | (SEQ ID NO: 786) |
| SSQVSQ; | (SEQ ID NO: 787) |
| SSQVSS; | (SEQ ID NO: 788) |
| SSQVST; | (SEQ ID NO: 789) |
| SSQVTG; | (SEQ ID NO: 790) |
| SSQVTQ; | (SEQ ID NO: 791) |
| SSQVTS; | (SEQ ID NO: 792) |
| SSQVTT; | (SEQ ID NO: 793) |
| SSSNAV; | (SEQ ID NO: 794) |
| SSSNQV; | (SEQ ID NO: 795) |
| SSSNSV; | (SEQ ID NO: 796) |
| SSSQAQ; | (SEQ ID NO: 797) |
| SSSQNQ; | (SEQ ID NO: 798) |
| SSSQQQ; | (SEQ ID NO: 799) |
| SSSQQT; | (SEQ ID NO: 800) |
| SSSQQV; | (SEQ ID NO: 801) |
| SSSQSQ; | (SEQ ID NO: 802) |
| SSSQSV; | (SEQ ID NO: 803) |
| SSSSGQ; | (SEQ ID NO: 804) |
| SSSSGV; | (SEQ ID NO: 805) |
| SSSSNV; | (SEQ ID NO: 806) |
| SSSSQQ; | (SEQ ID NO: 807) |
| SSSSQS; | (SEQ ID NO: 808) |
| SSSTAV; | (SEQ ID NO: 809) |
| SSSTNV; | (SEQ ID NO: 810) |
| SSSTSV; | (SEQ ID NO: 811) |
| SSSVAQ; | (SEQ ID NO: 812) |
| SSSVAS; | (SEQ ID NO: 813) |
| SSSVGG; | (SEQ ID NO: 814) |
| SSSVGQ; | (SEQ ID NO: 815) |
| SSSVGS; | (SEQ ID NO: 816) |
| SSSVNG; | (SEQ ID NO: 817) |
| SSSVNS; | (SEQ ID NO: 818) |
| SSSVNT; | (SEQ ID NO: 819) |
| SSSVQG; | (SEQ ID NO: 820) |
| SSSVQN; | (SEQ ID NO: 821) |
| SSSVQS; | (SEQ ID NO: 822) |
| SSSVQT; | (SEQ ID NO: 823) |
| SSSVSG; | (SEQ ID NO: 824) |
| SSSVSN; | (SEQ ID NO: 825) |
| SSSVSQ; | (SEQ ID NO: 826) |
| SSSVSS; | (SEQ ID NO: 827) |
| SSSVST; | (SEQ ID NO: 828) |
| STNGSV; | (SEQ ID NO: 829) |
| STNSGV; | (SEQ ID NO: 830) |
| STNSNV; | (SEQ ID NO: 831) |
| STNTNV; | (SEQ ID NO: 832) |
| STNTQV; | (SEQ ID NO: 833) |
| STNVGS; | (SEQ ID NO: 834) |
| STNVNG; | (SEQ ID NO: 835) |
| STNVNS; | (SEQ ID NO: 836) |
| STNVSG; | (SEQ ID NO: 837) |
| STNVSS; | (SEQ ID NO: 838) |
| STNVTG; | (SEQ ID NO: 839) |
| STNVTS; | (SEQ ID NO: 840) |
| STQQSG; | (SEQ ID NO: 841) |
| STQQSQ; | (SEQ ID NO: 842) |
| STQQSV; | (SEQ ID NO: 843) |
| STQQTQ; | (SEQ ID NO: 844) |
| STQQTS; | (SEQ ID NO: 845) |
| STQQTV; | (SEQ ID NO: 846) |
| STQSNV; | (SEQ ID NO: 847) |
| STQSTV; | (SEQ ID NO: 848) |
| STQTSS; | (SEQ ID NO: 849) |
| STQTSV; | (SEQ ID NO: 850) |
| STQVNS; | (SEQ ID NO: 851) |
| STQVSG; | (SEQ ID NO: 852) |
| STQVSN; | (SEQ ID NO: 853) |
| STQVST; | (SEQ ID NO: 854) |
| STQVTG; | (SEQ ID NO: 855) |
| STQVTN; | (SEQ ID NO: 856) |
| STQVTQ; | (SEQ ID NO: 857) |
| STQVTS; | (SEQ ID NO: 858) |
| STQVTT; | (SEQ ID NO: 859) |
| STSGNV; | (SEQ ID NO: 860) |
| STSNAS; | (SEQ ID NO: 861) |
| STSNGV; | (SEQ ID NO: 862) |
| STSNQV; | (SEQ ID NO: 863) |
| STSNSV; | (SEQ ID NO: 864) |
| STSQAQ; | (SEQ ID NO: 865) |
| STSQGV; | (SEQ ID NO: 866) |
| STSQNQ; | (SEQ ID NO: 867) |
| STSQQQ; | (SEQ ID NO: 868) |
| STSQQV; | (SEQ ID NO: 869) |
| STSQSQ; | (SEQ ID NO: 870) |
| STSSAV; | (SEQ ID NO: 871) |
| STSSGV; | (SEQ ID NO: 872) |
| STSSQQ; | (SEQ ID NO: 873) |
| STSSQS; | (SEQ ID NO: 874) |
| STSSQV; | (SEQ ID NO: 875) |
| STSSSQ; | (SEQ ID NO: 876) |
| STSTAV; | (SEQ ID NO: 877) |
| STSTGV; | (SEQ ID NO: 878) |
| STSTQA; | (SEQ ID NO: 879) |
| STSTQG; | (SEQ ID NO: 880) |
| STSTQT; | (SEQ ID NO: 881) |
| STSTQV; | (SEQ ID NO: 882) |
| STSTSQ; | (SEQ ID NO: 883) |
| STSTSV; | (SEQ ID NO: 884) |
| STSVAG; | (SEQ ID NO: 885) |
| STSVAN; | (SEQ ID NO: 886) |
| STSVAQ; | (SEQ ID NO: 887) |
| STSVAS; | (SEQ ID NO: 888) |
| STSVAT; | (SEQ ID NO: 889) |
| STSVGG; | (SEQ ID NO: 890) |
| STSVGN; | (SEQ ID NO: 891) |
| STSVGQ; | (SEQ ID NO: 892) |
| STSVGS; | (SEQ ID NO: 893) |
| STSVNG; | (SEQ ID NO: 894) |
| STSVNN; | (SEQ ID NO: 895) |
| STSVNS; | (SEQ ID NO: 896) |
| STSVQG; | (SEQ ID NO: 897) |
| STSVQN; | (SEQ ID NO: 898) |
| STSVQQ; | (SEQ ID NO: 899) |
| STSVQS; | (SEQ ID NO: 900) |
| STSVQT; | (SEQ ID NO: 901) |
| STSVSG; | (SEQ ID NO: 902) |
| STSVSN; | (SEQ ID NO: 903) |
| STSVSQ; | (SEQ ID NO: 904) |
| STSVSS; | (SEQ ID NO: 905) |
| STSVST; | (SEQ ID NO: 906) |
| SVNGST; | (SEQ ID NO: 907) |
| SVNGTS; | (SEQ ID NO: 908) |
| SVNQAQ; | (SEQ ID NO: 909) |
| SVNQQQ; | (SEQ ID NO: 910) |
| SVNSGT; | (SEQ ID NO: 911) |
| SVNSNQ; | (SEQ ID NO: 912) |
| SVNSNS; | (SEQ ID NO: 913) |
| SVNSTG; | (SEQ ID NO: 914) |
| SVNSTS; | (SEQ ID NO: 915) |
| SVNTGS; | (SEQ ID NO: 916) |
| SVNTSG; | (SEQ ID NO: 917) |
| SVQQSQ; | (SEQ ID NO: 918) |
| SVQQST; | (SEQ ID NO: 919) |
| SVQQTQ; | (SEQ ID NO: 920) |
| SVQQTS; | (SEQ ID NO: 921) |
| SVQQTT; | (SEQ ID NO: 922) |
| SVQSNQ; | (SEQ ID NO: 923) |
| SVQSNS; | (SEQ ID NO: 924) |
| SVQSSG; | (SEQ ID NO: 925) |
| SVQSSQ; | (SEQ ID NO: 926) |
| SVQSSS; | (SEQ ID NO: 927) |
| SVQSTG; | (SEQ ID NO: 928) |
| SVQSTQ; | (SEQ ID NO: 929) |
| SVQSTS; | (SEQ ID NO: 930) |
| SVQTSG; | (SEQ ID NO: 931) |
| SVQTSN; | (SEQ ID NO: 932) |
| SVQTSS; | (SEQ ID NO: 933) |
| SVQVSN; | (SEQ ID NO: 934) |
| SVSGNT; | (SEQ ID NO: 935) |
| SVSGQS; | (SEQ ID NO: 936) |
| SVSNAQ; | (SEQ ID NO: 937) |
| SVSNAS; | (SEQ ID NO: 938) |
| SVSNGS; | (SEQ ID NO: 939) |
| SVSNGT; | (SEQ ID NO: 940) |
| SVSNQG; | (SEQ ID NO: 941) |
| SVSNQS; | (SEQ ID NO: 942) |
| SVSNQT; | (SEQ ID NO: 943) |
| SVSNST; | (SEQ ID NO: 944) |
| SVSNTG; | (SEQ ID NO: 945) |
| SVSQAQ; | (SEQ ID NO: 946) |
| SVSQAS; | (SEQ ID NO: 947) |
| SVSQGQ; | (SEQ ID NO: 948) |
| SVSQNQ; | (SEQ ID NO: 949) |
| SVSQQG; | (SEQ ID NO: 950) |
| SVSQQQ; | (SEQ ID NO: 951) |
| SVSQQS; | (SEQ ID NO: 952) |
| SVSQSG; | (SEQ ID NO: 953) |
| SVSQSS; | (SEQ ID NO: 954) |
| SVSQTQ; | (SEQ ID NO: 955) |
| SVSSAQ; | (SEQ ID NO: 956) |
| SVSSAS; | (SEQ ID NO: 957) |
| SVSSGG; | (SEQ ID NO: 958) |
| SVSSGS; | (SEQ ID NO: 959) |
| SVSSGT; | (SEQ ID NO: 960) |
| SVSSNG; | (SEQ ID NO: 961) |
| SVSSNQ; | (SEQ ID NO: 962) |
| SVSSNS; | (SEQ ID NO: 963) |
| SVSSNT; | (SEQ ID NO: 964) |
| SVSSQG; | (SEQ ID NO: 965) |
| SVSSQQ; | (SEQ ID NO: 966) |
| SVSSQS; | (SEQ ID NO: 967) |
| SVSSQT; | (SEQ ID NO: 968) |
| SVSSQV; | (SEQ ID NO: 969) |
| SVSSSG; | (SEQ ID NO: 970) |
| SVSSSQ; | (SEQ ID NO: 971) |
| SVSSSS; | (SEQ ID NO: 972) |
| SVSTAG; | (SEQ ID NO: 973) |
| SVSTAS; | (SEQ ID NO: 974) |
| SVSTAT; | (SEQ ID NO: 975) |
| SVSTGS; | (SEQ ID NO: 976) |
| SVSTNS; | (SEQ ID NO: 977) |
| SVSTNT; | (SEQ ID NO: 978) |
| SVSTQQ; | (SEQ ID NO: 979) |
| SVSTQS; | (SEQ ID NO: 980) |
| SVSTQT; | (SEQ ID NO: 981) |
| SVSTSG; | (SEQ ID NO: 982) |
| SVSTSN; | (SEQ ID NO: 983) |
| SVSTSS; | (SEQ ID NO: 984) |
| SVSTST; | (SEQ ID NO: 985) |
| TASQSQ; | (SEQ ID NO: 986) |
| TASQVQ; | (SEQ ID NO: 987) |
| TGSNSV; | (SEQ ID NO: 988) |
| TGSNVS; | (SEQ ID NO: 989) |
| TGSSNV; | (SEQ ID NO: 990) |
| TGSVNS; | (SEQ ID NO: 991) |
| TGSVSN; | (SEQ ID NO: 992) |
| TNSGSV; | (SEQ ID NO: 993) |
| TNSGVS; | (SEQ ID NO: 994) |
| TNSQVQ; | (SEQ ID NO: 995) |
| TNSSGV; | (SEQ ID NO: 996) |
| TNSVGS; | (SEQ ID NO: 997) |
| TNSVSG; | (SEQ ID NO: 998) |
| TQSQSQ; | (SEQ ID NO: 999) |
| TQSQVQ; | (SEQ ID NO: 1000) |
| TQSTVS; | (SEQ ID NO: 1001) |
| TQSVSN; | (SEQ ID NO: 1002) |
| TQSVSS; | (SEQ ID NO: 1003) |
| TSNGVS; | (SEQ ID NO: 1004) |
| TSNVGS; | (SEQ ID NO: 1005) |
| TSNVSG; | (SEQ ID NO: 1006) |
| TSSGNV; | (SEQ ID NO: 1007) |
| TSSGVN; | (SEQ ID NO: 1008) |
| TSSNGV; | (SEQ ID NO: 1009) |
| TSSNSV; | (SEQ ID NO: 1010) |
| TSSNVG; | (SEQ ID NO: 1011) |
| TSSQSQ; | (SEQ ID NO: 1012) |
| TSSSVQ; | (SEQ ID NO: 1013) |
| TSSVGN; | (SEQ ID NO: 1014) |
| TSSVNG; | (SEQ ID NO: 1015) |
| TSSVSG; | (SEQ ID NO: 1016) |
| TSSVSS; | (SEQ ID NO: 1017) |
| TSSVTS; | (SEQ ID NO: 1018) |
| TVNSGS; | (SEQ ID NO: 1019) |
| TVNSSG; | (SEQ ID NO: 1020) |
| TVSGNS; | (SEQ ID NO: 1021) |
| TVSGSN; | (SEQ ID NO: 1022) |
| TVSNGS; | (SEQ ID NO: 1023) |
| TVSNSG; | (SEQ ID NO: 1024) |
| TVSNST; | (SEQ ID NO: 1025) |
| TVSNVG; | (SEQ ID NO: 1026) |
| TVSQNQ; | (SEQ ID NO: 1027) |
| TVSQQV; | (SEQ ID NO: 1028) |
| TVSQSG; | (SEQ ID NO: 1029) |
| TVSQSQ; | (SEQ ID NO: 1030) |
| TVSSGN; | (SEQ ID NO: 1031) |
| TVSSNG; | (SEQ ID NO: 1032) |
| TVSSNQ; | (SEQ ID NO: 1033) |
| TVSSNS; | (SEQ ID NO: 1034) |
| TVSSSQ; | (SEQ ID NO: 1035) |
| TVSTSG; | (SEQ ID NO: 1036) |
| TVSTSN; | (SEQ ID NO: 1037) |
| TVSTSS; | (SEQ ID NO: 1038) |
| TVSTST; | (SEQ ID NO: 1039) |
| VGSTNS; | (SEQ ID NO: 1040) |
| VNSTSG; | (SEQ ID NO: 1041) |
| VNSTSN; | (SEQ ID NO: 1042) |
| VSSQSQ; | (SEQ ID NO: 1043) |
| VSSQVQ; | (SEQ ID NO: 1044) |
| VSSTNG; | (SEQ ID NO: 1045) |
| VTSNSG; | (SEQ ID NO: 1046) |
| and | |
| VTSQSQ. | (SEQ ID NO: 1047) |

Oftentimes, the peptide comprises one or more of the following hexapeptides, where each indicated amino acid is either an L-amino acid or a D-amino acid:

| | |
|---|---|
| SVNLDV | (SEQ ID NO: 5); |
| VEALYL | (SEQ ID NO: 1048); |
| LYQLEN | (SEQ ID NO: 1049); |
| VQIVYK | (SEQ ID NO: 123); |
| GYVIIK | (SEQ ID NO: 1050); |
| SNQNNF | (SEQ ID NO: 1051); |
| SSQVTQ | (SEQ ID NO: 791); |
| SVLTSL | (SEQ ID NO: 1052); |
| SSTNVG | (SEQ ID NO: 1053); |
| SVSSSY | (SEQ ID NO: 1054); |
| GAILSS | (SEQ ID NO: 1055); |
| GAIIGL | (SEQ ID NO: 1056); |
| AIIGLM | (SEQ ID NO: 1057); |
| MVGGVV | (SEQ ID NO: 220); |
| GGVVIA | (SEQ ID NO: 1058). |

Peptides of the present invention are capable of forming fibrils. The corresponding fibrils are typically capable of forming a complex with one or more plasma proteins. Nonlimiting examples of such plasma proteins include (symbol for plasma protein indicated in parenthesis after the plasma protein): Apolipoprotein B-100 (A); Complement C3 (B); Complement C1s (C); Beta-2-glycoprotein 1 (D); Clusterin (E); Coagulation Factor V (F); Complement C1r (G); Apolipoprotein A-I (H); ITIH2 (I); Complement 1qB (J); Apolipoprotein A-IV (K); Complement Factor H (L); Fibrinogen beta chain (M); Complement C4-A (N); Transthyretin (0); SerpinG1 Plasma protease C1 inhibitor (P); Fibrinogen alpha chain (Q); Complement C1qA (R); Vitronectin (S); Serpina-1 Alpha-1-antitrypsin (T); VitaminD-binding protein (U); Haptoglobin-related protein (V); ITIH4 (W); Fibrinogen gamma chain (X); SerpinC1 Antithrombin-III (Y); Apolipoprotein A-2 (Z); Complement Factor H-related protein (AA); Gelsolin (BB); Complement factor B (CC); Alpha-2-HS-glycoprotein (DD); Serum paraoxinase/arylesterase 1 (EE); Complement C5 (FF); Apolipoprotein C5 (GG); Apolipoprotein C-II (HH); Apolipoprotein C-I (II); ITIH1 (JJ); von Willebrand factor (KK); Ceruloplasmin (LL); Apolipoprotein E (MM); Filamin-A (NN); Histidine-rich glycoprotein (00); SerpinF2 alpha-2-antiplasmin (PP); Coagulation factor II Prothrombin (QQ); Coagulation factor X (RR); Vitamin K-dependent protein (SS); Apolipoprotein C-III (TT); Alpha-1-acid glycoprotein 2 (UU); Coagulation factor IX (VV); Apolipoprotein M (WW); Serum Albumin (XX).

Examples of peptide fibril/plasma protein complexes that are capable of being formed include (symbol for hexamer followed by symbol for plasma protein):

1B, 2B, 3B, 4B, 5B, 6B, 7B, 8B, 9B, 10B, 11B, 12B, 13B, 14B, 15B, 16B, 17B, 18B, 19B, 20B, 21B, 22B, 23B, 24B, 25B, 26B, 27B, 28B, 29B, 30B, 31B, 32B, 33B, 34B, 35B, 36B, 37B, 38B, 39B, 40B, 41B, 42B, 43B, 44B, 45B, 46B, 47B, 48B, 49B, 50B, 51B, 52B, 53B, 54B, 55B, 56B, 57B, 58B, 59B, 60B, 61B, 62B, 63B, 64B, 65B, 66B, 67B, 68B, 69B, 70B, 71B, 72B, 73B, 74B, 75B, 76B, 77B, 78B, 79B, 80B, 81B, 82B, 83B, 84B, 85B, 86B, 87B, 88B, 89B, 90B, 91B, 92B, 93B, 94B, 95B, 96B, 97B, 98B, 99B, 100B, 101B, 102B, 103B, 104B, 105B, 106B, 107B, 108B, 109B, 110B, 111B, 112B, 113B, 114B, 115B, 116B, 117B, 118B, 119B, 120B, 121B, 122B, 123B, 124B, 125B, 126B, 127B, 128B, 129B, 130B, 131B, 132B, 133B, 134B, 135B, 136B, 137B, 138B, 139B, 140B, 141B, 142B, 143B, 144B, 145B, 146B, 147B, 148B, 149B, 150B, 151B, 152B, 153B, 154B, 155B, 156B, 157B, 158B, 159B, 160B, 161B, 162B, 163B, 164B, 165B, 166B, 167B, 168B, 169B, 170B, 171B, 172B, 173B, 174B, 175B, 176B, 177B, 178B, 179B, 180B, 181B, 182B, 183B, 184B, 185B, 186B, 187B, 188B, 189B, 190B, 191B, 192B, 193B, 194B, 195B, 196B, 197B, 198B, 199B, 200B, 201B, 202B, 203B, 204B, 205B, 206B, 207B, 208B, 209B, 210B, 211B, 212B, 213B, 214B, 215B, 216B, 217B, 218B, 219B, 220B, 221B, 222B, 223B, 224B, 225B, 226B, 227B, 228B, 229B, 230B, 231B, 232B, 233B, 234B, 235B, 236B, 237B, 238B, 239B, 240B, 241B, 242B, 243B, 244B, 245B, 246B; and 247B-1047B;

1C, 2C, 3C, 4C, 5C, 6C, 7C, 8C, 9C, 10C, 11C, 12C, 13C, 14C, 15C, 16C, 17C, 18C, 19C, 20C, 21C, 22C, 23C, 24C, 25C, 26C, 27C, 28C, 29C, 30C, 31C, 32C, 33C, 34C, 35C, 36C, 37C, 38C, 39C, 40C, 41C, 42C, 43C, 44C, 45C, 46C, 47C, 48C, 49C, 50C, 51C, 52C, 53C, 54C, 55C, 56C, 57C, 58C, 59C, 60C, 61C, 62C, 63C, 64C, 65C, 66C, 67C, 68C, 69C, 70C, 71C, 72C, 73C, 74C, 75C, 76C, 77C, 78C, 79C, 80C, 81C, 82C, 83C, 84C, 85C, 86C, 87C, 88C, 89C, 90C, 91C, 92C, 93C, 94C, 95C, 96C, 97C, 98C, 99C, 100C, 101C, 102C, 103C, 104C, 105C, 106C, 107C, 108C, 109C, 110C, 111C, 112C, 113C, 114C, 115C, 116C, 117C, 118C, 119C, 120C, 121C, 122C, 123C, 124C, 125C, 126C, 127C, 128C, 129C, 130C, 131C, 132C, 133C, 134C, 135C, 136C, 137C, 138C, 139C, 140C, 141C, 142C, 143C, 144C, 145C, 146C, 147C, 148C, 149C, 150C, 151C, 152C, 153C, 154C, 155C, 156C, 157C, 158C, 159C, 160C, 161C, 162C, 163C, 164C, 165C, 166C, 167C, 168C, 169C, 170C, 171C, 172C, 173C, 174C, 175C, 176C, 177C, 178C, 179C, 180C, 181C, 182C, 183C, 184C, 185C, 186C, 187C, 188C, 189C, 190C, 191C, 192C, 193C, 194C, 195C, 196C, 197C, 198C, 199C, 200C, 201C, 202C, 203C, 204C, 205C, 206C, 207C, 208C, 209C, 210C, 211C, 212C, 213C, 214C, 215C, 216C, 217C, 218C, 219C, 220C, 221C, 222C, 223C, 224C, 225C, 226C, 227C, 228C, 229C, 230C, 231C, 232C, 233C, 234C, 235C, 236C, 237C, 238C, 239C, 240C, 241C, 242C, 243C, 244C, 245C, 246C; and 247C-1047C;

1F, 2F, 3F, 4F, 5F, 6F, 7F, 8F, 9F, 10F, 11F, 12F, 13F, 14F, 15F, 16F, 17F, 18F, 19F, 20F, 21F, 22F, 23F, 24F, 25F, 26F, 27F, 28F, 29F, 30F, 31F, 32F, 33F, 34F, 35F, 36F, 37F, 38F, 39F, 40F, 41F, 42F, 43F, 44F, 45F, 46F, 47F, 48F, 49F, 50F, 51F, 52F, 53F, 54F, 55F, 56F, 57F, 58F, 59F, 60F, 61F, 62F, 63F, 64F, 65F, 66F, 67F, 68F, 69F, 70F, 71F, 72F, 73F, 74F, 75F, 76F, 77F, 78F, 79F, 80F, 81F, 82F, 83F, 84F, 85F, 86F, 87F, 88F, 89F, 90F, 91F, 92F, 93F, 94F, 95F, 96F, 97F, 98F, 99F, 100F, 101F, 102F, 103F, 104F, 105F, 106F, 107F, 108F, 109F, 110F, 111F, 112F, 113F, 114F, 115F, 116F, 117F, 118F, 119F, 120F, 121F, 122F, 123F, 124F, 125F, 126F, 127F, 128F, 129F, 130F, 131F, 132F, 133F, 134F, 135F, 136F, 137F, 138F, 139F, 140F, 141F, 142F, 143F, 144F, 145F, 146F, 147F, 148F, 149F, 150F, 151F, 152F, 153F, 154F, 155F, 156F, 157F, 158F, 159F, 160F, 161F, 162F, 163F, 164F, 165F, 166F, 167F, 168F, 169F, 170F, 171F, 172F, 173F, 174F, 175F, 176F, 177F, 178F, 179F, 180F, 181F, 182F, 183F, 184F, 185F, 186F, 187F, 188F, 189F, 190F, 191F, 192F, 193F, 194F, 195F, 196F, 197F, 198F, 199F, 200F, 201F, 202F, 203F, 204F, 205F, 206F, 207F, 208F, 209F, 210F, 211F, 212F, 213F, 214F, 215F, 216F, 217F, 218F, 219F, 220F, 221F, 222F, 223F, 224F, 225F, 226F, 227F, 228F, 229F, 230F, 231F, 232F, 233F, 234F, 235F, 236F, 237F, 238F, 239F, 240F, 241F, 242F, 243F, 244F, 245F, 246F; and 247F-1047F;

1G, 2G, 3G, 4G, 5G, 6G, 7G, 8G, 9G, 10G, 11G, 12G, 13G, 14G, 15G, 16G, 17G, 18G, 19G, 20G, 21G, 22G, 23G, 24G, 25G, 26G, 27G, 28G, 29G, 30G, 31G, 32G, 33G, 34G, 35G, 36G, 37G, 38G, 39G, 40G, 41G, 42G, 43G, 44G, 45G, 46G, 47G, 48G, 49G, 50G, 51G, 52G, 53G, 54G, 55G, 56G, 57G, 58G, 59G, 60G, 61G, 62G, 63G, 64G, 65G, 66G, 67G, 68G, 69G, 70G, 71G, 72G, 73G, 74G, 75G, 76G, 77G, 78G, 79G, 80G, 81G, 82G, 83G, 84G, 85G, 86G, 87G, 88G, 89G, 90G, 91G, 92G, 93G, 94G, 95G, 96G, 97G, 98G, 99G, 100G, 101G, 102G, 103G, 104G, 105G, 106G, 107G, 108G, 1090, 110G, 1110, 112G, 113G, 114G, 115G, 116G, 117G, 118G, 119G, 120G, 121G, 122G, 123G, 124G, 125G, 126G, 127G, 128G, 129G, 130G, 131G, 1320, 133G, 134G, 135G, 136G, 137G, 138G, 139G, 140G, 141G, 142G, 143G, 144G, 145G, 146G, 147G, 148G, 149G, 150G, 151G, 152G, 153G, 154G, 155G, 156G, 157G, 158G, 159G, 160G, 161G, 162G, 163G, 164G, 165G, 166G, 167G, 168G, 169G, 170G, 171G, 172G, 173G, 1740, 175G, 176G, 177G, 178G, 179G, 180G, 181G, 182G, 183G, 184G, 185G, 186G, 187G, 188G, 189G, 190G, 191G, 192G, 193G, 194G, 195G, 196G, 197G, 198G, 199G, 200G, 201G, 202G, 203G, 204G, 205G, 206G, 207G, 208G, 209G, 210G, 211G, 212G, 213G, 214G, 215G, 216G, 217G, 218G, 219G, 220G, 221G, 222G, 223G, 224G, 225G, 226G, 227G, 228G, 229G, 230G, 231G, 232G, 233G, 234G, 235G, 236G, 237G, 238G, 239G, 240G, 241G, 242G, 243G, 244G, 245G, 246G; and 247G-1047G;

1M, 2M, 3M, 4M, 5M, 6M, 7M, 8M, 9M, 10M, 11M, 12M, 13M, 14M, 15M, 16M, 17M, 18M, 19M, 20M, 21M, 22M, 23M, 24M, 25M, 26M, 27M, 28M, 29M, 30M, 31M, 32M, 33M, 34M, 35M, 36M, 37M, 38M, 39M, 40M, 41M, 42M, 43M, 44M, 45M, 46M, 47M, 48M, 49M, 50M, 51M, 52M, 53M, 54M, 55M, 56M, 57M, 58M, 59M, 60M, 61M, 62M, 63M, 64M, 65M, 66M, 67M, 68M, 69M, 70M, 71M, 72M, 73M, 74M, 75M, 76M, 77M, 78M, 79M, 80M, 81M, 82M, 83M, 84M, 85M, 86M, 87M, 88M, 89M, 90M, 91M, 92M, 93M, 94M, 95M, 96M, 97M, 98M, 99M, 100M, 101M, 102M, 103M, 104M, 105M, 106M, 107M, 108M, 109M, 110M, 111M, 112M, 113M, 114M, 115M, 116M, 117M, 118M, 119M, 120M, 121M, 122M, 123M, 124M, 125M, 126M, 127M, 128M, 129M, 130M, 131M, 132M, 133M, 134M, 135M, 136M, 137M, 138M, 139M, 140M, 141M, 142M, 143M, 144M, 145M, 146M, 147M, 148M, 149M, 150M, 151M, 152M, 153M, 154M, 155M, 156M, 157M, 158M, 159M, 160M, 161M, 162M, 163M, 164M, 165M, 166M, 167M, 168M, 169M, 170M, 171M, 172M, 173M, 174M, 175M, 176M, 177M, 178M, 179M, 180M, 181M, 182M, 183M, 184M, 185M, 186M, 187M, 188M, 189M, 190M, 191M, 192M, 193M, 194M, 195M, 196M, 197M, 198M, 199M, 200M, 201M, 202M, 203M, 204M, 205M, 206M, 207M, 208M, 209M, 210M, 211M, 212M, 213M, 214M, 215M, 216M, 217M, 218M, 219M, 220M, 221M, 222M, 223M, 224M, 225M, 226M, 227M, 228M, 229M, 230M, 231M, 232M, 233M, 234M, 235M, 236M, 237M, 238M, 239M, 240M, 241M, 242M, 243M, 244M, 245M, 246M; and 247M-1047M;

1J, 2J, 3J, 4J, 5J, 6J, 7.1, 8J, 9J, 10J, 11J, 12J, 13J, 14J, 15J, 16J, 17J, 18J, 19J, 20J, 21J, 22J, 23J, 24.1, 25J, 26J, 27J, 28J, 29J, 30J, 31J, 32J, 33J, 34J, 35J, 36J, 37J, 38J, 39J, 40J, 41J, 42J, 43J, 44J, 45J, 46J, 47J, 48.1, 49J, 50J, 51J, 52J, 53J, 54J, 55J, 56J, 57J, 58J, 59J, 60J, 61J, 62J, 63J, 64J, 65J, 66J, 67J, 68J, 69J, 70J, 71.1, 72J, 73.1, 74J, 75J, 76J, 77J, 78J, 79J, 80J, 81J, 82J, 83J, 84J, 85J, 86J, 87J, 88J, 89J, 90J, 91J, 92J, 93J, 94J, 95J, 96J, 97J, 98J, 99J, 100J, 101J, 102J, 103J, 104J, 105J, 106J, 107J, 108J, 109J, 110J, 111J, 112J, 113J, 114J, 115J, 116J, 117J, 118J, 119J, 120J, 121J, 122J, 123J, 124.1, 125J, 126J, 127J, 128J, 129J, 130J, 131J, 132J, 133J, 134J, 135J, 136J, 137J, 138J, 139J, 140J, 141J, 142J, 143J, 144J, 145.1, 146J, 147J, 148J, 149J, 150J, 151J, 152J, 153J, 154J, 155J, 156J, 157J, 158J, 159J, 160J, 161J, 162J, 163J, 164J, 165J, 166J, 167J, 168J, 169J, 170J, 171J, 172J, 173J, 174J, 175J, 176J, 177J, 178J, 179J, 180J, 181J, 182J, 183.1, 184J, 185J, 186.1, 187J, 188J, 189.1, 190J, 191J, 192J, 193J, 194J, 195J, 196J, 197J, 198J, 199J, 200J, 201J, 202J, 203J, 204J, 205J, 206J, 207J, 208J, 209J, 210J, 211J, 212J, 213J, 214J, 215J, 216J, 217J, 218J, 219J, 220J, 221J, 222J, 223J, 224J, 225J, 226J, 227J, 228J, 229J, 230J, 231J, 232J, 233J, 234.1, 235J, 236J, 237J, 238J, 239J, 240J, 241J, 242J, 243J, 244J, 245J, 246J; and 247J-1047J;

1L, 2L, 3L, 4L, 5L, 6L, 7L, 8L, 9L, 10L, 11L, 12L, 13L, 14L, 15L, 16L, 17L, 18L, 19L, 20L, 21L, 22L, 23L, 24L, 25L, 26L, 27L, 28L, 29L, 30L, 31L, 32L, 33L, 34L, 35L, 36L, 37L, 38L, 39L, 40L, 41L, 42L, 43L, 44L, 45L, 46L, 47L, 48L, 49L, 50L, 51L, 52L, 53L, 54L, 55L, 56L, 57L, 58L, 59L, 60L, 61L, 62L, 63L, 64L, 65L, 66L, 67L, 68L, 69L, 70L, 71L, 72L, 73L, 74L, 75L, 76L, 77L, 78L, 79L, 80L, 81L, 82L, 83L, 84L, 85L, 86L, 87L, 88L, 89L, 90L, 91L, 92L, 93L, 94L, 95L, 96L, 97L, 98L, 99L, 100L, 101L, 102L, 103L, 104L, 105L, 106L, 107L, 108L, 109L, 110L, 111L, 112L, 113L, 114L, 115L, 116L, 117L, 118L, 119L, 120L, 121L, 122L, 123L, 124L, 125L, 126L, 127L, 128L, 129L, 130L, 131L, 132L, 133L, 134L, 135L, 136L, 137L, 138L, 139L, 140L, 141L, 142L, 143L, 144L, 145L, 146L, 147L, 148L, 149L, 150L, 151L, 152L, 153L, 154L, 155L, 156L, 157L, 158L, 159L, 160L, 161L, 162L, 163L, 164L, 165L, 166L, 167L, 168L, 169L, 170L, 171L, 172L, 173L, 174L, 175L, 176L, 177L, 178L, 179L, 180L, 181L, 182L, 183L, 184L, 185L, 186L, 187L, 188L, 189L, 190L, 191L, 192L, 193L, 194L, 195L, 196L, 197L, 198L, 199L, 200L, 201L, 202L, 203L, 204L, 205L, 206L, 207L, 208L, 209L, 210L, 211L, 212L, 213L, 214L, 215L, 216L, 217L, 218L, 219L, 220L, 221L, 222L, 223L, 224L, 225L, 226L, 227L, 228L, 229L, 230L, 231L, 232L, 233L, 234L, 235L, 236L, 237L, 238L, 239L, 240L, 241L, 242L, 243L, 244L, 245L, 246L; and 247L-1047L;

1V, 2V, 3V, 4V, 5V, 6V, 7V, 8V, 9V, 10V, 11V, 12V, 13V, 14V, 15V, 16V, 17V, 18V, 19V, 20V, 21V, 22V, 23V, 24V, 25V, 26V, 27V, 28V, 29V, 30V, 31V, 32V, 33V, 34V, 35V, 36V, 37V, 38V, 39V, 40V, 41V, 42V, 43V, 44V, 45V, 46V, 47V, 48V, 49V, 50V, 51V, 52V, 53V, 54V, 55V, 56V, 57V, 58V, 59V, 60V, 61V, 62V, 63V, 64V, 65V, 66V, 67V, 68V, 69V, 70V, 71V, 72V, 73V, 74V, 75V, 76V, 77V, 78V, 79V, 80V, 81V, 82V, 83V, 84V, 85V, 86V, 87V, 88V, 89V, 90V, 91V, 92V, 93V, 94V, 95V, 96V, 97V, 98V, 99V, 100V, 101V, 102V, 103V, 104V, 105V, 106V, 107V, 108V, 109V, 110V, 111V, 112V, 113V, 114V, 115V, 116V, 117V, 118V, 119V, 120V, 121V, 122V, 123V, 124V, 125V, 126V, 127V, 128V, 129V, 130V, 131V, 132V, 133V, 134V, 135V, 136V, 137V, 138V, 139V, 140V, 141V, 142V, 143V, 144V, 145V, 146V, 147V, 148V, 149V, 150V, 151V, 152V, 153V, 154V, 155V, 156V, 157V, 158V, 159V, 160V, 161V, 162V, 163V, 164V, 165V, 166V, 167V, 168V, 169V, 170V, 171V, 172V, 173V, 174V, 175V, 176V, 177V, 178V, 179V, 180V, 181V, 182V, 183V, 184V, 185V, 186V, 187V, 188V, 189V, 190V, 191V, 192V, 193V, 194V, 195V, 196V, 197V, 198V, 199V, 200V, 201V, 202V, 203V, 204V, 205V, 206V, 207V, 208V, 209V, 210V, 211V, 212V, 213V, 214V, 215V, 216V, 217V, 218V, 219V, 220V, 221V, 222V, 223V, 224V, 225V, 226V, 227V, 228V, 229V, 230V, 231V, 232V, 233V, 234V, 235V, 236V, 237V, 238V, 239V, 240V, 241V, 242V, 243V, 244V, 245V, 246V; and 247V-1047V;

1KK, 2KK, 3KK, 4KK, 5KK, 6KK, 7KK, 8KK, 9KK, 10KK, 11KK, 12KK, 13KK, 14KK, 15KK, 16KK, 17KK, 18KK, 19KK, 20KK, 21KK, 22KK, 23KK, 24KK, 25KK, 26KK, 27KK, 28KK, 29KK, 30KK, 31KK, 32KK, 33KK, 34KK, 35KK, 36KK, 37KK, 38KK, 39KK, 40KK, 41KK, 42KK, 43KK, 44KK, 45KK, 46KK, 47KK, 48KK, 49KK, 50KK, 51KK, 52KK, 53KK, 54KK, 55KK, 56KK, 57KK, 58KK, 59KK, 60KK, 61KK, 62KK, 63KK, 64KK, 65KK, 66KK, 67KK, 68KK, 69KK, 70KK, 71KK, 72KK, 73KK, 74KK, 75KK, 76KK, 77KK, 78KK, 79KK, 80KK, 81KK, 82KK, 83KK, 84KK, 85KK, 86KK, 87KK, 88KK, 89KK, 90KK, 91KK, 92KK, 93KK, 94KK, 95KK, 96KK, 97KK, 98KK, 99KK, 100KK, 101KK, 102KK, 103KK, 104KK, 105KK, 106KK, 107KK, 108KK, 109KK, 110KK, 111KK, 112KK, 113KK, 114KK, 115KK, 116KK, 117KK, 118KK, 119KK, 120KK, 121KK, 122KK, 123KK, 124KK, 125KK, 126KK, 127KK, 128KK, 129KK, 130KK, 131KK, 132KK, 133KK, 134KK, 135KK, 136KK, 137KK, 138KK, 139KK, 140KK, 141KK, 142KK, 143KK, 144KK, 145KK, 146KK, 147KK, 148KK, 149KK, 150KK, 151KK, 152KK, 153KK, 154KK, 155KK, 156KK, 157KK, 158KK, 159KK, 160KK, 161KK, 162KK, 163KK, 164KK, 165KK, 166KK, 167KK, 168KK, 169KK, 170KK, 171KK, 172KK, 173KK, 174KK, 175KK, 176KK, 177KK, 178KK, 179KK, 180KK, 181KK, 182KK, 183KK, 184KK, 185KK, 186KK, 187KK, 188KK, 189KK, 190KK, 191KK, 192KK, 193KK, 194KK, 195KK, 196KK, 197KK, 198KK, 199KK, 200KK, 201KK, 202KK, 203KK, 204KK, 205KK, 206KK, 207KK, 208KK, 209KK, 210KK, 211KK, 212KK, 213KK, 214KK, 215KK, 216KK, 217KK, 218KK, 219KK, 220KK, 221KK, 222KK, 223KK, 224KK, 225KK, 226KK, 227KK, 228KK, 229KK, 230KK, 231KK, 232KK, 233KK, 234KK, 235KK, 236KK, 237KK, 238KK, 239KK, 240KK, 241KK, 242KK, 243KK, 244KK, 245KK, 246KK; and 247KK-1047KK;

1LL, 2LL, 3LL, 4LL, 5LL, 6LL, 7LL, 8LL, 9LL, 10LL, 11LL, 12LL, 13LL, 14LL, 15LL, 16LL, 17LL, 18LL, 19LL, 20LL, 21LL, 22LL, 23LL, 24LL, 25LL, 26LL, 27LL, 28LL, 29LL, 30LL, 31LL, 32LL, 33LL, 34LL, 35LL, 36LL, 37LL, 38LL, 39LL, 40LL, 41LL, 42LL, 43LL, 44LL, 45LL, 46LL, 47LL, 48LL, 49LL, 50LL, 51LL, 52LL, 53LL, 54LL, 55LL, 56LL, 57LL, 58LL, 59LL, 60LL, 61LL, 62LL, 63LL, 64LL, 65LL, 66LL, 67LL, 68LL, 69LL, TOLL, 71LL, 72LL, 73LL, 74LL, 75LL, 76LL, 77LL, 78LL, 79LL, 80LL, 81LL, 82LL, 83LL, 84LL, 85LL, 86LL, 87LL, 88LL, 89LL, 90LL, 91LL, 92LL, 93LL, 94LL, 95LL, 96LL, 97LL, 98LL, 99LL, 100LL, 101LL, 102LL, 103LL, 104LL, 105LL, 106LL, 107LL, 108LL, 109LL, 110LL, 111LL, 112LL, 113LL, 114LL, 115LL, 116LL, 117LL, 118LL, 119LL, 120LL, 121LL, 122LL, 123LL, 124LL, 125LL, 126LL, 127LL, 128LL, 129LL, 130LL, 131LL, 132LL, 133LL, 134LL, 135LL, 136LL, 137LL, 138LL, 139LL, 140LL, 141LL, 142LL, 143LL, 144LL, 145LL, 146LL, 147LL, 148LL, 149LL, 150LL, 151LL, 152LL, 153LL, 154LL, 155LL, 156LL, 157LL, 158LL, 159LL, 160LL, 161LL, 162LL, 163LL, 164LL, 165LL, 166LL, 167LL, 168LL, 169LL, 170LL, 171LL, 172LL, 173LL, 174LL, 175LL, 176LL, 177LL, 178LL, 179LL, 180LL, 181LL, 182LL, 183LL, 184LL, 185LL, 186LL, 187LL, 188LL, 189LL, 190LL, 191LL, 192LL, 193LL, 194LL, 195LL, 196LL, 197LL, 198LL, 199LL, 200LL, 201LL, 202LL, 203LL, 204LL, 205LL, 206LL, 207LL, 208LL, 209LL, 210LL, 211LL, 212LL, 213LL, 214LL, 215LL, 216LL, 217LL, 218LL, 219LL, 220LL, 221LL, 222LL, 223LL, 224LL, 225LL, 226LL, 227LL, 228LL, 229LL, 230LL, 231LL, 232LL, 233LL, 234LL, 235LL, 236LL, 237LL, 238LL, 239LL, 240LL, 241LL, 242LL, 243LL, 244LL, 245LL, 246LL; and 247LL-1047LL;

1XX, 2XX, 3XX, 4XX, 5XX, 6XX, 7XX, 8XX, 9XX, 10XX, 11XX, 12XX, 13XX, 14XX, 15XX, 16XX, 17XX, 18XX, 19XX, 20XX, 21XX, 22XX, 23XX, 24XX, 25XX, 26XX, 27XX, 28XX, 29XX, 30XX, 31XX, 32XX, 33XX, 34XX, 35XX, 36XX, 37XX, 38XX, 39XX, 40XX, 41XX, 42XX, 43XX, 44XX, 45XX, 46XX, 47XX, 48XX, 49XX, 50XX, 51XX, 52XX, 53XX, 54XX, 55XX, 56XX, 57XX, 58XX, 59XX, 60XX, 61XX, 62XX, 63XX, 64XX, 65XX, 66XX, 67XX, 68XX, 69XX, 70XX, 71XX, 72XX, 73XX, 74XX, 75XX, 76XX, 77XX, 78XX, 79XX, 80XX, 81XX, 82XX, 83XX, 84XX, 85XX, 86XX, 87XX, 88XX, 89XX, 90XX, 91XX, 92XX, 93XX, 94XX, 95XX, 96XX, 97XX, 98XX, 99XX, 100XX, 101XX, 102XX, 103XX, 104XX, 105XX, 106XX, 107XX, 108XX, 109XX, 110XX, 111XX, 112XX, 113XX, 114XX, 115XX, 116XX, 117XX, 118XX, 119XX, 120XX, 121XX, 122XX, 123XX, 124XX, 125XX, 126XX, 127XX, 128XX, 129XX, 130XX, 131XX, 132XX, 133XX, 134XX, 135XX, 136XX, 137XX, 138XX, 139XX, 140XX, 141XX, 142XX, 143XX, 144XX, 145XX, 146XX, 147XX, 148XX, 149XX, 150XX, 151XX, 152XX, 153XX, 154XX, 155XX, 156XX, 157XX, 158XX, 159XX, 160XX, 161XX, 162XX, 163XX, 164XX, 165XX, 166XX, 167XX, 168XX, 169XX, 170XX, 171XX, 172XX, 173XX, 174XX, 175XX, 176XX, 177XX, 178XX, 179XX, 180XX, 181XX, 182XX, 183XX, 184XX, 185XX, 186XX, 187XX, 188XX, 189XX, 190XX, 191XX, 192XX, 193XX, 194XX, 195XX, 196XX, 197XX, 198XX, 199XX, 200XX, 201XX, 202XX, 203XX, 204XX, 205XX, 206XX, 207XX, 208XX, 209XX, 210XX, 211XX, 212XX, 213XX, 214XX, 215XX, 216XX, 217XX, 218XX, 219XX, 220XX, 221XX, 222XX, 223XX, 224XX, 225XX, 226XX, 227XX, 228XX, 229XX, 230XX, 231XX, 232XX, 233XX, 234XX, 235XX, 236XX, 237XX, 238XX, 239XX, 240XX, 241XX, 242XX, 243XX, 244XX, 245XX, 246XX; and 247XX-1047XX;

In another composition aspect, the present invention is directed to fibrils comprising four or more of peptides comprising one or more of hexapeptides that generate a free energy of formation of −23 kcal/mol or less (i.e., more negative) when the hexapeptide sequence in subjected to the Rosetta-Profile algorithm.

In yet another compositions aspect, the present invention is directed to complexes comprising one or more fibrils of the present invention and one or more plasma proteins, including plasma proteins A-WW listed above.

Where the peptides of the present invention are formulated to be administered to a mammal, the formulation is typically chosen to provide a homogeneous solution comprising peptides. Peptides with amino acid subunits that are anionic at pH 7.4 are oftentimes formulated in water at a pH of 3-5. Peptides with amino acid subunits that are cationic at pH 7.4 are oftentimes formulated in water at pH 7.4 or a pH above 9 (e.g., 10). In certain cases, a solubility enhancer may be included in the formulation. Nonlimiting examples include DMSO and ethanol.

Compositions of the present invention are used to treat mammalian diseases. Compositions of the present invention may be used to treat inflammatory conditions. Nonlimiting examples of such inflammatory conditions include: multiple sclerosis; stroke; cardiac ischemia-reperfusion injury; retinal ischemia-reperfusion injury; macular degeneration; glaucoma; retinitis pigmentosa; rheumatoid arthritis; Type 1 diabetes; Type 2 diabetes; amyloid neuropathy; and amyloid nephropathy.

In a method aspect of the present invention, a method of treating an inflammatory condition is provided. The method comprises administration of a therapeutically effective amount of a composition of the present invention, in a suitable formulation, to a mammal experiencing an inflammatory condition.

Administration of the formulation may be performed by any method producing the desired therapeutic results. Nonlimiting examples of administration methods include: oral; sublingual; buccal; intranasal; intrapulmonary; intraceregral; intravenous; intramuscular; rectal; and, epidural.

### Examples

### Methods

EAE was induced by procedures previously described. See, Steinman, L. & Zamvil, S. S. How to successfully apply animal studies in experimental allergic encephalomyelitis to research on multiple sclerosis. Ann Neurol 60, 12-21, doi:10.1002/ana.20913 (2006). When animals exhibited hindlimb weakness they were injected in the peritoneum with either 1 μg of peptide or PBS daily. Sera from EAE mice were collected following two days of treatment with 10 μg HspB1 protein, 1 μg tau 623-628 (VQIVYK)(SEQ ID NO: 123), or PBS. Twenty-six cytokines were analyzed by multiplex-bead-analysis.

The capacity of the proteins and peptides to inhibit DTT induced aggregation of the beta chain of insulin was assayed using procedures described previously by several authors. See, for example, Hong, D. P. & Fink, A. L. Independent heterologous fibrillation of insulin and its B-chain peptide. Biochemistry 44, 16701-16709, doi:10.1021/bi051658y (2005). Also see, Hong, D. P., Ahmad, A. & Fink, A. L. Fibrillation of human insulin A and B chains. Biochemistry 45, 9342-9353, doi:10.1021/bi0604936 (2006). The aggregation was measured by the increase in absorption at 360 nm as a function of time over twenty minutes.

The relative amount of amyloid present in each solution was measured by combining solutions of the hexamers (200 mg 50 ml) dissolved in 100 mM MES pH 7.4 and 50 ml of a 1M buffer solution at the appropriate pH (glycine, pH 3; acetate, pH 4; citrate, pH 5; MES, pH 6; Tris, pH 7; Tris, pH 8; glycine, pH 9; bicarbonate, pH 10) and incubated at 37° C. ThT (10 ml of 10 mM solution) was added and the resultant emission fluorescence at 485 nm for each sample after excitation at 440 nm was measured using a SpectraMax 190 fluorescent microtiter plate reader.

The ligands for the amyloid fibrils were identified by incubating 50 mgs of biotinylated fibrils of Tau 623-628 with fresh human plasma at 37° C. for one hour after which 50 ml of streptavidin sepharose beads were added and the mixture was incubated an additional hour at 37° C. The resin was separated from the plasma, washed, and the ligands were eluted. The resin was separated from the eluted proteins as previously described. See, Rothbard, J. B. et al. Therapeutic effects of systemic administration of chaperone alphaB-crystallin associated with binding proinflammatory plasma proteins. J Biol Chem 287, 9708-9721, doi:M111.337691 [pii] 10.1074/jbc.M111.337691 (2012). The ligands were precipitated by the addition of TCA, and resultant precipitates were reconstituted, reduced and alkylated, trypsinized, and the resultant peptides were separated by HPLC. Liquid chromatography and mass spectral analysis were done as previously described. Id.

Experiments

1. Therapeutic Effectiveness of Amyloidogenic Hexamers.

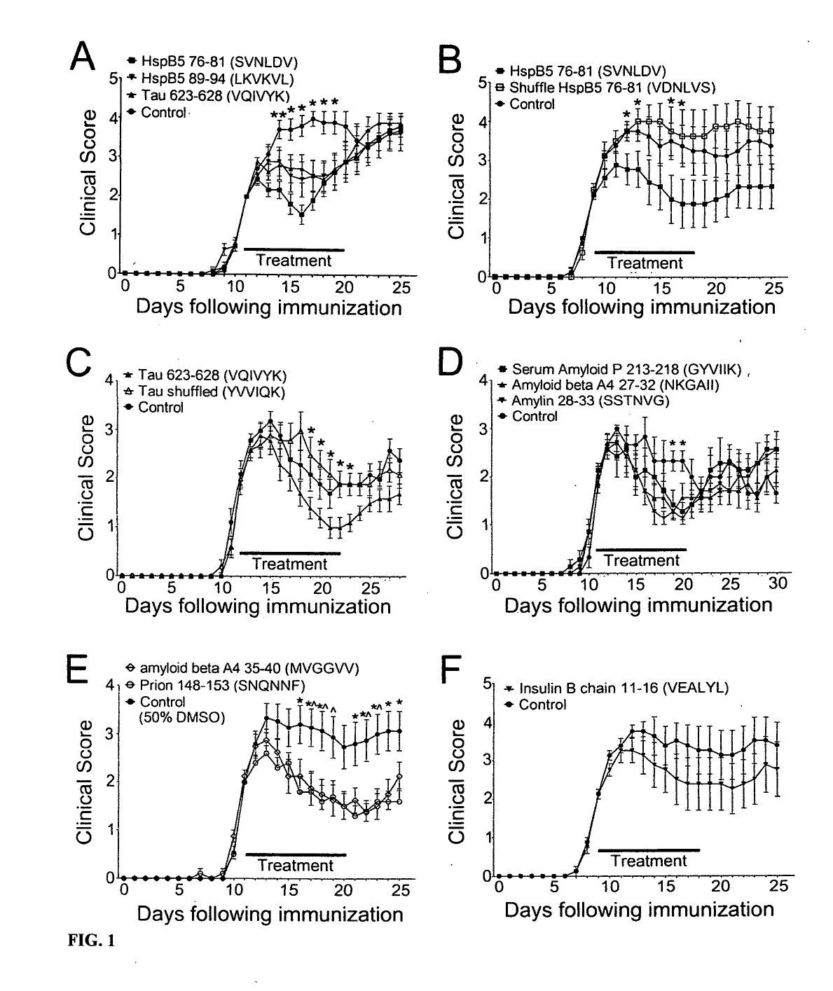

Groups of ten mice were injected daily with 1 μg of the peptides listed in Table 1 (shown below) beginning at onset of hindlimb weakness. PBS or PBS containing 50% DMSO was injected in control littermates. Hexamers corresponding to residues 76-81, 89-94, and 623-628 of Tau effectively reduced the paralytic symptoms of EAE (Panel A of FIG. 1). Bars represent the duration of the treatment. Values in the graph represent the mean+/−S.E.M. *p<0.05 for SVNDLV (SEQ ID NO: 1059), LKVKVL (SEQ ID NO: 1060), VQIVYK (SEQ ID NO: 123) by the Mann Whitney U test. The shuffled analogs of HspB5 76-81 and Tau 623-628 were ineffective (Panels B&C) +p<0.05 for VQIVYK (SEQ ID NO: 123) by Mann Whitney U test. Hexamers corresponding to serum amyloid P 213-218, A beta A4 27-32, amylin 28-33 were therapeutic (Panel D of FIG. 1) *p<0.05 for GYVIIK (SEQ ID NO: 1050), NKGAII (SEQ ID NO: 1061), and SSTNVG (SEQ ID NO: 1053) by the Mann Whitney U test. Two poorly soluble peptides, prion 148-153 and A beta A4 35-40, were therapeutic when administered in 50% DMSO (Panel E) *p<0.05 for SNQNNF (SEQ ID NO: 1051) and ̂p<0.05 for MVGGVV (SEQ ID NO: 220) by the Mann Whitney U test. Administration of insulin B chain 11-16 did not statistically significantly reduce the symptoms of EAE (Panel F of FIG. 1).

TABLE 1
Hexameric peptides used in the study segregated by composition and/or
orientation in the fibril. Those whose crystal structure has been published
are in italics with their denoted classes, with the hydrophobic amino acids
highlighted in dark grey, acidic residues and
basic amino acids in light grey.

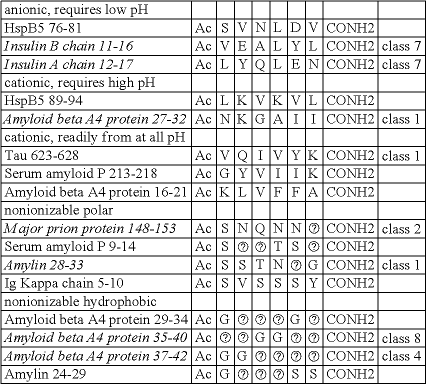

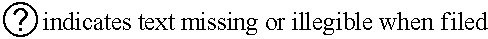

2. Modulation of Plasma Cytokine Levels.

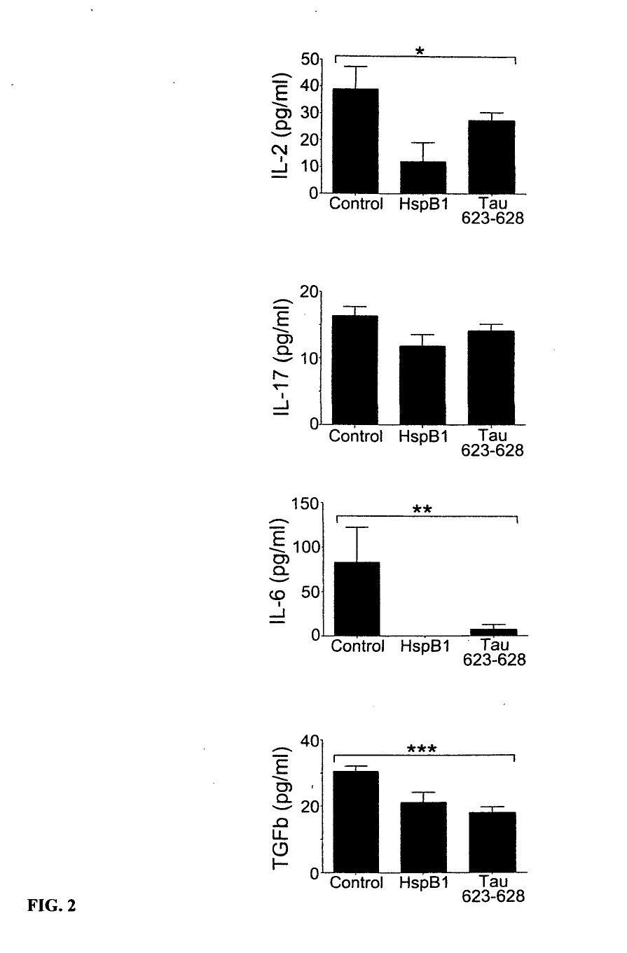

To determine whether a hexamer modulated the plasma cytokine levels equivalent to an intact small heat shock protein, groups of five mice were treated after the onset of hindlimb weakness with daily injections of either 1 μg of Tau 623-628 or 10 μg of HspB1 for two days and bled 12 hours later. Assaying the levels of 26 cytokines in the serum of untreated and treated mice revealed that administration of either HspB 1 or the tau hexamer resulted in a dramatic reduction of IL-6, with smaller reductions in IL-2, TGF-β, and IL-17. See FIG. 2.

3. pH Dependent Amyloid Formation.

The ability of each hexamer listed in Table 1 to form amyloid fibrils in aqueous buffers was examined. Each of the hexamers (200 mg 50 ml) was added to 100 ml of 100 mM MES pH 7.4 and combined with 50 ml of a 1M buffer solution at the appropriate pH (glycine, pH 3; acetate, pH 4; citrate, pH 5; MES, pH 6; Tris, pH 7; Tris, pH 8; glycine, pH 9; bicarbonate, pH 10) and incubated at 37° C. ThT (10 ml of 10 mM solution) was added and the resultant fluorescence at 485 nm from excitation at 440 nm was measured using a fluorescent plate reader. The peptides can be segregated into sets A-E: an anionic set forming fibrils at pH 3, pH 4, and pH 5 (A), a cationic group forming fibrils only at pH 10 (B), three cationic sequences that form amyloid fibrils at all pHs measured (C), a nonionizable set of sequences weakly binding ThT at all pHs (D), and a hydrophobic set of sequences that bind ThT at all pHs tested (E). Only a fraction of the peptides formed amyloid fibrils when dissolved in PBS pH 7.4, as measured by staining with thioflavin T (ThT). Their differential propensity to form amyloid fibrils was used to help segregate them into functional sets (Table 1).

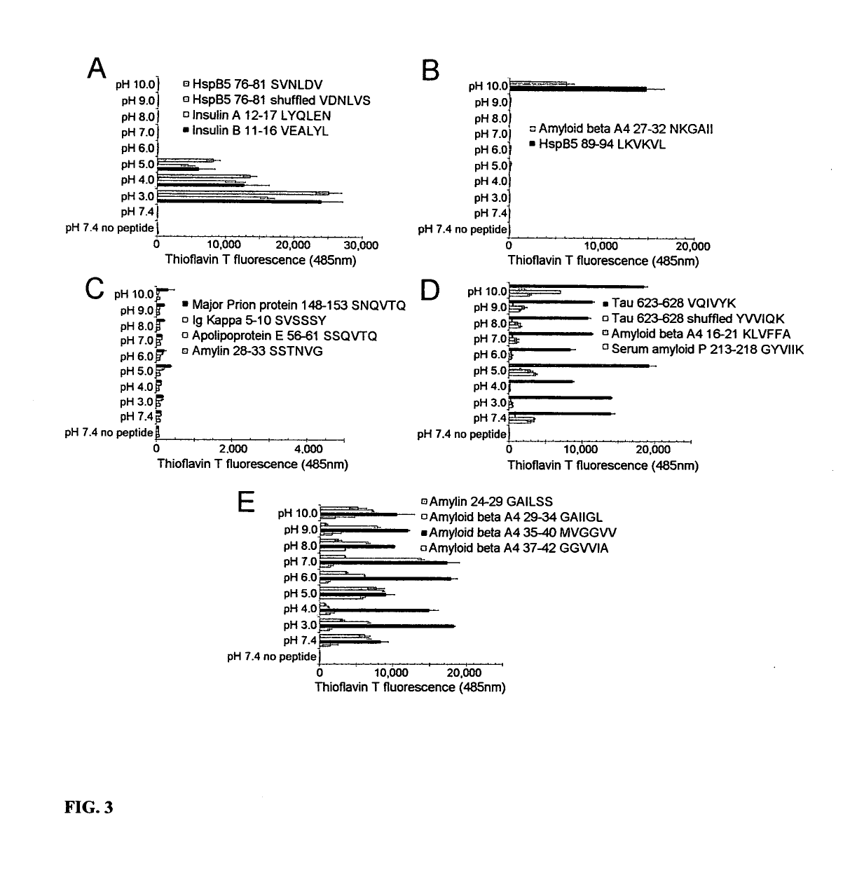

The three peptides containing acidic residues, HspB5 76-81, insulin B chain 11-16, and insulin A chain 12-17, effectively formed amyloid fibrils only in a range of pH 3 to pH 5 when the acidic residue would be protonated (Panel A of FIG. 3). Reciprocally, a second set of two peptides containing lysine, HspB5 89-94 and amyloid b A4 27-32, bound ThT only at pH 10 when the lysine would not be protonated (Panel B of FIG. 3). In contrast, three peptides containing lysine, Tau 623-628, amyloid beta A4 16-21, and serum amyloid P 213-218, formed amyloid fibrils in buffers from pH 3-10, (Panel D of FIG. 3). Two other sets were identified, both of which contained nonionizable amino acids.

4. Correlation of ThT Staining with Molecular Chaperone Function.

To determine whether the ThT staining observed in Experiment 4 (above) correlated with molecular chaperone function, each of the peptides was assayed for its ability to inhibit insulin aggregation. The effect of ThT on insulin aggregation was measured as a control. Neither the addition of 10 mM ThT nor ThT bound to Tau 623-628 affected the rate of insulin aggregation in the former case or inhibition by the Tau peptide in the latter case. Insulin aggregation is pH dependent, only allowing a dynamic range for the assay at pH between 5 and 8, which allows only a fraction of the pH range used in the ThT staining experiments to be analyzed. See, Nielsen, L. et al. Effect of environmental factors on the kinetics of insulin fibril formation: elucidation of the molecular mechanism. Biochemistry 40, 6036-6046, doi:bi002555c [pii] (2001).

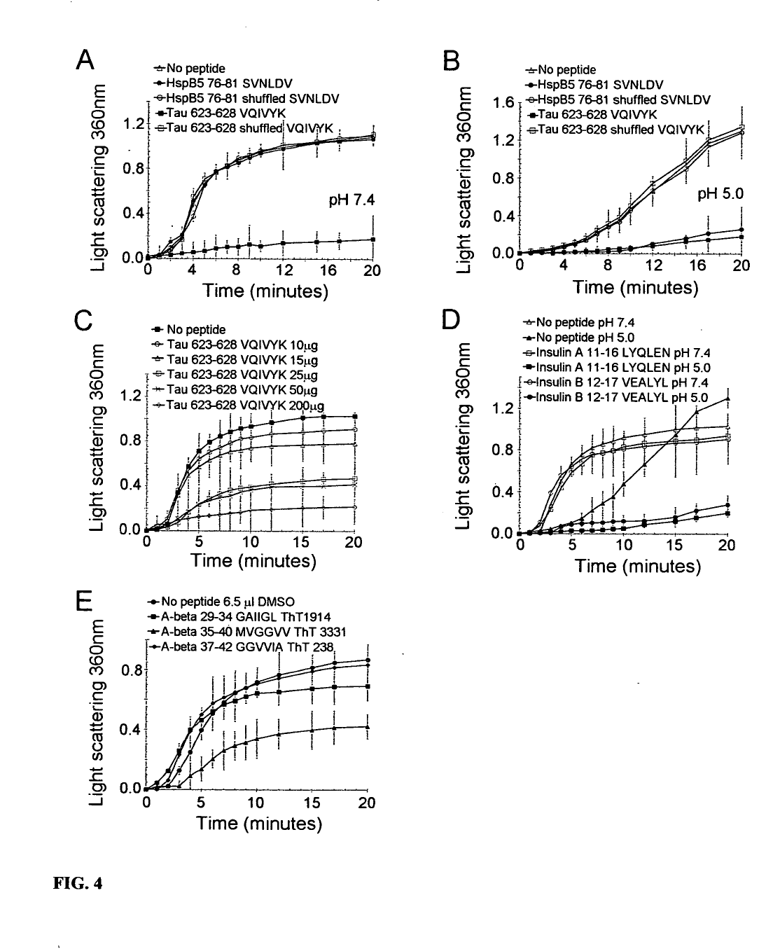

When HspB5 76-81 and Tau 623-628 were assayed for their ability to inhibit insulin aggregation, a clear distinction was apparent. The Tau peptide was a potent inhibitor at both pH 7.4 and pH 5, but the HspB5 peptide was only effective at pH 5 (Panels A and B of FIG. 4), which correlated with the pH dependence of ThT staining (Panels A and D of FIG. 3). The shuffled analogs of both peptides did not inhibit at either both pH 7.4 or pH 5. The inhibition with the Tau peptide was titratable, which allowed the calculation of an IC50 value for the Tau peptide of approximately 20 mg (Panel C of FIG. 4). The inhibition was dose dependent and both inhibitors delayed the appearance of the light scattering and consequently inhibited the nucleation step of the aggregation process (Panels A-C of FIG. 4).

The anionic peptide HspB5 76-81 is a molecular chaperone only at pH4 and not in more basic solutions, consistent with the observed ThT staining. Tau 623-628 inhibited insulin aggregation at pH 4 and pH 7.4. (Panel E of FIG. 4) Similarly, anionic peptides insulin alpha chain 11-16 and insulin beta chain 12-17 are molecular chaperones at pH 4, but not at pH 7.4.

5. Binding of Plasma Proteins by Amyloid Fibrils.

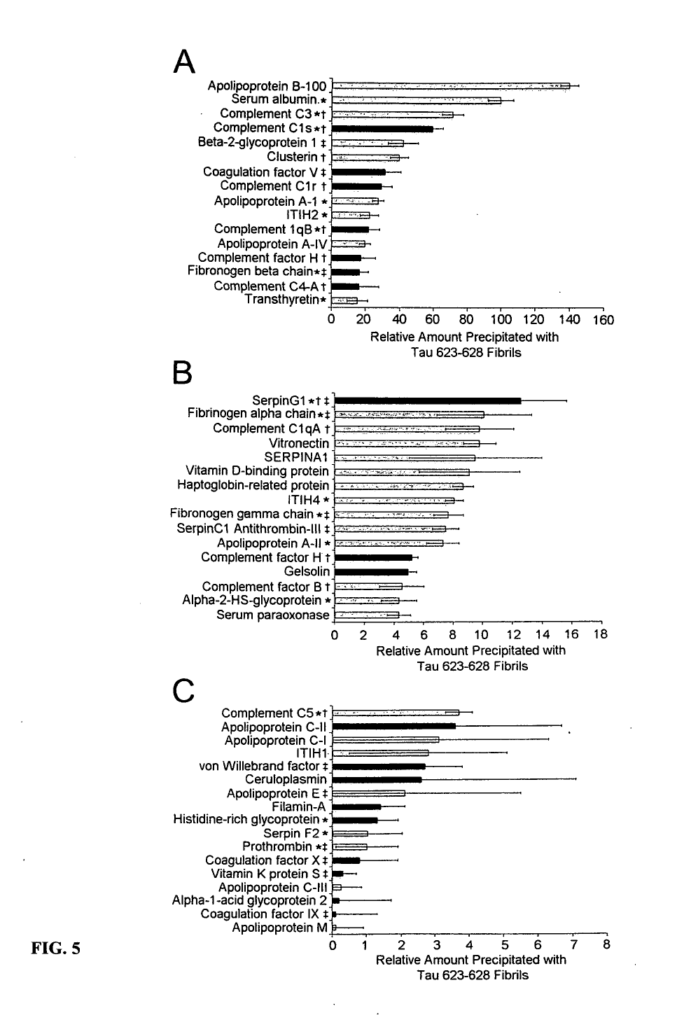

A comparison of the results from Experiments 1-4 showed that molecular chaperone function does not necessarily correlate with therapeutic activity; certain hexamers are effective therapeutics (see Experiment 1) but they do not exhibit molecular chaperone function. To determine whether the therapeutic compounds bind to specific plasma proteins, and accordingly can function as a therapeutic complex, Tau peptide was biotinylated and mixed with the unmodified hexamer to create a fibril that could be precipitated with streptavidin. Incubation of the two peptide mixtures with plasma from three separate MS patients, with subsequent precipitation, elution, tryptic cleavage, and mass spectral analyses, as described in the methods, resulted in the identification of 49 proteins whose relative concentrations were enhanced relative to the set of proteins identified when the streptavidin resin alone was used (see FIG. 5).

Forty-one of the proteins (84%) were among those identified by precipitation with the small heat shock protein, HspB5. See, Rothbard, J. B. et al. Therapeutic effects of systemic administration of chaperone alphaB-crystallin associated with binding proinflammatory plasma proteins. J Biol Chem 287, 9708-9721, doi:M111.337691 [pii] 10.1074/jbc.M111.337691 (2012). An extraordinarily high percentage of the proteins precipitated by the Tau fibril were members of the acute phase response (19/49, 39%), coagulation (13/49, 27%) or complement (11/49, 23%) pathways, which overall represented 33 of the 49 proteins (67%) precipitated. Strong evidence that the set of proteins constituted biologically relevant ligands was the presence of five proteins known to bind amyloid fibrils: Apolipoprotein A-I, A-IV, clusterin 20, apolipoproteins E 19, and transthyretin21. Many of the molecules described here also were identified in laser capture microdissected lesions from MS patients including Apolipoproteins AI, A2, B-100, and E, HspB5, tau, and amyloid precursor protein. See, Han, M. H. et al. Proteomic analysis of active multiple sclerosis lesions reveals therapeutic targets. Nature 451, 1076-1081 (2008). In addition, many of the molecules identified here also are known to modulate EAE. Earlier studies have shown that inhibition of angiotensin converting enzyme or angiotensin receptor, and inhibition of prothrombin all potently inhibit EAE. See Id. Also see, Platten, M. et al. Blocking angiotensin-converting enzyme induces potent regulatory T cells and modulates TH1- and TH17-mediated autoimmunity. Proc Natl Acad Sci USA 106, 14948-14953, doi:0903958106 [pii] 10.1073/pnas.0903958106 (2009). Interestingly, mice with genes encoding apolipoprotein E 23, tau, HspB5 1, and APP 24 individually knocked out all exhibit exacerbated EAE.

6. Therapeutic Effect of the Amyloid Fibrils does not Invoke Stereospecific Cell Surface Receptor

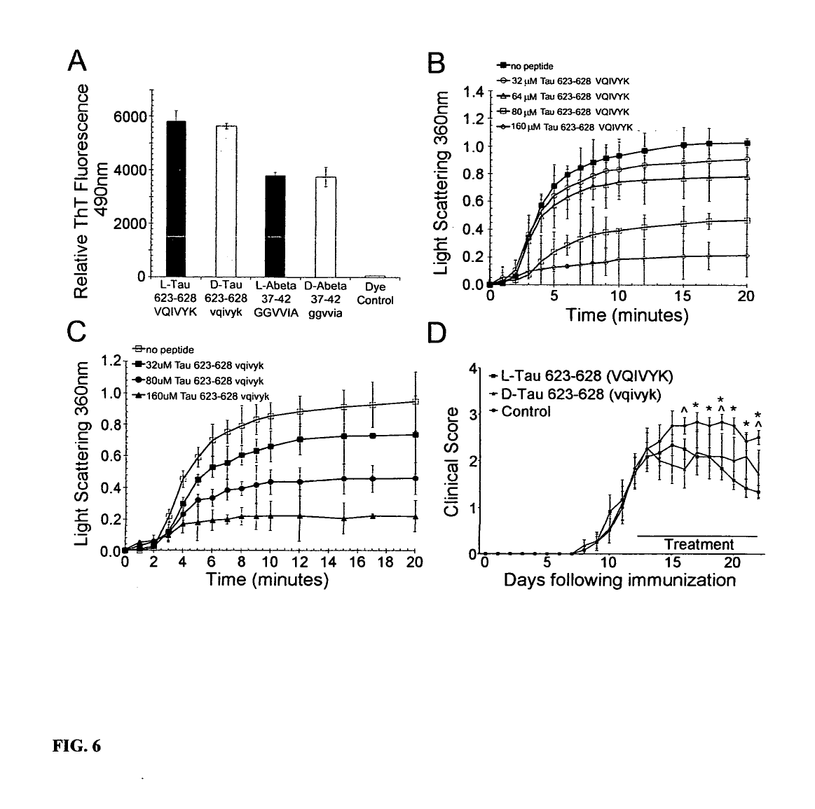

The comparative ability of Tau 623-628 (Ac VQIVYK CONH2)(SEQ ID NO: 123) and A beta 35-40 (Ac MVGGVV CONH2)(SEQ ID NO: 220) composed of L or D-amino acids to form amyloid fibrils was shown to be equivalent, within error, as defined by staining with Thioflavin T (FIG. 6a). The resultant stereoisomeric fibrils composed of the Tau peptide also inhibited insulin aggregation with very similar IC50 values (FIG. 6b). When used as therapeutic agents for EAE, equivalent reduction of paralytic symptoms were observed using peptides composed of D- or L-amino acids. This result has several important implications. The first is that the mechanism(s) will not involve binding to a stereospecific receptor. The second issue is that a common shared feature between the two stereoisomers is their molecular chaperone function. Not surprisingly, proteolytic cleavage of the peptides does not appear to be a major factor in limiting their mode of action, and lastly the set of possible relevant sequences has doubled with the shared activity of the peptides composed of D and L amino acids.

With respect the experimental results depicted in FIG. 6: A) L- and D-tau 623-628 and L- and D-Aβ 37-42 (200 μg/50 μl) were each added to 100 μl of 100 mM MES pH 7.4 and incubated at 37° C. ThT (10 μl of 10 μM solution) was added and the resultant fluorescence at 485 nm from excitation at 440 nm was measured using a fluorescent plate reader. The levels of ThT were similar in both sets of hexapeptides. B) L-tau and C) D-tau 623-628 inhibit insulin aggregation in a dose dependent manner with similar IC50 concentrations. D) Groups of ten mice were injected daily intraperitoneally for 10 days with 1 μg of L-tau or D-tau hexapeptides beginning at onset of hindlimb weakness. PBS was injected in littermates as control. Bar represents the duration of the treatment. Values in graph represent mean+/−S.E.M. *p<0.05 for L-tau and ̂p<0.05 for D-tau by Mann-Whitney U test.

1. A composition for treatment of one or more inflammatory conditions in a mammal wherein the composition comprises one or more hexapeptides comprising amino acids of an L-configuration, D-configuration or a mixture of configurations having free energy of formation of −23 kcal/mol or less when the hexapeptide sequence in subjected to the Rosetta-Profile algorithm.

2. The composition according to claim 1, wherein the hexapeptides are selected from a group consisting of:

| | |
|---|---|
| SVNVDL | (SEQ ID NO: 1); |
| SLNVDV | (SEQ ID NO: 2); |
| SVDVNL | (SEQ ID NO: 3); |
| DLSVVL | (SEQ ID NO: 4); |
| SVNLDV | (SEQ ID NO: 5); |
| SVVNDV | (SEQ ID NO: 6); |
| DVSLVN | (SEQ ID NO: 7); |
| DVSVLN | (SEQ ID NO: 8); |
| SDLVNV | (SEQ ID NO: 9); |
| SLNVVS | (SEQ ID NO: 10); |
| LNVDSV | (SEQ ID NO: 11); |
| NDLSVV | (SEQ ID NO: 12); |
| VDNLVS | (SEQ ID NO: 13); |
| VNDVSL | (SEQ ID NO: 14); |
| VSDNVL | (SEQ ID NO: 15); |
| LNDVVS | (SEQ ID NO: 16); |
| LSVDVN | (SEQ ID NO: 17); |
| NSVDLV | (SEQ ID NO: 18); |
| VDVLNS | (SEQ ID NO: 19); |
| VNDSVL | (SEQ ID NO: 20); |
| VNVSLD | (SEQ ID NO: 21); |
| LDVNSV | (SEQ ID NO: 22); |
| LNVVDS | (SEQ ID NO: 23); |
| LSDVVN | (SEQ ID NO: 24); |
| LVVNDS | (SEQ ID NO: 25); |
| NSLDVV | (SEQ ID NO: 26); |
| VLVDNS | (SEQ ID NO: 27); |
| VNLSDV | (SEQ ID NO: 28); |
| VNVDLS | (SEQ ID NO: 29); |
| DNVSVD | (SEQ ID NO: 30); |
| LSDNVV | (SEQ ID NO: 31); |
| LSVVDN | (SEQ ID NO: 32); |
| VLDVSN | (SEQ ID NO: 33); |
| VNDLVS | (SEQ ID NO: 34); |
| VNVSDL | (SEQ ID NO: 35); |
| VVLSDN | (SEQ ID NO: 36); |
| LVSVNL | (SEQ ID NO: 37); |
| LVNVSV | (SEQ ID NO: 38); |
| SLNVSV | (SEQ ID NO: 39); |
| LVSVNS | (SEQ ID NO: 40); |
| SVDVNV | (SEQ ID NO: 41); |
| LVVSVL | (SEQ ID NO: 42); |
| VNLVVS | (SEQ ID NO: 43); |
| SNLVSV | (SEQ ID NO: 44); |
| SVNVLS | (SEQ ID NO: 45); |
| VLVSVL | (SEQ ID NO: 46); |
| LVNVSL | (SEQ ID NO: 47); |
| SVNVDS | (SEQ ID NO: 48); |
| VLSVNV | (SEQ ID NO: 49); |
| NLVVSV | (SEQ ID NO: 50); |
| SVVLNV | (SEQ ID NO: 51); |
| LSVVNL | (SEQ ID NO: 52); |
| SNLVVS | (SEQ ID NO: 53); |
| SVVLDV | (SEQ ID NO: 54); |
| LVSLNV | (SEQ ID NO: 55); |
| VSLNVV | (SEQ ID NO: 56); |
| VKVQIY | (SEQ ID NO: 57); |
| QVVIYK | (SEQ ID NO: 58); |
| KVIQVY | (SEQ ID NO: 59); |
| VYVKIY | (SEQ ID NO: 60); |
| QIVVYK | (SEQ ID NO: 61); |
| QVIKVY | (SEQ ID NO: 62); |
| VQVKIY | (SEQ ID NO: 63); |
| QVVKIY | (SEQ ID NO: 64); |
| QVIVYK | (SEQ ID NO: 65); |
| QIVKVY | (SEQ ID NO: 66); |
| QKIVVY | (SEQ ID NO: 67); |
| QKVVYI | (SEQ ID NO: 68); |
| KVQVYI | (SEQ ID NO: 69); |
| QVVKYI | (SEQ ID NO: 70); |
| KVQIYV | (SEQ ID NO: 71); |
| VKIQVY | (SEQ ID NO: 72); |
| VIQKVY | (SEQ ID NO: 73); |
| KVVIYK | (SEQ ID NO: 74); |
| QIVKYV | (SEQ ID NO: 75); |
| QKVIYV | (SEQ ID NO: 76); |
| KQVVIY | (SEQ ID NO: 77); |
| KIQVYV | (SEQ ID NO: 78); |
| KVYVQI | (SEQ ID NO: 79); |
| VVQIYK | (SEQ ID NO: 80); |
| KQVIVY | (SEQ ID NO: 81); |
| VQIKVY | (SEQ ID NO: 82); |
| QKIVYV | (SEQ ID NO: 83); |
| VIQVYK | (SEQ ID NO: 84); |
| KVVIQY | (SEQ ID NO: 85); |
| KVQVIY | (SEQ ID NO: 86); |
| QYVVIK | (SEQ ID NO: 87); |
| YVVIQK | (SEQ ID NO: 88); |
| KIVVQY | (SEQ ID NO: 89); |
| YQVIVK | (SEQ ID NO: 90); |
| KIYVQV | (SEQ ID NO: 91); |
| KVVIYQ | (SEQ ID NO: 92); |
| KYVVQI | (SEQ ID NO: 93); |
| YQIVVK | (SEQ ID NO: 94); |
| YVVIQY | (SEQ ID NO: 95); |
| KQVIYV | (SEQ ID NO: 96); |
| KYVQVI | (SEQ ID NO: 97); |
| KYVVIQ | (SEQ ID NO: 98); |
| QKVVIY | (SEQ ID NO: 99); |
| QYVIVK | (SEQ ID NO: 100); |
| IVQKVY | (SEQ ID NO: 101); |
| KYIVVQ | (SEQ ID NO: 102); |
| QVIKYV | (SEQ ID NO: 103); |
| YIVVQK | (SEQ ID NO: 104); |
| YQVVIK | (SEQ ID NO: 105); |
| IKVQVY | (SEQ ID NO: 106); |
| KVVQYI | (SEQ ID NO: 107); |
| KYVQIV | (SEQ ID NO: 108); |
| VKQVYI | (SEQ ID NO: 109); |
| VQVIYK | (SEQ ID NO: 110); |
| VVIQKY | (SEQ ID NO: 111); |
| IQVVYK | (SEQ ID NO: 112); |
| KIVVYQ | (SEQ ID NO: 113); |
| KYQVIV | (SEQ ID NO: 114); |
| KYVIVQ | (SEQ ID NO: 115); |
| VVYIQK | (SEQ ID NO: 116); |
| IVQVYK | (SEQ ID NO: 117); |
| KQIVYV | (SEQ ID NO: 118); |
| KYVIQV | (SEQ ID NO: 119); |
| QKVIVY | (SEQ ID NO: 120); |
| QYIKVV | (SEQ ID NO: 121); |
| QYIVKV | (SEQ ID NO: 122); |
| VQIVYK | (SEQ ID NO: 123); |
| VVQKYI | (SEQ ID NO: 124); |
| VYQIVK | (SEQ ID NO: 125); |
| YVQVIK | (SEQ ID NO: 126); |
| QIVYVK | (SEQ ID NO: 127); |
| IKVYQV | (SEQ ID NO: 128); |
| KVVYIQ | (SEQ ID NO: 129); |
| VQKYIV | (SEQ ID NO: 130); |
| VQKYVI | (SEQ ID NO: 131); |
| KVVQIY | (SEQ ID NO: 132); |
| YVIKIY | (SEQ ID NO: 133); |
| QVIVYV | (SEQ ID NO: 134); |
| VVIKVY | (SEQ ID NO: 135); |
| KVVIYI | (SEQ ID NO: 136); |
| VYVVIK | (SEQ ID NO: 137); |
| YIVQVY | (SEQ ID NO: 138); |
| YVIQVY | (SEQ ID NO: 139); |
| YVIVVY | (SEQ ID NO: 140); |
| KVIVYI | (SEQ ID NO: 141); |
| YVIKVY | (SEQ ID NO: 142); |
| VQVIVK | (SEQ ID NO: 143); |
| YVVKIY | (SEQ ID NO: 144); |
| YVVQVI | (SEQ ID NO: 145); |
| QIVVYQ | (SEQ ID NO: 146); |
| VVQIVK | (SEQ ID NO: 147); |
| VQIVVK | (SEQ ID NO: 148); |
| VIVVYK | (SEQ ID NO: 149); |
| IVQVYI | (SEQ ID NO: 150); |
| YVVIQV | (SEQ ID NO: 151); |
| VYQVVI | (SEQ ID NO: 152); |
| KYIQVY | (SEQ ID NO: 153); |
| KYVVIY | (SEQ ID NO: 154); |
| KYVQIY | (SEQ ID NO: 155); |
| VVQVIK | (SEQ ID NO: 156); |
| QKIVVK | (SEQ ID NO: 157); |
| KYVIVY | (SEQ ID NO: 158); |
| KQVVIK | (SEQ ID NO: 159); |
| YVQIYV | (SEQ ID NO: 160); |
| YKVQVY | (SEQ ID NO: 161); |
| IKVQIY | (SEQ ID NO: 162); |
| KIVVQK | (SEQ ID NO: 163); |
| VIKVVI | (SEQ ID NO: 164); |
| VYIKVV | (SEQ ID NO: 165); |
| VYIVQV | (SEQ ID NO: 166); |
| IVYVQI | (SEQ ID NO: 167); |
| KVQIYK | (SEQ ID NO: 168); |
| IYVIVY | (SEQ ID NO: 169); |
| VIYVIV | (SEQ ID NO: 170); |
| KYQVYI | (SEQ ID NO: 171); |
| VIQKVV | (SEQ ID NO: 172); |
| VKIVYV | (SEQ ID NO: 173); |
| VKQIVV | (SEQ ID NO: 174); |
| YIVKQY | (SEQ ID NO: 175); |
| YIVVQY | (SEQ ID NO: 176); |
| YKIQVY | (SEQ ID NO: 177); |
| YQVVIY | (SEQ ID NO: 178); |
| YVVKQY | (SEQ ID NO: 179); |
| VIVKVQ | (SEQ ID NO: 180); |
| QKVVIK | (SEQ ID NO: 181); |
| GMVVVG | (SEQ ID NO: 182); |
| GVVVMG | (SEQ ID NO: 183); |
| GVVMVG | (SEQ ID NO: 184); |
| GGVVVM | (SEQ ID NO: 185); |
| GVVGVM | (SEQ ID NO: 186); |
| GVVVGM | (SEQ ID NO: 187); |
| GVMVVG | (SEQ ID NO: 188); |
| GGVVMV | (SEQ ID NO: 189); |
| GGVMVV | (SEQ ID NO: 190); |
| GMVVGV | (SEQ ID NO: 191); |
| GVVMGV | (SEQ ID NO: 192); |
| GMVGVV | (SEQ ID NO: 193); |
| GVMVGV | (SEQ ID NO: 194); |
| GVVGMV | (SEQ ID NO: 195); |
| GGMVVV | (SEQ ID NO: 196); |
| GVGVMV | (SEQ ID NO: 197); |
| GVGVVM | (SEQ ID NO: 198); |
| MVVVGM | (SEQ ID NO: 199); |
| GMGVVV | (SEQ ID NO: 200); |
| MVVVGG | (SEQ ID NO: 201); |
| GVGMVV | (SEQ ID NO: 202); |
| GVVGGV | (SEQ ID NO: 203); |
| MVGVVG | (SEQ ID NO: 204); |
| VVMVGG | (SEQ ID NO: 205); |
| MGVVVG | (SEQ ID NO: 206); |
| MVGVGV | (SEQ ID NO: 207); |
| VGVMVG | (SEQ ID NO: 208); |
| MVVGVG | (SEQ ID NO: 209); |
| VVGVMG | (SEQ ID NO: 210); |
| VMGVVG | (SEQ ID NO: 211); |
| VGVGVM | (SEQ ID NO: 212); |
| VMVGVG | (SEQ ID NO: 213); |
| MGGVVV | (SEQ ID NO: 214); |
| VGMVVG | (SEQ ID NO: 215); |
| VVGVGM | (SEQ ID NO: 216); |
| VGGVVM | (SEQ ID NO: 217); |
| VVGMVG | (SEQ ID NO: 218); |
| VGVVMG | (SEQ ID NO: 219); |
| MVGGVV | (SEQ ID NO: 220); |
| VGMVGV | (SEQ ID NO: 221); |
| VMVVGG | (SEQ ID NO: 222); |
| VVGGVM | (SEQ ID NO: 223); |
| VGGVMV | (SEQ ID NO: 224); |
| VVVGMG | (SEQ ID NO: 225); |
| VVGMGV | (SEQ ID NO: 226); |
| MGVGVV | (SEQ ID NO: 227); |
| VGVVGM | (SEQ ID NO: 228); |
| VGGMVV | (SEQ ID NO: 229); |
| VVVMGG | (SEQ ID NO: 230); |
| VVMGGV | (SEQ ID NO: 231); |
| VVVGGM | (SEQ ID NO: 232); |
| VVGGMV | (SEQ ID NO: 233); |
| VGMGVV | (SEQ ID NO: 234); |
| VMGGVV | (SEQ ID NO: 235); |
| VGGVGV | (SEQ ID NO: 236); |
| VGVMGV | (SEQ ID NO: 237); |
| VGVGMV | (SEQ ID NO: 238); |
| and | |
| VMVGGV | (SEQ ID NO: 239); |
| ANSTSV | (SEQ ID NO: 240); |
| ANSVSG | (SEQ ID NO: 241); |
| ANSVSS | (SEQ ID NO: 242); |
| AQNSNV | (SEQ ID NO: 243); |
| AQNVNS | (SEQ ID NO: 244); |
| AQNVTS | (SEQ ID NO: 245); |
| AQSQSV | (SEQ ID NO: 246); |
| AQSSSV | (SEQ ID NO: 247); |
| AQSTSV | (SEQ ID NO: 248); |
| AQSVNS | (SEQ ID NO: 249); |
| AQSVQS | (SEQ ID NO: 250); |
| AQSVSQ | (SEQ ID NO: 251); |
| AQSVSS | (SEQ ID NO: 252); |
| AQSVST | (SEQ ID NO: 253); |
| ASNNNV | (SEQ ID NO: 254); |
| ASNQNQ | (SEQ ID NO: 255); |
| ASNQNV | (SEQ ID NO: 256); |
| ASNQTQ | (SEQ ID NO: 257); |
| ASNSNV | (SEQ ID NO: 258); |
| ASNSTV | (SEQ ID NO: 259); |
| ASNTNS | (SEQ ID NO: 260); |
| ASNTNV | (SEQ ID NO: 261); |
| ASNTSV | (SEQ ID NO: 262); |
| ASNVNG | (SEQ ID NO: 263); |
| ASNVNQ | (SEQ ID NO: 264); |
| ASNVNS | (SEQ ID NO: 265); |
| ASNVNT | (SEQ ID NO: 266); |
| ASNVTG | (SEQ ID NO: 267); |
| ASNVTT | (SEQ ID NO: 268); |
| ASSNSV | (SEQ ID NO: 269); |
| ASSVSG | (SEQ ID NO: 270); |
| ASSVSN | (SEQ ID NO: 271); |
| ATNVNS | (SEQ ID NO: 272); |
| ATNVTS | (SEQ ID NO: 273); |
| ATSQSQ | (SEQ ID NO: 274); |
| ATSQSV | (SEQ ID NO: 275); |
| ATSTSG | (SEQ ID NO: 276); |
| ATSTSV | (SEQ ID NO: 277); |
| ATSVSG | (SEQ ID NO: 278); |
| ATSVSS | (SEQ ID NO: 279); |
| AVNQNS | (SEQ ID NO: 280); |
| AVNQSQ | (SEQ ID NO: 281); |
| AVNSNG | (SEQ ID NO: 282); |
| AVNSNS | (SEQ ID NO: 283); |
| AVNSNT | (SEQ ID NO: 284); |
| AVNTNS | (SEQ ID NO: 285); |
| AVSNSG | (SEQ ID NO: 286); |
| AVSNSS | (SEQ ID NO: 287); |
| AVSQNQ | (SEQ ID NO: 288); |
| AVSQSG | (SEQ ID NO: 289); |
| AVSQSQ | (SEQ ID NO: 290); |
| AVSQTQ | (SEQ ID NO: 291); |
| AVSSNQ | (SEQ ID NO: 292); |
| AVSSNS | (SEQ ID NO: 293); |
| AVSSSQ | (SEQ ID NO: 294); |
| AVSTSG | (SEQ ID NO: 295); |
| GANTVS | (SEQ ID NO: 296); |
| GAQTSS | (SEQ ID NO: 297); |
| GASNQS | (SEQ ID NO: 298); |
| GASQQS | (SEQ ID NO: 299); |
| GASSQQ | (SEQ ID NO: 300) |
| GGQVTS | (SEQ ID NO: 301); |
| GGSNQV | (SEQ ID NO: 302); |
| GNNVQS | (SEQ ID NO: 303); |
| GNQVTS | (SEQ ID NO: 304); |
| GNSNQV | (SEQ ID NO: 305); |
| GNSQQQ | (SEQ ID NO: 306); |
| GNSQQS | (SEQ ID NO: 307); |
| GNSQQV | (SEQ ID NO: 308); |
| GNSSTV | (SEQ ID NO: 309); |
| GNSTQS | (SEQ ID NO: 310); |
| GNSTQV | (SEQ ID NO: 311); |
| GNSTVS | (SEQ ID NO: 312); |
| GNSVQS | (SEQ ID NO: 313); |
| GNSVSS | (SEQ ID NO: 314); |
| GNSVST | (SEQ ID NO: 315); |
| GNSVTS | (SEQ ID NO: 316); |
| GQNTVS | (SEQ ID NO: 317); |
| GQNVAS | (SEQ ID NO: 318); |
| GQNVQS | (SEQ ID NO: 319); |
| GQNVSS | (SEQ ID NO: 320); |
| GQNVTS | (SEQ ID NO: 321); |
| GQQQSQ | (SEQ ID NO: 322); |
| GQQTSS | (SEQ ID NO: 323); |
| GQQTSV | (SEQ ID NO: 324); |
| GQQVAS | (SEQ ID NO: 325); |
| GQQVNS | (SEQ ID NO: 326); |
| GQQVQS | (SEQ ID NO: 327); |
| GQQVSG | (SEQ ID NO: 328); |
| GQQVSQ | (SEQ ID NO: 329); |
| GQQVSS | (SEQ ID NO: 330); |
| GQQVST | (SEQ ID NO: 331); |
| GQQVTS | (SEQ ID NO: 332); |
| GQSGQV | (SEQ ID NO: 333); |
| GQSNQA | (SEQ ID NO: 334); |
| GQSNQS | (SEQ ID NO: 335); |
| GQSNQV | (SEQ ID NO: 336); |
| GQSQAQ | (SEQ ID NO: 337); |
| GQSQQQ | (SEQ ID NO: 338); |
| GQSQQS | (SEQ ID NO: 339); |
| GQSQSQ | (SEQ ID NO: 340); |
| GQSSQQ | (SEQ ID NO: 341); |
| GQSSQS | (SEQ ID NO: 342); |
| GQSSQV | (SEQ ID NO: 343); |
| GQSTQS | (SEQ ID NO: 344); |
| GQSTQV | (SEQ ID NO: 345); |
| GQSVAG | (SEQ ID NO: 346); |
| GQSVAQ | (SEQ ID NO: 347); |
| GQSVAS | (SEQ ID NO: 348); |
| GQSVQN | (SEQ ID NO: 349); |
| GQSVQQ | (SEQ ID NO: 350); |
| GQSVQS | (SEQ ID NO: 351); |
| GQSVSG | (SEQ ID NO: 352); |
| GQSVSN | (SEQ ID NO: 353); |
| GQSVSQ | (SEQ ID NO: 354); |
| GQSVSS | (SEQ ID NO: 355); |
| GQSVST | (SEQ ID NO: 356); |
| GQSVTS | (SEQ ID NO: 357); |
| GSNQQV | (SEQ ID NO: 358); |
| GSNQVQ | (SEQ ID NO: 359); |
| GSNSTV | (SEQ ID NO: 360); |
| GSNSVQ | (SEQ ID NO: 361); |
| GSNSVT | (SEQ ID NO: 362); |
| GSNTAV | (SEQ ID NO: 363); |
| GSNTQV | (SEQ ID NO: 364); |
| GSNTVA | (SEQ ID NO: 365); |
| GSNTVS | (SEQ ID NO: 366); |
| GSNVAS | (SEQ ID NO: 367); |
| GSNVQQ | (SEQ ID NO: 368); |
| GSNVQS | (SEQ ID NO: 369); |
| GSNVQT | (SEQ ID NO: 370); |
| GSNVTS | (SEQ ID NO: 371); |
| GSQQSV | (SEQ ID NO: 372); |
| GSQQTQ | (SEQ ID NO: 373); |
| GSQQTV | (SEQ ID NO: 374); |
| GSQTSS | (SEQ ID NO: 375); |
| GSQTSV | (SEQ ID NO: 376); |
| GSQVAS | (SEQ ID NO: 377); |
| GSQVNS | (SEQ ID NO: 378); |
| GSQVQS | (SEQ ID NO: 379); |
| GSQVSS | (SEQ ID NO: 380); |
| GSQVST | (SEQ ID NO: 381); |
| GSQVTG | (SEQ ID NO: 382); |
| GSQVTS | (SEQ ID NO: 383); |
| GSSAQS | (SEQ ID NO: 384); |
| GSSGQV | (SEQ ID NO: 385); |
| GSSNQA | (SEQ ID NO: 386); |
| GSSNQT | (SEQ ID NO: 387); |
| GSSNQV | (SEQ ID NO: 388); |
| GSSNSV | (SEQ ID NO: 389); |
| GSSNTV | (SEQ ID NO: 390); |
| GSSNVT | (SEQ ID NO: 391); |
| GSSQAQ | (SEQ ID NO: 392); |
| GSSQQA | (SEQ ID NO: 393); |
| GSSQQV | (SEQ ID NO: 394); |
| GSSQSQ | (SEQ ID NO: 395); |
| GSSSQQ | (SEQ ID NO: 396); |
| GSSSQS | (SEQ ID NO: 397); |
| GSSSQV | (SEQ ID NO: 398); |
| GSSTQA | (SEQ ID NO: 399); |
| GSSTQG | (SEQ ID NO: 400); |
| GSSTQT | (SEQ ID NO: 401); |
| GSSTQV | (SEQ ID NO: 402); |
| GSSTSV | (SEQ ID NO: 403); |
| GSSTVN | (SEQ ID NO: 404); |
| GSSVNS | (SEQ ID NO: 405); |
| GSSVQN | (SEQ ID NO: 406); |
| GSSVQT | (SEQ ID NO: 407); |
| GSSVSG | (SEQ ID NO: 408); |
| GSSVSS | (SEQ ID NO: 409); |
| GSSVST | (SEQ ID NO: 410); |
| GSSVTN | (SEQ ID NO: 411); |
| GTNSSV | (SEQ ID NO: 412); |
| GTNSVS | (SEQ ID NO: 413); |
| GTNTVS | (SEQ ID NO: 414); |
| GTNVQS | (SEQ ID NO: 415); |
| GTNVSS | (SEQ ID NO: 416); |
| GTNVTS | (SEQ ID NO: 417); |
| GTQQSQ | (SEQ ID NO: 418); |
| GTQSTS | (SEQ ID NO: 419); |
| GTQSTV | (SEQ ID NO: 420); |
| GTQTSA | (SEQ ID NO: 421); |
| GTQTSV | (SEQ ID NO: 422); |
| GTQVQS | (SEQ ID NO: 423); |
| GTQVSG | (SEQ ID NO: 424); |
| GTQVSN | (SEQ ID NO: 425); |
| GTQVSS | (SEQ ID NO: 426); |
| GTQVST | (SEQ ID NO: 427); |
| GTSAQS | (SEQ ID NO: 428); |
| GTSGQV | (SEQ ID NO: 429); |
| GTSNQA | (SEQ ID NO: 430); |
| GTSNQS | (SEQ ID NO: 431); |
| GTSNQT | (SEQ ID NO: 432); |
| GTSNQV | (SEQ ID NO: 433); |
| GTSNSV | (SEQ ID NO: 434); |
| GTSNVS | (SEQ ID NO: 435); |
| GTSQQA | (SEQ ID NO: 436); |
| GTSQQS | (SEQ ID NO: 437); |
| GTSQQV | (SEQ ID NO: 438); |
| GTSQSQ | (SEQ ID NO: 439); |
| GTSQSV | (SEQ ID NO: 440); |
| GTSSNV | (SEQ ID NO: 441); |
| GTSTQA | (SEQ ID NO: 442); |
| GTSTQS | (SEQ ID NO: 443); |
| GTSTQT | (SEQ ID NO: 444); |
| GTSTQV | (SEQ ID NO: 445); |
| GTSTSV | (SEQ ID NO: 446); |
| GTSVAG | (SEQ ID NO: 447); |
| GTSVAS | (SEQ ID NO: 448); |
| GTSVNS | (SEQ ID NO: 449); |
| GTSVNT | (SEQ ID NO: 450); |
| GTSVQG | (SEQ ID NO: 451); |
| GTSVQN | (SEQ ID NO: 452); |
| GTSVQQ | (SEQ ID NO: 453); |
| GTSVQS | (SEQ ID NO: 454); |
| GTSVQT | (SEQ ID NO: 455); |
| GTSVSG | (SEQ ID NO: 456); |
| GTSVSN | (SEQ ID NO: 457); |
| GTSVSS | (SEQ ID NO: 458); |
| GTSVST | (SEQ ID NO: 459); |
| GTSVTS | (SEQ ID NO: 460); |
| GVNSQS | (SEQ ID NO: 461); |
| GVNSST | (SEQ ID NO: 462); |
| GVNSTS | (SEQ ID NO: 463); |
| GVNTQS | (SEQ ID NO: 464); |
| GVNTSS | (SEQ ID NO: 465); |
| GVQNTS | (SEQ ID NO: 466); |
| GVQQSQ | (SEQ ID NO: 467); |
| GVQSNQ | (SEQ ID NO: 468); |
| GVQSNS | (SEQ ID NO: 469); |
| GVQSSG | (SEQ ID NO: 470); |
| GVQSSQ | (SEQ ID NO: 471); |
| GVQSTS | (SEQ ID NO: 472); |
| GVQSTT | (SEQ ID NO: 473); |
| GVQTNS | (SEQ ID NO: 474); |
| GVQTQS | (SEQ ID NO: 475); |
| GVQTSG | (SEQ ID NO: 476); |
| GVQTSS | (SEQ ID NO: 477); |
| GVSGQG | (SEQ ID NO: 478); |
| GVSGQT | (SEQ ID NO: 479); |
| GVSNAS | (SEQ ID NO: 480); |
| GVSNNV | (SEQ ID NO: 481); |
| GVSNQG | (SEQ ID NO: 482); |
| GVSNQN | (SEQ ID NO: 483); |
| GVSNQQ | (SEQ ID NO: 484); |
| GVSNQS | (SEQ ID NO: 485); |
| GVSNQT | (SEQ ID NO: 486); |
| GVSNSS | (SEQ ID NO: 487); |
| GVSNST | (SEQ ID NO: 488); |
| GVSNTS | (SEQ ID NO: 489); |
| GVSQAQ | (SEQ ID NO: 490); |
| GVSQNQ | (SEQ ID NO: 491); |
| GVSQNS | (SEQ ID NO: 492); |
| GVSQQG | (SEQ ID NO: 493); |
| GVSQQS | (SEQ ID NO: 494); |
| GVSQQT | (SEQ ID NO: 495); |
| GVSQSG | (SEQ ID NO: 496); |
| GVSQSQ | (SEQ ID NO: 497); |
| GVSQSS | (SEQ ID NO: 498); |
| GVSSAQ | (SEQ ID NO: 499); |
| GVSSAS | (SEQ ID NO: 500); |
| GVSSGQ | (SEQ ID NO: 501); |
| GVSSNG | (SEQ ID NO: 502); |
| GVSSNQ | (SEQ ID NO: 503); |
| GVSSNS | (SEQ ID NO: 504); |
| GVSSNT | (SEQ ID NO: 505); |
| GVSSQG | (SEQ ID NO: 506); |
| GVSSQN | (SEQ ID NO: 507); |
| GVSSQQ | (SEQ ID NO: 508); |
| GVSSSG | (SEQ ID NO: 509); |
| GVSSSQ | (SEQ ID NO: 510); |
| GVSSSS | (SEQ ID NO: 511); |
| GVSSTN | (SEQ ID NO: 512); |
| GVSTAS | (SEQ ID NO: 513); |
| GVSTNS | (SEQ ID NO: 514); |
| GVSTQG | (SEQ ID NO: 515); |
| GVSTQN | (SEQ ID NO: 516); |
| GVSTQQ | (SEQ ID NO: 517); |
| GVSTQS | (SEQ ID NO: 518); |
| GVSTQT | (SEQ ID NO: 519); |
| GVSTSN | (SEQ ID NO: 520); |
| GVSTSQ | (SEQ ID NO: 521); |
| GVSTSS | (SEQ ID NO: 522); |
| GVSTST | (SEQ ID NO: 523); |
| GVTNSS | (SEQ ID NO: 524); |
| GVTSNS | (SEQ ID NO: 525); |
| GVTSSN | (SEQ ID NO: 526); |
| NGSTSV | (SEQ ID NO: 527); |
| NGSVTS | (SEQ ID NO: 528); |
| NNSVSS | (SEQ ID NO: 529); |
| NQSQSQ | (SEQ ID NO: 530); |
| NQSVSN | (SEQ ID NO: 531); |
| NQSVSQ | (SEQ ID NO: 532); |
| NQSVSS | (SEQ ID NO: 533); |
| NSSNSV | (SEQ ID NO: 534); |
| NSSQSQ | (SEQ ID NO: 535); |
| NSSTVG | (SEQ ID NO: 536); |
| NSSVSG | (SEQ ID NO: 537); |
| NSSVSN | (SEQ ID NO: 538); |
| NSSVSS | (SEQ ID NO: 539); |
| NSSVTG | (SEQ ID NO: 540); |
| NTNVNS | (SEQ ID NO: 541); |
| NTQVSS | (SEQ ID NO: 542); |
| NTSGSV | (SEQ ID NO: 543); |
| NTSQSQ | (SEQ ID NO: 544); |
| NTSTSS | (SEQ ID NO: 545); |
| NTSTSV | (SEQ ID NO: 546); |
| NTSVSS | (SEQ ID NO: 547); |
| NVSGST | (SEQ ID NO: 548); |
| NVSNSG | (SEQ ID NO: 549); |
| NVSQSG | (SEQ ID NO: 550); |
| NVSQSQ | (SEQ ID NO: 551); |
| NVSQSS | (SEQ ID NO: 552); |
| NVSSSG | (SEQ ID NO: 553); |
| NVSSSS | (SEQ ID NO: 554); |
| NVSSTG | (SEQ ID NO: 555); |
| NVSTSN | (SEQ ID NO: 556); |
| NVSTSS | (SEQ ID NO: 557); |
| NVSTST | (SEQ ID NO: 558); |
| QNSTSV | (SEQ ID NO: 559); |
| QNSVSG | (SEQ ID NO: 560); |
| QQSQSQ | (SEQ ID NO: 561); |
| QQSVAG | (SEQ ID NO: 562); |
| QQSVAQ | (SEQ ID NO: 563); |
| QQSVAS | (SEQ ID NO: 564); |
| QQSVAT | (SEQ ID NO: 565); |
| QQSVSG | (SEQ ID NO: 566); |
| QQSVSN | (SEQ ID NO: 567); |
| QQSVSS | (SEQ ID NO: 568); |
| QSNQVQ | (SEQ ID NO: 569); |
| QSNTAV | (SEQ ID NO: 570); |
| QSNTNV | (SEQ ID NO: 571); |
| QSSNSV | (SEQ ID NO: 572); |
| QSSQAQ | (SEQ ID NO: 573); |
| QSSQSQ | (SEQ ID NO: 574); |
| QSSQSV | (SEQ ID NO: 575); |
| QSSTSV | (SEQ ID NO: 576); |
| QSSVSG | (SEQ ID NO: 577); |
| QSSVSN | (SEQ ID NO: 578); |
| QSSVSQ | (SEQ ID NO: 579); |
| QSSVSS | (SEQ ID NO: 580); |
| QSSVTG | (SEQ ID NO: 581); |
| QSSVTS | (SEQ ID NO: 582); |
| QTSQSQ | (SEQ ID NO: 583); |
| QTSSSQ | (SEQ ID NO: 584); |
| QTSSSV | (SEQ ID NO: 585); |
| QTSVAG | (SEQ ID NO: 586); |
| QTSVAS | (SEQ ID NO: 587); |
| QTSVSG | (SEQ ID NO: 588); |
| QTSVSN | (SEQ ID NO: 589); |
| QTSVSS | (SEQ ID NO: 590); |
| QTSVST | (SEQ ID NO: 591); |
| QTSVTS | (SEQ ID NO: 592); |
| QVSNSG | (SEQ ID NO: 593); |
| QVSNSS | (SEQ ID NO: 594); |
| QVSNST | (SEQ ID NO: 595); |
| QVSQAQ | (SEQ ID NO: 596); |
| QVSQTG | (SEQ ID NO: 597); |
| QVSQTQ | (SEQ ID NO: 598); |
| QVSSAQ | (SEQ ID NO: 599); |
| QVSSSG | (SEQ ID NO: 600); |
| QVSSSQ | (SEQ ID NO: 601); |
| QVSSSS | (SEQ ID NO: 602); |
| QVSSTQ | (SEQ ID NO: 603); |
| QVSTSG | (SEQ ID NO: 604); |
| QVSTSN | (SEQ ID NO: 605); |
| QVSTSS | (SEQ ID NO: 606); |
| QVSTST | (SEQ ID NO: 607); |
| SAQQSQ | (SEQ ID NO: 608); |
| SAQQTQ | (SEQ ID NO: 609); |
| SASQQQ | (SEQ ID NO: 610); |
| SASQSQ | (SEQ ID NO: 611); |
| SGNSTV | (SEQ ID NO: 612); |
| SGNTSV | (SEQ ID NO: 613); |
| SGNVST | (SEQ ID NO: 614); |
| SGNVTS | (SEQ ID NO: 615); |
| SGQQTQ | (SEQ ID NO: 616); |
| SGQQTV | (SEQ ID NO: 617); |
| SGQTSV | (SEQ ID NO: 618); |
| SGQVSS | (SEQ ID NO: 619); |
| SGQVTG | (SEQ ID NO: 620); |
| SGQVTQ | (SEQ ID NO: 621); |
| SGQVTT | (SEQ ID NO: 622); |
| SGSNTV | (SEQ ID NO: 623); |
| SGSTNV | (SEQ ID NO: 624); |
| SGSTQV | (SEQ ID NO: 625); |
| SGSVAS | (SEQ ID NO: 626); |
| SGSVNT | (SEQ ID NO: 627); |
| SGSVQG | (SEQ ID NO: 628); |
| SGSVQS | (SEQ ID NO: 629); |
| SNNQTV | (SEQ ID NO: 630); |
| SNNTAV | (SEQ ID NO: 631); |
| SNNTNV | (SEQ ID NO: 632); |
| SNNTQV | (SEQ ID NO: 633); |
| SNQQSQ | (SEQ ID NO: 634); |
| SNQQSV | (SEQ ID NO: 635); |
| SNQQTQ | (SEQ ID NO: 636); |
| SNQQTV | (SEQ ID NO: 637); |
| SNQSTV | (SEQ ID NO: 638); |
| SNQTSV | (SEQ ID NO: 639); |
| SNQVSG | (SEQ ID NO: 640); |
| SNQVSQ | (SEQ ID NO: 641); |
| SNQVSS | (SEQ ID NO: 642); |
| SNQVST | (SEQ ID NO: 643); |
| SNQVTG | (SEQ ID NO: 644); |
| SNQVTQ | (SEQ ID NO: 645); |
| SNQVTS | (SEQ ID NO: 646); |
| SNSGAV | (SEQ ID NO: 647); |
| SNSNAV | (SEQ ID NO: 648); |
| SNSNGV | (SEQ ID NO: 649); |
| SNSNQS | (SEQ ID NO: 650); |
| SNSQAG | (SEQ ID NO: 651); |
| SNSQAQ | (SEQ ID NO: 652); |
| SNSQAS | (SEQ ID NO: 653); |
| SNSQAT | (SEQ ID NO: 654); |
| SNSQAV | (SEQ ID NO: 655); |
| SNSQGQ | (SEQ ID NO: 656); |
| SNSQGV | (SEQ ID NO: 657); |
| SNSQQA | (SEQ ID NO: 658); |
| SNSQQG | (SEQ ID NO: 659); |
| SNSQQQ | (SEQ ID NO: 660); |
| SNSQQS | (SEQ ID NO: 661); |
| SNSQQT | (SEQ ID NO: 662); |
| SNSQQV | (SEQ ID NO: 663); |
| SNSQSG | (SEQ ID NO: 664); |
| SNSQST | (SEQ ID NO: 665); |
| SNSQSV | (SEQ ID NO: 666); |
| SNSSAV | (SEQ ID NO: 667); |
| SNSSGV | (SEQ ID NO: 668); |
| SNSSQG | (SEQ ID NO: 669); |
| SNSSQQ | (SEQ ID NO: 670); |
| SNSSQT | (SEQ ID NO: 671); |
| SNSSQV | (SEQ ID NO: 672); |
| SNSSSQ | (SEQ ID NO: 673); |
| SNSSSV | (SEQ ID NO: 674); |
| SNSTAG | (SEQ ID NO: 675); |
| SNSTGS | (SEQ ID NO: 676); |
| SNSTNV | (SEQ ID NO: 677); |
| SNSTQA | (SEQ ID NO: 678); |
| SNSTQQ | (SEQ ID NO: 679); |
| SNSTQS | (SEQ ID NO: 680); |
| SNSTQV | (SEQ ID NO: 681); |
| SNSTSA | (SEQ ID NO: 682); |
| SNSTSV | (SEQ ID NO: 683); |
| SNSTVG | (SEQ ID NO: 684); |
| SNSVAQ | (SEQ ID NO: 685); |
| SNSVAS | (SEQ ID NO: 686); |
| SNSVAT | (SEQ ID NO: 687); |
| SNSVGQ | (SEQ ID NO: 688); |
| SNSVGS | (SEQ ID NO: 689); |
| SNSVGT | (SEQ ID NO: 690); |
| SNSVNS | (SEQ ID NO: 691); |
| SNSVNT | (SEQ ID NO: 692); |
| SNSVQQ | (SEQ ID NO: 693); |
| SNSVQS | (SEQ ID NO: 694); |
| SNSVSG | (SEQ ID NO: 695); |
| SNSVSQ | (SEQ ID NO: 696); |
| SNSVSS | (SEQ ID NO: 697); |
| SNSVST | (SEQ ID NO: 698); |
| SNSVTG | (SEQ ID NO: 699); |
| SQNVAS | (SEQ ID NO: 700); |
| SQNVNS | (SEQ ID NO: 701); |
| SQQQSQ | (SEQ ID NO: 702); |
| SQQQSV | (SEQ ID NO: 703); |
| SQQQTQ | (SEQ ID NO: 704); |
| SQQQTV | (SEQ ID NO: 705); |
| SQQSTV | (SEQ ID NO: 706); |
| SQQVSG | (SEQ ID NO: 707); |
| SQQVSN | (SEQ ID NO: 708); |
| SQQVSS | (SEQ ID NO: 709); |
| SQQVST | (SEQ ID NO: 710); |
| SQQVTG | (SEQ ID NO: 711); |
| SQQVTN | (SEQ ID NO: 712); |
| SQQVTS | (SEQ ID NO: 713); |
| SQQVTT | (SEQ ID NO: 714); |
| SQSNAS | (SEQ ID NO: 715); |
| SQSNAV | (SEQ ID NO: 716); |
| SQSNGV | (SEQ ID NO: 717); |
| SQSNQT | (SEQ ID NO: 718); |
| SQSNQV | (SEQ ID NO: 719); |
| SQSNSV | (SEQ ID NO: 720); |
| SQSQAQ | (SEQ ID NO: 721); |
| SQSQAS | (SEQ ID NO: 722); |
| SQSQAV | (SEQ ID NO: 723); |
| SQSQQQ | (SEQ ID NO: 724); |
| SQSQQV | (SEQ ID NO: 725); |
| SQSQSQ | (SEQ ID NO: 726); |
| SQSSAV | (SEQ ID NO: 727); |
| SQSSGV | (SEQ ID NO: 728); |
| SQSSQG | (SEQ ID NO: 729); |
| SQSSQV | (SEQ ID NO: 730); |
| SQSSSQ | (SEQ ID NO: 731); |
| SQSSSV | (SEQ ID NO: 732); |
| SQSTAV | (SEQ ID NO: 733); |
| SQSTQS | (SEQ ID NO: 734); |
| SQSTSG | (SEQ ID NO: 735); |
| SQSTSV | (SEQ ID NO: 736); |
| SQSVAG | (SEQ ID NO: 737); |
| SQSVAN | (SEQ ID NO: 738); |
| SQSVAQ | (SEQ ID NO: 739); |
| SQSVAS | (SEQ ID NO: 740); |
| SQSVAT | (SEQ ID NO: 741); |
| SQSVGG | (SEQ ID NO: 742); |
| SQSVGN | (SEQ ID NO: 743); |
| SQSVGQ | (SEQ ID NO: 744); |
| SQSVGS | (SEQ ID NO: 745); |
| SQSVGT | (SEQ ID NO: 746); |
| SQSVNG | (SEQ ID NO: 747); |
| SQSVNS | (SEQ ID NO: 748); |
| SQSVQG | (SEQ ID NO: 749); |
| SQSVQN | (SEQ ID NO: 750); |
| SQSVQQ | (SEQ ID NO: 751); |
| SQSVQS | (SEQ ID NO: 752); |
| SQSVQT | (SEQ ID NO: 753); |
| SQSVSG | (SEQ ID NO: 754); |
| SQSVSN | (SEQ ID NO: 755); |
| SQSVSS | (SEQ ID NO: 756); |
| SQSVST | (SEQ ID NO: 757); |
| SSNGTV | (SEQ ID NO: 758); |
| SSNQNQ | (SEQ ID NO: 759); |
| SSNQNV | (SEQ ID NO: 760); |
| SSNQTQ | (SEQ ID NO: 761); |
| SSNQTV | (SEQ ID NO: 762); |
| SSNSTV | (SEQ ID NO: 763); |
| SSNTNV | (SEQ ID NO: 764); |
| SSNTQV | (SEQ ID NO: 765); |
| SSNTVG | (SEQ ID NO: 766); |
| SSNVAQ | (SEQ ID NO: 767); |
| SSNVAS | (SEQ ID NO: 768); |
| SSNVGT | (SEQ ID NO: 769); |
| SSNVNG | (SEQ ID NO: 770); |
| SSNVNQ | (SEQ ID NO: 771); |
| SSNVNS | (SEQ ID NO: 772); |
| SSNVNT | (SEQ ID NO: 773); |
| SSNVQS | (SEQ ID NO: 774); |
| SSNVQT | (SEQ ID NO: 775); |
| SSNVTG | (SEQ ID NO: 776); |
| SSNVTQ | (SEQ ID NO: 777); |
| SSNVTS | (SEQ ID NO: 778); |
| SSQNTV | (SEQ ID NO: 779); |
| SSQQSV | (SEQ ID NO: 780); |
| SSQQTQ | (SEQ ID NO: 781); |
| SSQQTV | (SEQ ID NO: 782); |
| SSQSTQ | (SEQ ID NO: 783); |
| SSQTSV | (SEQ ID NO: 784); |
| SSQVNS | (SEQ ID NO: 785); |
| SSQVSN | (SEQ ID NO: 786); |
| SSQVSQ | (SEQ ID NO: 787); |
| SSQVSS | (SEQ ID NO: 788); |
| SSQVST | (SEQ ID NO: 789); |
| SSQVTG | (SEQ ID NO: 790); |
| SSQVTQ | (SEQ ID NO: 791); |
| SSQVTS | (SEQ ID NO: 792); |
| SSQVTT | (SEQ ID NO: 793); |
| SSSNAV | (SEQ ID NO: 794); |
| SSSNQV | (SEQ ID NO: 795); |
| SSSNSV | (SEQ ID NO: 796); |
| SSSQAQ | (SEQ ID NO: 797); |
| SSSQNQ | (SEQ ID NO: 798); |
| SSSQQQ | (SEQ ID NO: 799); |
| SSSQQT | (SEQ ID NO: 800); |
| SSSQQV | (SEQ ID NO: 801); |
| SSSQSQ | (SEQ ID NO: 802); |
| SSSQSV | (SEQ ID NO: 803); |
| SSSSGQ | (SEQ ID NO: 804); |
| SSSSGV | (SEQ ID NO: 805); |
| SSSSNV | (SEQ ID NO: 806); |
| SSSSQQ | (SEQ ID NO: 807); |
| SSSSQS | (SEQ ID NO: 808); |
| SSSTAV | (SEQ ID NO: 809); |
| SSSTNV | (SEQ ID NO: 810); |
| SSSTSV | (SEQ ID NO: 811); |
| SSSVAQ | (SEQ ID NO: 812); |
| SSSVAS | (SEQ ID NO: 813); |
| SSSVGG | (SEQ ID NO: 814); |
| SSSVGQ | (SEQ ID NO: 815); |
| SSSVGS | (SEQ ID NO: 816); |
| SSSVNG | (SEQ ID NO: 817); |
| SSSVNS | (SEQ ID NO: 818); |
| SSSVNT | (SEQ ID NO: 819); |
| SSSVQG | (SEQ ID NO: 820); |
| SSSVQN | (SEQ ID NO: 821); |
| SSSVQS | (SEQ ID NO: 822); |
| SSSVQT | (SEQ ID NO: 823); |
| SSSVSG | (SEQ ID NO: 824); |
| SSSVSN | (SEQ ID NO: 825); |
| SSSVSQ | (SEQ ID NO: 826); |
| SSSVSS | (SEQ ID NO: 827); |
| SSSVST | (SEQ ID NO: 828); |
| STNGSV | (SEQ ID NO: 829); |
| STNSGV | (SEQ ID NO: 830); |
| STNSNV | (SEQ ID NO: 831); |
| STNTNV | (SEQ ID NO: 832); |
| STNTQV | (SEQ ID NO: 833); |
| STNVGS | (SEQ ID NO: 834); |
| STNVNG | (SEQ ID NO: 835); |
| STNVNS | (SEQ ID NO: 836); |
| STNVSG | (SEQ ID NO: 837); |
| STNVSS | (SEQ ID NO: 838); |
| STNVTG | (SEQ ID NO: 839); |
| STNVTS | (SEQ ID NO: 840); |
| STQQSG | (SEQ ID NO: 841); |
| STQQSQ | (SEQ ID NO: 842); |
| STQQSV | (SEQ ID NO: 843); |
| STQQTQ | (SEQ ID NO: 844); |
| STQQTS | (SEQ ID NO: 845); |
| STQQTV | (SEQ ID NO: 846); |
| STQSNV | (SEQ ID NO: 847); |
| STQSTV | (SEQ ID NO: 848); |
| STQTSS | (SEQ ID NO: 849); |
| STQTSV | (SEQ ID NO: 850); |
| STQVNS | (SEQ ID NO: 851); |
| STQVSG | (SEQ ID NO: 852); |
| STQVSN | (SEQ ID NO: 853); |
| STQVST | (SEQ ID NO: 854); |
| STQVTG | (SEQ ID NO: 855); |
| STQVTN | (SEQ ID NO: 856); |
| STQVTQ | (SEQ ID NO: 857); |
| STQVTS | (SEQ ID NO: 858); |
| STQVTT | (SEQ ID NO: 859); |
| STSGNV | (SEQ ID NO: 860); |
| STSNAS | (SEQ ID NO: 861); |
| STSNGV | (SEQ ID NO: 862); |
| STSNQV | (SEQ ID NO: 863); |
| STSNSV | (SEQ ID NO: 864); |
| STSQAQ | (SEQ ID NO: 865); |
| STSQGV | (SEQ ID NO: 866); |
| STSQNQ | (SEQ ID NO: 867); |
| STSQQQ | (SEQ ID NO: 868); |
| STSQQV | (SEQ ID NO: 869); |
| STSQSQ | (SEQ ID NO: 870); |
| STSSAV | (SEQ ID NO: 871); |
| STSSGV | (SEQ ID NO: 872); |
| STSSQQ | (SEQ ID NO: 873); |
| STSSQS | (SEQ ID NO: 874); |
| STSSQV | (SEQ ID NO: 875); |
| STSSSQ | (SEQ ID NO: 876); |
| STSTAV | (SEQ ID NO: 877); |
| STSTGV | (SEQ ID NO: 878); |
| STSTQA | (SEQ ID NO: 879); |
| STSTQG | (SEQ ID NO: 880); |
| STSTQT | (SEQ ID NO: 881); |
| STSTQV | (SEQ ID NO: 882); |
| STSTSQ | (SEQ ID NO: 883); |
| STSTSV | (SEQ ID NO: 884); |
| STSVAG | (SEQ ID NO: 885); |
| STSVAN | (SEQ ID NO: 886); |
| STSVAQ | (SEQ ID NO: 887); |
| STSVAS | (SEQ ID NO: 888); |
| STSVAT | (SEQ ID NO: 889); |
| STSVGG | (SEQ ID NO: 890); |
| STSVGN | (SEQ ID NO: 891); |
| STSVGQ | (SEQ ID NO: 892); |
| STSVGS | (SEQ ID NO: 893); |
| STSVNG | (SEQ ID NO: 894); |
| STSVNN | (SEQ ID NO: 895); |
| STSVNS | (SEQ ID NO: 896); |
| STSVQG | (SEQ ID NO: 897); |
| STSVQN | (SEQ ID NO: 898); |
| STSVQQ | (SEQ ID NO: 899); |
| STSVQS | (SEQ ID NO: 900); |
| STSVQT | (SEQ ID NO: 901); |
| STSVSG | (SEQ ID NO: 902); |
| STSVSN | (SEQ ID NO: 903); |
| STSVSQ | (SEQ ID NO: 904); |
| STSVSS | (SEQ ID NO: 905); |
| STSVST | (SEQ ID NO: 906); |
| SVNGST | (SEQ ID NO: 907); |
| SVNGTS | (SEQ ID NO: 908); |
| SVNQAQ | (SEQ ID NO: 909); |
| SVNQQQ | (SEQ ID NO: 910); |
| SVNSGT | (SEQ ID NO: 911); |
| SVNSNQ | (SEQ ID NO: 912); |
| SVNSNS | (SEQ ID NO: 913); |
| SVNSTG | (SEQ ID NO: 914); |
| SVNSTS | (SEQ ID NO: 915); |
| SVNTGS | (SEQ ID NO: 916); |
| SVNTSG | (SEQ ID NO: 917); |
| SVQQSQ | (SEQ ID NO: 918); |
| SVQQST | (SEQ ID NO: 919); |
| SVQQTQ | (SEQ ID NO: 920); |
| SVQQTS | (SEQ ID NO: 921); |
| SVQQTT | (SEQ ID NO: 922); |
| SVQSNQ | (SEQ ID NO: 923); |
| SVQSNS | (SEQ ID NO: 924); |
| SVQSSG | (SEQ ID NO: 925); |
| SVQSSQ | (SEQ ID NO: 926); |
| SVQSSS | (SEQ ID NO: 927); |
| SVQSTG | (SEQ ID NO: 928); |
| SVQSTQ | (SEQ ID NO: 929); |
| SVQSTS | (SEQ ID NO: 930); |
| SVQTSG | (SEQ ID NO: 931); |
| SVQTSN | (SEQ ID NO: 932); |
| SVQTSS | (SEQ ID NO: 933); |
| SVQVSN | (SEQ ID NO: 934); |
| SVSGNT | (SEQ ID NO: 935); |
| SVSGQS | (SEQ ID NO: 936); |
| SVSNAQ | (SEQ ID NO: 937); |
| SVSNAS | (SEQ ID NO: 938); |
| SVSNGS | (SEQ ID NO: 939); |
| SVSNGT | (SEQ ID NO: 940); |
| SVSNQG | (SEQ ID NO: 941); |
| SVSNQS | (SEQ ID NO: 942); |
| SVSNQT | (SEQ ID NO: 943); |
| SVSNST | (SEQ ID NO: 944); |
| SVSNTG | (SEQ ID NO: 945); |
| SVSQAQ | (SEQ ID NO: 946); |
| SVSQAS | (SEQ ID NO: 947); |
| SVSQGQ | (SEQ ID NO: 948); |
| SVSQNQ | (SEQ ID NO: 949); |
| SVSQQG | (SEQ ID NO: 950); |
| SVSQQQ | (SEQ ID NO: 951); |
| SVSQQS | (SEQ ID NO: 952); |
| SVSQSG | (SEQ ID NO: 953); |
| SVSQSS | (SEQ ID NO: 954); |
| SVSQTQ | (SEQ ID NO: 955); |
| SVSSAQ | (SEQ ID NO: 956); |
| SVSSAS | (SEQ ID NO: 957); |
| SVSSGG | (SEQ ID NO: 958); |
| SVSSGS | (SEQ ID NO: 959); |
| SVSSGT | (SEQ ID NO: 960); |
| SVSSNG | (SEQ ID NO: 961); |
| SVSSNQ | (SEQ ID NO: 962); |
| SVSSNS | (SEQ ID NO: 963); |
| SVSSNT | (SEQ ID NO: 964); |
| SVSSQG | (SEQ ID NO: 965); |
| SVSSQQ | (SEQ ID NO: 966); |
| SVSSQS | (SEQ ID NO: 967); |
| SVSSQT | (SEQ ID NO: 968); |
| SVSSQV | (SEQ ID NO: 969); |
| SVSSSG | (SEQ ID NO: 970); |
| SVSSSQ | (SEQ ID NO: 971); |
| SVSSSS | (SEQ ID NO: 972); |
| SVSTAG | (SEQ ID NO: 973); |
| SVSTAS | (SEQ ID NO: 974); |
| SVSTAT | (SEQ ID NO: 975); |
| SVSTGS | (SEQ ID NO: 976); |
| SVSTNS | (SEQ ID NO: 977); |
| SVSTNT | (SEQ ID NO: 978); |
| SVSTQQ | (SEQ ID NO: 979); |
| SVSTQS | (SEQ ID NO: 980); |
| SVSTQT | (SEQ ID NO: 981); |
| SVSTSG | (SEQ ID NO: 982); |
| SVSTSN | (SEQ ID NO: 983); |
| SVSTSS | (SEQ ID NO: 984); |
| SVSTST | (SEQ ID NO: 985); |
| TASQSQ | (SEQ ID NO: 986); |
| TASQVQ | (SEQ ID NO: 987); |
| TGSNSV | (SEQ ID NO: 988); |
| TGSNVS | (SEQ ID NO: 989); |
| TGSSNV | (SEQ ID NO: 990); |
| TGSVNS | (SEQ ID NO: 991); |
| TGSVSN | (SEQ ID NO: 992); |
| TNSGSV | (SEQ ID NO: 993); |
| TNSGVS | (SEQ ID NO: 994); |
| TNSQVQ | (SEQ ID NO: 995); |
| TNSSGV | (SEQ ID NO: 996); |
| TNSVGS | (SEQ ID NO: 997); |
| TNSVSG | (SEQ ID NO: 998); |
| TQSQSQ | (SEQ ID NO: 999); |
| TQSQVQ | (SEQ ID NO: 1000); |
| TQSTVS | (SEQ ID NO: 1001); |
| TQSVSN | (SEQ ID NO: 1002); |
| TQSVSS | (SEQ ID NO: 1003); |
| TSNGVS | (SEQ ID NO: 1004); |
| TSNVGS | (SEQ ID NO: 1005); |
| TSNVSG | (SEQ ID NO: 1006); |
| TSSGNV | (SEQ ID NO: 1007); |
| TSSGVN | (SEQ ID NO: 1008); |
| TSSNGV | (SEQ ID NO: 1009); |
| TSSNSV | (SEQ ID NO: 1010); |
| TSSNVG | (SEQ ID NO: 1011); |
| TSSQSQ | (SEQ ID NO: 1012); |
| TSSSVQ | (SEQ ID NO: 1013); |
| TSSVGN | (SEQ ID NO: 1014); |
| TSSVNG | (SEQ ID NO: 1015); |
| TSSVSG | (SEQ ID NO: 1016); |
| TSSVSS | (SEQ ID NO: 1017); |
| TSSVTS | (SEQ ID NO: 1018); |
| TVNSGS | (SEQ ID NO: 1019); |
| TVNSSG | (SEQ ID NO: 1020); |
| TVSGNS | (SEQ ID NO: 1021); |
| TVSGSN | (SEQ ID NO: 1022); |
| TVSNGS | (SEQ ID NO: 1023); |
| TVSNSG | (SEQ ID NO: 1024); |
| TVSNST | (SEQ ID NO: 1025); |
| TVSNVG | (SEQ ID NO: 1026); |
| TVSQNQ | (SEQ ID NO: 1027); |
| TVSQQV | (SEQ ID NO: 1028); |
| TVSQSG | (SEQ ID NO: 1029); |
| TVSQSQ | (SEQ ID NO: 1030); |
| TVSSGN | (SEQ ID NO: 1031); |
| TVSSNG | (SEQ ID NO: 1032); |
| TVSSNQ | (SEQ ID NO: 1033); |
| TVSSNS | (SEQ ID NO: 1034); |
| TVSSSQ | (SEQ ID NO: 1035); |
| TVSTSG | (SEQ ID NO: 1036); |
| TVSTSN | (SEQ ID NO: 1037); |
| TVSTSS | (SEQ ID NO: 1038); |
| TVSTST | (SEQ ID NO: 1039); |
| VGSTNS | (SEQ ID NO: 1040); |
| VNSTSG | (SEQ ID NO: 1041); |
| VNSTSN | (SEQ ID NO: 1042); |
| VSSQSQ | (SEQ ID NO: 1043); |
| VSSQVQ | (SEQ ID NO: 1044); |
| VSSTNG | (SEQ ID NO: 1045); |
| VTSNSG | (SEQ ID NO: 1046); |
| and | |
| VTSQSQ | (SEQ ID NO: 1047). |

3. The composition according to claim 2, wherein the hexapeptide comprises 1, 2 or 3 amino acids having polar basic side chains.

4. The composition according to claim 2, wherein the hexapeptide comprises 1 amino acid having a polar acidic side chain.

5. The composition according to claim 2, wherein the hexapeptide comprises 0, 1, 2, 3, 4, or 5 amino acids having hydrophobic side chains.

6. The composition according to claim 2, wherein the hexapeptide comprises 0, 1, 2, 3, 4, or 5 amino acids having polar uncharged side chains.

7. A composition for treatment of one or more inflammatory conditions in a mammal wherein the composition comprises a fibril, and wherein the fibril comprises four or more hexapeptides comprising amino acids of an L-configuration, D-configuration or a mixture of configurations having free energy of formation of −23 kcal/mol or less when the hexapeptide sequence in subjected to the Rosetta-Profile algorithm.

8. The fibril according to claim 7, wherein the hexapeptides are selected from a group consisting of:

| | |
|---|---|
| SVNVDL | (SEQ ID NO: 1); |
| SLNVDV | (SEQ ID NO: 2); |
| SVDVNL | (SEQ ID NO: 3); |
| DLSVVL | (SEQ ID NO: 4), |
| SVNLDV | (SEQ ID NO: 5), |
| SVVNDV | (SEQ ID NO: 6), |
| DVSLVN | (SEQ ID NO: 7); |
| DVSVLN | (SEQ ID NO: 8); |
| SDLVNV | (SEQ ID NO: 9); |
| SLNVVS | (SEQ ID NO: 10); |
| LNVDSV | (SEQ ID NO: 11); |
| NDLSVV | (SEQ ID NO: 12); |
| VDNLVS | (SEQ ID NO: 13); |
| VNDVSL | (SEQ ID NO: 14); |
| VSDNVL | (SEQ ID NO: 15); |
| LNDVVS | (SEQ ID NO: 16); |
| LSVDVN | (SEQ ID NO: 17); |
| NSVDLV | (SEQ ID NO: 18); |
| VDVLNS | (SEQ ID NO: 19); |
| VNDSVL | (SEQ ID NO: 20); |
| VNVSLD | (SEQ ID NO: 21); |
| LDVNSV | (SEQ ID NO: 22); |
| LNVVDS | (SEQ ID NO: 23); |
| LSDVVN | (SEQ ID NO: 24); |
| LVVNDS | (SEQ ID NO: 25); |
| NSLDVV | (SEQ ID NO: 26); |
| VLVDNS | (SEQ ID NO: 27); |
| VNLSDV | (SEQ ID NO: 28); |
| VNVDLS | (SEQ ID NO: 29); |
| DNVSVD | (SEQ ID NO: 30); |
| LSDNVV | (SEQ ID NO: 31); |
| LSVVDN | (SEQ ID NO: 32); |
| VLDVSN | (SEQ ID NO: 33); |
| VNDLVS | (SEQ ID NO: 34); |
| VNVSDL | (SEQ ID NO: 35); |
| VVLSDN | (SEQ ID NO: 36); |
| LVSVNL | (SEQ ID NO: 37); |
| LVNVSV | (SEQ ID NO: 38); |
| SLNVSV | (SEQ ID NO: 39); |
| LVSVNS | (SEQ ID NO: 40); |
| SVDVNV | (SEQ ID NO: 41); |
| LVVSVL | (SEQ ID NO: 42); |
| VNLVVS | (SEQ ID NO: 43); |
| SNLVSV | (SEQ ID NO: 44); |
| SVNVLS | (SEQ ID NO: 45); |
| VLVSVL | (SEQ ID NO: 46); |
| LVNVSL | (SEQ ID NO: 47); |
| SVNVDS | (SEQ ID NO: 48); |
| VLSVNV | (SEQ ID NO: 49); |
| NLVVSV | (SEQ ID NO: 50); |
| SVVLNV | (SEQ ID NO: 51); |
| LSVVNL | (SEQ ID NO: 52); |
| SNLVVS | (SEQ ID NO: 53); |
| SVVLDV | (SEQ ID NO: 54); |
| LVSLNV | (SEQ ID NO: 55); |
| VSLNVV | (SEQ ID NO: 56); |
| VKVQIY | (SEQ ID NO: 57); |
| QVVIYK | (SEQ ID NO: 58); |
| KVIQVY | (SEQ ID NO: 59); |
| VYVKIY | (SEQ ID NO: 60); |
| QIVVYK | (SEQ ID NO: 61); |
| QVIKVY | (SEQ ID NO: 62); |
| VQVKIY | (SEQ ID NO: 63); |
| QVVKIY | (SEQ ID NO: 64); |
| QVIVYK | (SEQ ID NO: 65); |
| QIVKVY | (SEQ ID NO: 66); |
| QKIVVY | (SEQ ID NO: 67); |
| QKVVYI | (SEQ ID NO: 68); |
| KVQVYI | (SEQ ID NO: 69); |
| QVVKYI | (SEQ ID NO: 70); |
| KVQIYV | (SEQ ID NO: 71); |
| VKIQVY | (SEQ ID NO: 72); |
| VIQKVY | (SEQ ID NO: 73); |
| KVVIYK | (SEQ ID NO: 74); |
| QIVKYV | (SEQ ID NO: 75); |
| QKVIYV | (SEQ ID NO: 76); |
| KQVVIY | (SEQ ID NO: 77); |
| KIQVYV | (SEQ ID NO: 78); |
| KVYVQI | (SEQ ID NO: 79); |
| VVQIYK | (SEQ ID NO: 80); |
| KQVIVY | (SEQ ID NO: 81); |
| VQIKVY | (SEQ ID NO: 82); |
| QKIVYV | (SEQ ID NO: 83); |
| VIQVYK | (SEQ ID NO: 84); |
| KVVIQY | (SEQ ID NO: 85); |
| KVQVIY | (SEQ ID NO: 86); |
| QYVVIK | (SEQ ID NO: 87); |
| YVVIQK | (SEQ ID NO: 88); |
| KIVVQY | (SEQ ID NO: 89); |
| YQVIVK | (SEQ ID NO: 90); |
| KIYVQV | (SEQ ID NO: 91); |
| KVVIYQ | (SEQ ID NO: 92); |
| KYVVQI | (SEQ ID NO: 93); |
| YQIVVK | (SEQ ID NO: 94); |
| YVVIQY | (SEQ ID NO: 95); |
| KQVIYV | (SEQ ID NO: 96); |
| KYVQVI | (SEQ ID NO: 97); |
| KYVVIQ | (SEQ ID NO: 98); |
| QKVVIY | (SEQ ID NO: 99); |
| QYVIVK | (SEQ ID NO: 100); |
| IVQKVY | (SEQ ID NO: 101); |
| KYIVVQ | (SEQ ID NO: 102); |
| QVIKYV | (SEQ ID NO: 103); |
| YIVVQK | (SEQ ID NO: 104); |
| YQVVIK | (SEQ ID NO: 105); |
| IKVQVY | (SEQ ID NO: 106); |
| KVVQYI | (SEQ ID NO: 107); |
| KYVQIV | (SEQ ID NO: 108); |
| VKQVYI | (SEQ ID NO: 109); |
| VQVIYK | (SEQ ID NO: 110); |
| VVIQKY | (SEQ ID NO: 111); |
| IQVVYK | (SEQ ID NO: 112); |
| KIVVYQ | (SEQ ID NO: 113); |
| KYQVIV | (SEQ ID NO: 114); |
| KYVIVQ | (SEQ ID NO: 115); |
| VVYIQK | (SEQ ID NO: 116); |
| IVQVYK | (SEQ ID NO: 117); |
| KQIVYV | (SEQ ID NO: 118); |
| KYVIQV | (SEQ ID NO: 119); |
| QKVIVY | (SEQ ID NO: 120); |
| QYIKVV | (SEQ ID NO: 121); |
| QYIVKV | (SEQ ID NO: 122); |
| VQIVYK | (SEQ ID NO: 123); |
| VVQKYI | (SEQ ID NO: 124); |
| VYQIVK | (SEQ ID NO: 125); |
| YVQVIK | (SEQ ID NO: 126); |
| QIVYVK | (SEQ ID NO: 127); |
| IKVYQV | (SEQ ID NO: 128); |
| KVVYIQ | (SEQ ID NO: 129); |
| VQKYIV | (SEQ ID NO: 130); |
| VQKYVI | (SEQ ID NO: 131); |
| KVVQIY | (SEQ ID NO: 132); |
| YVIKIY | (SEQ ID NO: 133); |
| QVIVYV | (SEQ ID NO: 134); |
| VVIKVY | (SEQ ID NO: 135); |
| KVVIYI | (SEQ ID NO: 136); |
| VYVVIK | (SEQ ID NO: 137); |
| YIVQVY | (SEQ ID NO: 138); |
| YVIQVY | (SEQ ID NO: 139); |
| YVIVVY | (SEQ ID NO: 140); |
| KVIVYI | (SEQ ID NO: 141); |
| YVIKVY | (SEQ ID NO: 142); |
| VQVIVK | (SEQ ID NO: 143); |
| YVVKIY | (SEQ ID NO: 144); |
| YVVQVI | (SEQ ID NO: 145); |
| QIVVYQ | (SEQ ID NO: 146); |
| VVQIVK | (SEQ ID NO: 147); |
| VQIVVK | (SEQ ID NO: 148); |
| VIVVYK | (SEQ ID NO: 149); |
| IVQVYI | (SEQ ID NO: 150); |
| YVVIQV | (SEQ ID NO: 151); |
| VYQVVI | (SEQ ID NO: 152); |
| KYIQVY | (SEQ ID NO: 153); |
| KYVVIY | (SEQ ID NO: 154); |
| KYVQIY | (SEQ ID NO: 155); |
| VVQVIK | (SEQ ID NO: 156); |
| QKIVVK | (SEQ ID NO: 157); |
| KYVIVY | (SEQ ID NO: 158); |
| KQVVIK | (SEQ ID NO: 159); |
| YVQIYV | (SEQ ID NO: 160); |
| YKVQVY | (SEQ ID NO: 161); |
| IKVQIY | (SEQ ID NO: 162); |
| KIVVQK | (SEQ ID NO: 163); |
| VIKVVI | (SEQ ID NO: 164); |
| VYIKVV | (SEQ ID NO: 165); |
| VYIVQV | (SEQ ID NO: 166); |
| IVYVQI | (SEQ ID NO: 167); |
| KVQIYK | (SEQ ID NO: 168); |
| IYVIVY | (SEQ ID NO: 169); |
| VIYVIV | (SEQ ID NO: 170); |
| KYQVYI | (SEQ ID NO: 171); |
| VIQKVV | (SEQ ID NO: 172); |
| VKIVYV | (SEQ ID NO: 173); |
| VKQIVV | (SEQ ID NO: 174); |
| YIVKQY | (SEQ ID NO: 175); |
| YIVVQY | (SEQ ID NO: 176); |
| YKIQVY | (SEQ ID NO: 177); |
| YQVVIY | (SEQ ID NO: 178); |
| YVVKQY | (SEQ ID NO: 179); |
| VIVKVQ | (SEQ ID NO: 180); |
| QKVVIK | (SEQ ID NO: 181); |
| GMVVVG | (SEQ ID NO: 182); |
| GVVVMG | (SEQ ID NO: 183); |
| GVVMVG | (SEQ ID NO: 184); |
| GGVVVM | (SEQ ID NO: 185); |
| GVVGVM | (SEQ ID NO: 186); |
| GVVVGM | (SEQ ID NO: 187); |
| GVMVVG | (SEQ ID NO: 188); |
| GGVVMV | (SEQ ID NO: 189); |
| GGVMVV | (SEQ ID NO: 190); |
| GMVVGV | (SEQ ID NO: 191); |
| GVVMGV | (SEQ ID NO: 192); |
| GMVGVV | (SEQ ID NO: 193); |
| GVMVGV | (SEQ ID NO: 194); |
| GVVGMV | (SEQ ID NO: 195); |
| GGMVVV | (SEQ ID NO: 196); |
| GVGVMV | (SEQ ID NO: 197); |
| GVGVVM | (SEQ ID NO: 198); |
| MVVVGM | (SEQ ID NO: 199); |
| GMGVVV | (SEQ ID NO: 200); |
| MVVVGG | (SEQ ID NO: 201); |
| GVGMVV | (SEQ ID NO: 202); |
| GVVGGV | (SEQ ID NO: 203); |
| MVGVVG | (SEQ ID NO: 204); |
| VVMVGG | (SEQ ID NO: 205); |
| MGVVVG | (SEQ ID NO: 206); |
| MVGVGV | (SEQ ID NO: 207); |
| VGVMVG | (SEQ ID NO: 208); |
| MVVGVG | (SEQ ID NO: 209); |
| VVGVMG | (SEQ ID NO: 210); |
| VMGVVG | (SEQ ID NO: 211); |
| VGVGVM | (SEQ ID NO: 212); |
| VMVGVG | (SEQ ID NO: 213); |
| MGGVVV | (SEQ ID NO: 214); |
| VGMVVG | (SEQ ID NO: 215); |
| VVGVGM | (SEQ ID NO: 216); |
| VGGVVM | (SEQ ID NO: 217); |
| VVGMVG | (SEQ ID NO: 218); |
| VGVVMG | (SEQ ID NO: 219); |
| MVGGVV | (SEQ ID NO: 220); |
| VGMVGV | (SEQ ID NO: 221); |
| VMVVGG | (SEQ ID NO: 222); |
| VVGGVM | (SEQ ID NO: 223); |
| VGGVMV | (SEQ ID NO: 224); |
| VVVGMG | (SEQ ID NO: 225); |
| VVGMGV | (SEQ ID NO: 226); |
| MGVGVV | (SEQ ID NO: 227); |
| VGVVGM | (SEQ ID NO: 228); |
| VGGMVV | (SEQ ID NO: 229); |
| VVVMGG | (SEQ ID NO: 230); |
| VVMGGV | (SEQ ID NO: 231); |
| VVVGGM | (SEQ ID NO: 232); |
| VVGGMV | (SEQ ID NO: 233); |
| VGMGVV | (SEQ ID NO: 234); |
| VMGGVV | (SEQ ID NO: 235); |
| VGGVGV | (SEQ ID NO: 236); |
| VGVMGV | (SEQ ID NO: 237); |
| VGVGMV | (SEQ ID NO: 238); |
| and | |
| VMVGGV | (SEQ ID NO: 239); |
| ANSTSV | (SEQ ID NO: 240); |
| ANSVSG | (SEQ ID NO: 241); |
| ANSVSS | (SEQ ID NO: 242); |
| AQNSNV | (SEQ ID NO: 243); |
| AQNVNS | (SEQ ID NO: 244); |
| AQNVTS | (SEQ ID NO: 245); |
| AQSQSV | (SEQ ID NO: 246); |
| AQSSSV | (SEQ ID NO: 247); |
| AQSTSV | (SEQ ID NO: 248); |
| AQSVNS | (SEQ ID NO: 249); |
| AQSVQS | (SEQ ID NO: 250); |
| AQSVSQ | (SEQ ID NO: 251); |
| AQSVSS | (SEQ ID NO: 252); |
| AQSVST | (SEQ ID NO: 253); |
| ASNNNV | (SEQ ID NO: 254); |
| ASNQNQ | (SEQ ID NO: 255); |
| ASNQNV | (SEQ ID NO: 256); |
| ASNQTQ | (SEQ ID NO: 257); |
| ASNSNV | (SEQ ID NO: 258); |
| ASNSTV | (SEQ ID NO: 259); |
| ASNTNS | (SEQ ID NO: 260); |
| ASNTNV | (SEQ ID NO: 261); |
| ASNTSV | (SEQ ID NO: 262); |
| ASNVNG | (SEQ ID NO: 263); |
| ASNVNQ | (SEQ ID NO: 264); |
| ASNVNS | (SEQ ID NO: 265); |
| ASNVNT | (SEQ ID NO: 266); |
| ASNVTG | (SEQ ID NO: 267); |
| ASNVTT | (SEQ ID NO: 268); |
| ASSNSV | (SEQ ID NO: 269); |
| ASSVSG | (SEQ ID NO: 270); |
| ASSVSN | (SEQ ID NO: 271); |
| ATNVNS | (SEQ ID NO: 272); |
| ATNVTS | (SEQ ID NO: 273); |
| ATSQSQ | (SEQ ID NO: 274); |
| ATSQSV | (SEQ ID NO: 275); |
| ATSTSG | (SEQ ID NO: 276); |
| ATSTSV | (SEQ ID NO: 277); |
| ATSVSG | (SEQ ID NO: 278); |
| ATSVSS | (SEQ ID NO: 279); |
| AVNQNS | (SEQ ID NO: 280); |
| AVNQSQ | (SEQ ID NO: 281); |
| AVNSNG | (SEQ ID NO: 282); |
| AVNSNS | (SEQ ID NO: 283); |
| AVNSNT | (SEQ ID NO: 284); |
| AVNTNS | (SEQ ID NO: 285); |
| AVSNSG | (SEQ ID NO: 286); |
| AVSNSS | (SEQ ID NO: 287); |
| AVSQNQ | (SEQ ID NO: 288); |
| AVSQSG | (SEQ ID NO: 289); |
| AVSQSQ | (SEQ ID NO: 290); |
| AVSQTQ | (SEQ ID NO: 291); |
| AVSSNQ | (SEQ ID NO: 292); |
| AVSSNS | (SEQ ID NO: 293); |
| AVSSSQ | (SEQ ID NO: 294); |
| AVSTSG | (SEQ ID NO: 295); |
| GANTVS | (SEQ ID NO: 296); |
| GAQTSS | (SEQ ID NO: 297); |
| GASNQS | (SEQ ID NO: 298); |
| GASQQS | (SEQ ID NO: 299); |
| GASSQQ | (SEQ ID NO: 300); |
| GGQVTS | (SEQ ID NO: 301); |
| GGSNQV | (SEQ ID NO: 302); |
| GNNVQS | (SEQ ID NO: 303); |
| GNQVTS | (SEQ ID NO: 304); |
| GNSNQV | (SEQ ID NO: 305); |
| GNSQQQ | (SEQ ID NO: 306); |
| GNSQQS | (SEQ ID NO: 307); |
| GNSQQV | (SEQ ID NO: 308); |
| GNSSTV | (SEQ ID NO: 309); |
| GNSTQS | (SEQ ID NO: 310); |
| GNSTQV | (SEQ ID NO: 311); |
| GNSTVS | (SEQ ID NO: 312); |
| GNSVQS | (SEQ ID NO: 313); |
| GNSVSS | (SEQ ID NO: 314); |
| GNSVST | (SEQ ID NO: 315); |
| GNSVTS | (SEQ ID NO: 316); |
| GQNTVS | (SEQ ID NO: 317); |
| GQNVAS | (SEQ ID NO: 318); |
| GQNVQS | (SEQ ID NO: 319); |
| GQNVSS | (SEQ ID NO: 320); |
| GQNVTS | (SEQ ID NO: 321); |
| GQQQSQ | (SEQ ID NO: 322); |
| GQQTSS | (SEQ ID NO: 323); |
| GQQTSV | (SEQ ID NO: 324); |
| GQQVAS | (SEQ ID NO: 325); |
| GQQVNS | (SEQ ID NO: 326); |
| GQQVQS | (SEQ ID NO: 327); |
| GQQVSG | (SEQ ID NO: 328); |
| GQQVSQ | (SEQ ID NO: 329); |
| GQQVSS | (SEQ ID NO: 330); |
| GQQVST | (SEQ ID NO: 331); |
| GQQVTS | (SEQ ID NO: 332); |
| GQSGQV | (SEQ ID NO: 333); |
| GQSNQA | (SEQ ID NO: 334); |
| GQSNQS | (SEQ ID NO: 335); |
| GQSNQV | (SEQ ID NO: 336); |
| GQSQAQ | (SEQ ID NO: 337); |
| GQSQQQ | (SEQ ID NO: 338); |
| GQSQQS | (SEQ ID NO: 339); |
| GQSQSQ | (SEQ ID NO: 340); |
| GQSSQQ | (SEQ ID NO: 341); |
| GQSSQS | (SEQ ID NO: 342); |
| GQSSQV | (SEQ ID NO: 343); |
| GQSTQS | (SEQ ID NO: 344); |
| GQSTQV | (SEQ ID NO: 345); |
| GQSVAG | (SEQ ID NO: 346); |
| GQSVAQ | (SEQ ID NO: 347); |
| GQSVAS | (SEQ ID NO: 348); |
| GQSVQN | (SEQ ID NO: 349); |
| GQSVQQ | (SEQ ID NO: 350); |
| GQSVQS | (SEQ ID NO: 351); |
| GQSVSG | (SEQ ID NO: 352); |
| GQSVSN | (SEQ ID NO: 353); |
| GQSVSQ | (SEQ ID NO: 354); |
| GQSVSS | (SEQ ID NO: 355); |
| GQSVST | (SEQ ID NO: 356); |
| GQSVTS | (SEQ ID NO: 357); |
| GSNQQV | (SEQ ID NO: 358); |
| GSNQVQ | (SEQ ID NO: 359); |
| GSNSTV | (SEQ ID NO: 360); |
| GSNSVQ | (SEQ ID NO: 361); |
| GSNSVT | (SEQ ID NO: 362); |
| GSNTAV | (SEQ ID NO: 363); |
| GSNTQV | (SEQ ID NO: 364); |
| GSNTVA | (SEQ ID NO: 365); |
| GSNTVS | (SEQ ID NO: 366); |
| GSNVAS | (SEQ ID NO: 367); |
| GSNVQQ | (SEQ ID NO: 368); |
| GSNVQS | (SEQ ID NO: 369); |
| GSNVQT | (SEQ ID NO: 370); |
| GSNVTS | (SEQ ID NO: 371); |
| GSQQSV | (SEQ ID NO: 372); |
| GSQQTQ | (SEQ ID NO: 373); |
| GSQQTV | (SEQ ID NO: 374); |
| GSQTSS | (SEQ ID NO: 375); |
| GSQTSV | (SEQ ID NO: 376); |
| GSQVAS | (SEQ ID NO: 377); |
| GSQVNS | (SEQ ID NO: 378); |
| GSQVQS | (SEQ ID NO: 379); |
| GSQVSS | (SEQ ID NO: 380); |
| GSQVST | (SEQ ID NO: 381); |
| GSQVTG | (SEQ ID NO: 382); |
| GSQVTS | (SEQ ID NO: 383); |
| GSSAQS | (SEQ ID NO: 384); |
| GSSGQV | (SEQ ID NO: 385); |
| GSSNQA | (SEQ ID NO: 386); |
| GSSNQT | (SEQ ID NO: 387); |
| GSSNQV | (SEQ ID NO: 388); |
| GSSNSV | (SEQ ID NO: 389); |
| GSSNTV | (SEQ ID NO: 390); |
| GSSNVT | (SEQ ID NO: 391); |
| GSSQAQ | (SEQ ID NO: 392); |
| GSSQQA | (SEQ ID NO: 393); |
| GSSQQV | (SEQ ID NO: 394); |
| GSSQSQ | (SEQ ID NO: 395); |
| GSSSQQ | (SEQ ID NO: 396); |
| GSSSQS | (SEQ ID NO: 397); |
| GSSSQV | (SEQ ID NO: 398); |
| GSSTQA | (SEQ ID NO: 399); |
| GSSTQG | (SEQ ID NO: 400); |
| GSSTQT | (SEQ ID NO: 401); |
| GSSTQV | (SEQ ID NO: 402); |
| GSSTSV | (SEQ ID NO: 403); |
| GSSTVN | (SEQ ID NO: 404); |
| GSSVNS | (SEQ ID NO: 405); |
| GSSVQN | (SEQ ID NO: 406); |
| GSSVQT | (SEQ ID NO: 407); |
| GSSVSG | (SEQ ID NO: 408); |
| GSSVSS | (SEQ ID NO: 409); |
| GSSVST | (SEQ ID NO: 410); |
| GSSVTN | (SEQ ID NO: 411); |
| GTNSSV | (SEQ ID NO: 412); |
| GTNSVS | (SEQ ID NO: 413); |
| GTNTVS | (SEQ ID NO: 414); |
| GTNVQS | (SEQ ID NO: 415); |
| GTNVSS | (SEQ ID NO: 416); |
| GTNVTS | (SEQ ID NO: 417); |
| GTQQSQ | (SEQ ID NO: 418); |
| GTQSTS | (SEQ ID NO: 419); |
| GTQSTV | (SEQ ID NO: 420); |
| GTQTSA | (SEQ ID NO: 421); |
| GTQTSV | (SEQ ID NO: 422); |
| GTQVQS | (SEQ ID NO: 423); |
| GTQVSG | (SEQ ID NO: 424); |
| GTQVSN | (SEQ ID NO: 425); |
| GTQVSS | (SEQ ID NO: 426); |
| GTQVST | (SEQ ID NO: 427); |
| GTSAQS | (SEQ ID NO: 428); |
| GTSGQV | (SEQ ID NO: 429); |
| GTSNQA | (SEQ ID NO: 430); |
| GTSNQS | (SEQ ID NO: 431); |
| GTSNQT | (SEQ ID NO: 432); |
| GTSNQV | (SEQ ID NO: 433); |
| GTSNSV | (SEQ ID NO: 434); |
| GTSNVS | (SEQ ID NO: 435); |
| GTSQQA | (SEQ ID NO: 436); |
| GTSQQS | (SEQ ID NO: 437); |
| GTSQQV | (SEQ ID NO: 438); |
| GTSQSQ | (SEQ ID NO: 439); |
| GTSQSV | (SEQ ID NO: 440); |
| GTSSNV | (SEQ ID NO: 441); |
| GTSTQA | (SEQ ID NO: 442); |
| GTSTQS | (SEQ ID NO: 443); |
| GTSTQT | (SEQ ID NO: 444); |
| GTSTQV | (SEQ ID NO: 445); |
| GTSTSV | (SEQ ID NO: 446); |
| GTSVAG | (SEQ ID NO: 447); |
| GTSVAS | (SEQ ID NO: 448); |
| GTSVNS | (SEQ ID NO: 449); |
| GTSVNT | (SEQ ID NO: 450); |
| GTSVQG | (SEQ ID NO: 451); |
| GTSVQN | (SEQ ID NO: 452); |
| GTSVQQ | (SEQ ID NO: 453); |
| GTSVQS | (SEQ ID NO: 454); |
| GTSVQT | (SEQ ID NO: 455); |
| GTSVSG | (SEQ ID NO: 456); |
| GTSVSN | (SEQ ID NO: 457); |
| GTSVSS | (SEQ ID NO: 458); |
| GTSVST | (SEQ ID NO: 459); |
| GTSVTS | (SEQ ID NO: 460); |
| GVNSQS | (SEQ ID NO: 461); |
| GVNSST | (SEQ ID NO: 462); |
| GVNSTS | (SEQ ID NO: 463); |
| GVNTQS | (SEQ ID NO: 464); |
| GVNTSS | (SEQ ID NO: 465); |
| GVQNTS | (SEQ ID NO: 466); |
| GVQQSQ | (SEQ ID NO: 467); |
| GVQSNQ | (SEQ ID NO: 468); |
| GVQSNS | (SEQ ID NO: 469); |
| GVQSSG | (SEQ ID NO: 470); |
| GVQSSQ | (SEQ ID NO: 471); |
| GVQSTS | (SEQ ID NO: 472); |
| GVQSTT | (SEQ ID NO: 473); |
| GVQTNS | (SEQ ID NO: 474); |
| GVQTQS | (SEQ ID NO: 475); |
| GVQTSG | (SEQ ID NO: 476); |
| GVQTSS | (SEQ ID NO: 477); |
| GVSGQG | (SEQ ID NO: 478); |
| GVSGQT | (SEQ ID NO: 479); |
| GVSNAS | (SEQ ID NO: 480); |
| GVSNNV | (SEQ ID NO: 481); |
| GVSNQG | (SEQ ID NO: 482); |
| GVSNQN | (SEQ ID NO: 483); |
| GVSNQQ | (SEQ ID NO: 484); |
| GVSNQS | (SEQ ID NO: 485); |
| GVSNQT | (SEQ ID NO: 486); |
| GVSNSS | (SEQ ID NO: 487); |
| GVSNST | (SEQ ID NO: 488); |
| GVSNTS | (SEQ ID NO: 489); |
| GVSQAQ | (SEQ ID NO: 490); |
| GVSQNQ | (SEQ ID NO: 491); |
| GVSQNS | (SEQ ID NO: 492); |
| GVSQQG | (SEQ ID NO: 493); |
| GVSQQS | (SEQ ID NO: 494); |
| GVSQQT | (SEQ ID NO: 495); |
| GVSQSG | (SEQ ID NO: 496); |
| GVSQSQ | (SEQ ID NO: 497); |
| GVSQSS | (SEQ ID NO: 498); |
| GVSSAQ | (SEQ ID NO: 499); |
| GVSSAS | (SEQ ID NO: 500); |
| GVSSGQ | (SEQ ID NO: 501); |
| GVSSNG | (SEQ ID NO: 502); |
| GVSSNQ | (SEQ ID NO: 503); |
| GVSSNS | (SEQ ID NO: 504); |
| GVSSNT | (SEQ ID NO: 505); |
| GVSSQG | (SEQ ID NO: 506); |
| GVSSQN | (SEQ ID NO: 507); |
| GVSSQQ | (SEQ ID NO: 508); |
| GVSSSG | (SEQ ID NO: 509); |
| GVSSSQ | (SEQ ID NO: 510); |
| GVSSSS | (SEQ ID NO: 511); |
| GVSSTN | (SEQ ID NO: 512); |
| GVSTAS | (SEQ ID NO: 513); |
| GVSTNS | (SEQ ID NO: 514); |
| GVSTQG | (SEQ ID NO: 515); |
| GVSTQN | (SEQ ID NO: 516); |
| GVSTQQ | (SEQ ID NO: 517); |
| GVSTQS | (SEQ ID NO: 518); |
| GVSTQT | (SEQ ID NO: 519); |
| GVSTSN | (SEQ ID NO: 520); |
| GVSTSQ | (SEQ ID NO: 521); |
| GVSTST | (SEQ ID NO: 522); |
| GVSTST | (SEQ ID NO: 523); |
| GVTNSS | (SEQ ID NO: 524); |
| GVTSNS | (SEQ ID NO: 525); |
| GVTSSN | (SEQ ID NO: 526); |
| NGSTSV | (SEQ ID NO: 527); |
| NGSVTS | (SEQ ID NO: 528); |
| NNSVSS | (SEQ ID NO: 529); |
| NQSQSQ | (SEQ ID NO: 530); |
| NQSVSN | (SEQ ID NO: 531); |
| NQSVSQ | (SEQ ID NO: 532); |
| NQSVSS | (SEQ ID NO: 533); |
| NSSNSV | (SEQ ID NO: 534); |
| NSSQSQ | (SEQ ID NO: 535); |
| NSSTVG | (SEQ ID NO: 536); |
| NSSVSG | (SEQ ID NO: 537); |
| NSSVSN | (SEQ ID NO: 538); |
| NSSVSS | (SEQ ID NO: 539); |
| NSSVTG | (SEQ ID NO: 540); |
| NTNVNS | (SEQ ID NO: 541); |
| NTQVSS | (SEQ ID NO: 542); |
| NTSGSV | (SEQ ID NO: 543); |
| NTSQSQ | (SEQ ID NO: 544); |
| NTSTSS | (SEQ ID NO: 545); |
| NTSTSV | (SEQ ID NO: 546); |
| NTSVSS | (SEQ ID NO: 547); |
| NVSGST | (SEQ ID NO: 548); |
| NVSNSG | (SEQ ID NO: 549); |
| NVSQSG | (SEQ ID NO: 550); |
| NVSQSQ | (SEQ ID NO: 551); |
| NVSQSS | (SEQ ID NO: 552); |
| NVSSSG | (SEQ ID NO: 553); |
| NVSSSS | (SEQ ID NO: 554); |
| NVSSTG | (SEQ ID NO: 555); |
| NVSTSN | (SEQ ID NO: 556); |
| NVSTSS | (SEQ ID NO: 557); |
| NVSTST | (SEQ ID NO: 558); |
| QNSTSV | (SEQ ID NO: 559); |
| QNSVSG | (SEQ ID NO: 560); |
| QQSQSQ | (SEQ ID NO: 561); |
| QQSVAG | (SEQ ID NO: 562); |
| QQSVAQ | (SEQ ID NO: 563); |
| QQSVAS | (SEQ ID NO: 564); |
| QQSVAT | (SEQ ID NO: 565); |
| QQSVSG | (SEQ ID NO: 566); |
| QQSVSN | (SEQ ID NO: 567); |
| QQSVSS | (SEQ ID NO: 568); |
| QSNQVQ | (SEQ ID NO: 569); |
| QSNTAV | (SEQ ID NO: 570); |
| QSNTNV | (SEQ ID NO: 571); |
| QSSNSV | (SEQ ID NO: 572); |
| QSSQAQ | (SEQ ID NO: 573); |
| QSSQSQ | (SEQ ID NO: 574); |
| QSSQSV | (SEQ ID NO: 575); |
| QSSTSV | (SEQ ID NO: 576); |
| QSSVSG | (SEQ ID NO: 577); |
| QSSVSN | (SEQ ID NO: 578); |
| QSSVSQ | (SEQ ID NO: 579); |
| QSSVSS | (SEQ ID NO: 580); |
| QSSVTG | (SEQ ID NO: 581); |
| QSSVTS | (SEQ ID NO: 582); |
| QTSQSQ | (SEQ ID NO: 583); |
| QTSSSQ | (SEQ ID NO: 584); |
| QTSSSV | (SEQ ID NO: 585); |
| QTSVAG | (SEQ ID NO: 586); |
| QTSVAS | (SEQ ID NO: 587); |
| QTSVSG | (SEQ ID NO: 588); |
| QTSVSN | (SEQ ID NO: 589); |
| QTSVSS | (SEQ ID NO: 590); |
| QTSVST | (SEQ ID NO: 591); |
| QTSVTS | (SEQ ID NO: 592); |
| QVSNSG | (SEQ ID NO: 593); |
| QVSNSS | (SEQ ID NO: 594); |
| QVSNST | (SEQ ID NO: 595); |
| QVSQAQ | (SEQ ID NO: 596); |
| QVSQTG | (SEQ ID NO: 597); |
| QVSQTQ | (SEQ ID NO: 598); |
| QVSSAQ | (SEQ ID NO: 599); |
| QVSSSG | (SEQ ID NO: 600); |
| QVSSSQ | (SEQ ID NO: 601); |
| QVSSSS | (SEQ ID NO: 602); |
| QVSSTQ | (SEQ ID NO: 603); |
| QVSTSG | (SEQ ID NO: 604); |
| QVSTSN | (SEQ ID NO: 605); |
| QVSTSS | (SEQ ID NO: 606); |
| QVSTST | (SEQ ID NO: 607); |
| SAQQSQ | (SEQ ID NO: 608); |
| SAQQTQ | (SEQ ID NO: 609); |
| SASQQQ | (SEQ ID NO: 610); |
| SASQSQ | (SEQ ID NO: 611); |
| SGNSTV | (SEQ ID NO: 612); |
| SGNTSV | (SEQ ID NO: 613); |
| SGNVST | (SEQ ID NO: 614); |
| SGNVTS | (SEQ ID NO: 615); |
| SGQQTQ | (SEQ ID NO: 616); |
| SGQQTV | (SEQ ID NO: 617); |
| SGQTSV | (SEQ ID NO: 618); |
| SGQVSS | (SEQ ID NO: 619); |
| SGQVTG | (SEQ ID NO: 620); |
| SGQVTQ | (SEQ ID NO: 621); |
| SGQVTT | (SEQ ID NO: 622); |
| SGSNTV | (SEQ ID NO: 623); |
| SGSTNV | (SEQ ID NO: 624); |
| SGSTQV | (SEQ ID NO: 625); |
| SGSVAS | (SEQ ID NO: 626); |
| SGSVNT | (SEQ ID NO: 627); |
| SGSVQG | (SEQ ID NO: 628); |
| SGSVQS | (SEQ ID NO: 629); |
| SNNQTV | (SEQ ID NO: 630); |
| SNNTAV | (SEQ ID NO: 631); |
| SNNTNV | (SEQ ID NO: 632); |
| SNNTQV | (SEQ ID NO: 633); |
| SNQQSQ | (SEQ ID NO: 634); |
| SNQQSV | (SEQ ID NO: 635); |
| SNQQTQ | (SEQ ID NO: 636); |
| SNQQTV | (SEQ ID NO: 637); |
| SNQSTV | (SEQ ID NO: 638); |
| SNQTSV | (SEQ ID NO: 639); |
| SNQVSG | (SEQ ID NO: 640); |
| SNQVSQ | (SEQ ID NO: 641); |
| SNQVSS | (SEQ ID NO: 642); |
| SNQVST | (SEQ ID NO: 643); |
| SNQVTG | (SEQ ID NO: 644); |
| SNQVTQ | (SEQ ID NO: 645); |
| SNQVTS | (SEQ ID NO: 646); |
| SNSGAV | (SEQ ID NO: 647); |
| SNSNAV | (SEQ ID NO: 648); |
| SNSNGV | (SEQ ID NO: 649); |
| SNSNQS | (SEQ ID NO: 650); |
| SNSQAG | (SEQ ID NO: 651); |
| SNSQAQ | (SEQ ID NO: 652); |
| SNSQAS | (SEQ ID NO: 653); |
| SNSQAT | (SEQ ID NO: 654); |
| SNSQAV | (SEQ ID NO: 655); |
| SNSQGQ | (SEQ ID NO: 656); |
| SNSQGV | (SEQ ID NO: 657); |
| SNSQQA | (SEQ ID NO: 658); |
| SNSQQG | (SEQ ID NO: 659); |
| SNSQQQ | (SEQ ID NO: 660); |
| SNSQQS | (SEQ ID NO: 661); |
| SNSQQT | (SEQ ID NO: 662); |
| SNSQQV | (SEQ ID NO: 663); |
| SNSQSG | (SEQ ID NO: 664); |
| SNSQST | (SEQ ID NO: 665); |
| SNSQSV | (SEQ ID NO: 666); |
| SNSSAV | (SEQ ID NO: 667); |
| SNSSGV | (SEQ ID NO: 668); |
| SNSSQG | (SEQ ID NO: 669); |
| SNSSQQ | (SEQ ID NO: 670); |
| SNSSQT | (SEQ ID NO: 671); |
| SNSSQV | (SEQ ID NO: 672); |
| SNSSSQ | (SEQ ID NO: 673); |
| SNSSSV | (SEQ ID NO: 674); |
| SNSTAG | (SEQ ID NO: 675); |
| SNSTGS | (SEQ ID NO: 676); |
| SNSTNV | (SEQ ID NO: 677); |
| SNSTQA | (SEQ ID NO: 678); |
| SNSTQQ | (SEQ ID NO: 679); |
| SNSTQS | (SEQ ID NO: 680); |
| SNSTQV | (SEQ ID NO: 681); |
| SNSTSA | (SEQ ID NO: 682); |
| SNSTSV | (SEQ ID NO: 683); |
| SNSTVG | (SEQ ID NO: 684); |
| SNSVAQ | (SEQ ID NO: 685); |
| SNSVAS | (SEQ ID NO: 686); |
| SNSVAT | (SEQ ID NO: 687); |
| SNSVGQ | (SEQ ID NO: 688); |
| SNSVGS | (SEQ ID NO: 689); |
| SNSVGT | (SEQ ID NO: 690); |
| SNSVNS | (SEQ ID NO: 691); |
| SNSVNT | (SEQ ID NO: 692); |
| SNSVQQ | (SEQ ID NO: 693); |
| SNSVQS | (SEQ ID NO: 694); |
| SNSVSG | (SEQ ID NO: 695); |
| SNSVSQ | (SEQ ID NO: 696); |
| SNSVSS | (SEQ ID NO: 697); |
| SNSVST | (SEQ ID NO: 698); |
| SNSVTG | (SEQ ID NO: 699); |
| SQNVAS | (SEQ ID NO: 700); |
| SQNVNS | (SEQ ID NO: 701); |
| SQQQSQ | (SEQ ID NO: 702); |
| SQQQSV | (SEQ ID NO: 703); |
| SQQQTQ | (SEQ ID NO: 704); |
| SQQQTV | (SEQ ID NO: 705); |
| SQQSTV | (SEQ ID NO: 706); |
| SQQVSG | (SEQ ID NO: 707); |
| SQQVSN | (SEQ ID NO: 708); |
| SQQVSS | (SEQ ID NO: 709); |
| SQQVST | (SEQ ID NO: 710); |
| SQQVTG | (SEQ ID NO: 711); |
| SQQVTN | (SEQ ID NO: 712); |
| SQQVTS | (SEQ ID NO: 713); |
| SQQVTT | (SEQ ID NO: 714); |
| SQSNAS | (SEQ ID NO: 715); |
| SQSNAV | (SEQ ID NO: 716); |
| SQSNGV | (SEQ ID NO: 717); |
| SQSNQT | (SEQ ID NO: 718); |
| SQSNQV | (SEQ ID NO: 719); |
| SQSNSV | (SEQ ID NO: 720); |
| SQSQAQ | (SEQ ID NO: 721); |
| SQSQAS | (SEQ ID NO: 722); |
| SQSQAV | (SEQ ID NO: 723); |
| SQSQQQ | (SEQ ID NO: 724); |
| SQSQQV | (SEQ ID NO: 725); |
| SQSQSQ | (SEQ ID NO: 726); |
| SQSSAV | (SEQ ID NO: 727); |
| SQSSGV | (SEQ ID NO: 728); |
| SQSSQG | (SEQ ID NO: 729); |
| SQSSQV | (SEQ ID NO: 730); |
| SQSSSQ | (SEQ ID NO: 731); |
| SQSSSV | (SEQ ID NO: 732); |
| SQSTAV | (SEQ ID NO: 733); |
| SQSTQS | (SEQ ID NO: 734); |
| SQSTSG | (SEQ ID NO: 735); |
| SQSTSV | (SEQ ID NO: 736); |
| SQSVAG | (SEQ ID NO: 737); |
| SQSVAN | (SEQ ID NO: 738); |
| SQSVAQ | (SEQ ID NO: 739); |
| SQSVAS | (SEQ ID NO: 740); |
| SQSVAT | (SEQ ID NO: 741); |
| SQSVGG | (SEQ ID NO: 742); |
| SQSVGN | (SEQ ID NO: 743); |
| SQSVGQ | (SEQ ID NO: 744); |
| SQSVGS | (SEQ ID NO: 745); |
| SQSVGT | (SEQ ID NO: 746); |
| SQSVNG | (SEQ ID NO: 747); |
| SQSVNS | (SEQ ID NO: 748); |
| SQSVQG | (SEQ ID NO: 749); |
| SQSVQN | (SEQ ID NO: 750); |
| SQSVQQ | (SEQ ID NO: 751); |
| SQSVQS | (SEQ ID NO: 752); |
| SQSVQT | (SEQ ID NO: 753); |
| SQSVSG | (SEQ ID NO: 754); |
| SQSVSN | (SEQ ID NO: 755); |
| SQSVSS | (SEQ ID NO: 756); |
| SQSVST | (SEQ ID NO: 757); |
| SSNGTV | (SEQ ID NO: 758); |
| SSNQNQ | (SEQ ID NO: 759); |
| SSNQNV | (SEQ ID NO: 760); |
| SSNQTQ | (SEQ ID NO: 761); |
| SSNQTV | (SEQ ID NO: 762); |
| SSNSTV | (SEQ ID NO: 763); |
| SSNTNV | (SEQ ID NO: 764); |
| SSNTQV | (SEQ ID NO: 765); |
| SSNTVG | (SEQ ID NO: 766); |
| SSNVAQ | (SEQ ID NO: 767); |
| SSNVAS | (SEQ ID NO: 768); |
| SSNVGT | (SEQ ID NO: 769); |
| SSNVNG | (SEQ ID NO: 770); |
| SSNVNQ | (SEQ ID NO: 771); |
| SSNVNS | (SEQ ID NO: 772); |
| SSNVNT | (SEQ ID NO: 773); |
| SSNVQS | (SEQ ID NO: 774); |
| SSNVQT | (SEQ ID NO: 775); |
| SSNVTG | (SEQ ID NO: 776); |
| SSNVTQ | (SEQ ID NO: 777); |
| SSNVTS | (SEQ ID NO: 778); |
| SSQNTV | (SEQ ID NO: 779); |
| SSQQSV | (SEQ ID NO: 780); |
| SSQQTQ | (SEQ ID NO: 781); |
| SSQQTV | (SEQ ID NO: 782); |
| SSQSTQ | (SEQ ID NO: 783); |
| SSQTSV | (SEQ ID NO: 784); |
| SSQVNS | (SEQ ID NO: 785); |
| SSQVSN | (SEQ ID NO: 786); |
| SSQVSQ | (SEQ ID NO: 787); |
| SSQVSS | (SEQ ID NO: 788); |
| SSQVST | (SEQ ID NO: 789); |
| SSQVTG | (SEQ ID NO: 790); |
| SSQVTQ | (SEQ ID NO: 791); |
| SSQVTS | (SEQ ID NO: 792); |
| SSQVTT | (SEQ ID NO: 793); |
| SSSNAV | (SEQ ID NO: 794); |
| SSSNQV | (SEQ ID NO: 795); |
| SSSNSV | (SEQ ID NO: 796); |
| SSSQAQ | (SEQ ID NO: 797); |
| SSSQNQ | (SEQ ID NO: 798); |
| SSSQQQ | (SEQ ID NO: 799); |
| SSSQQT | (SEQ ID NO: 800); |
| SSSQQV | (SEQ ID NO: 801); |
| SSSQSQ | (SEQ ID NO: 802); |
| SSSQSV | (SEQ ID NO: 803); |
| SSSSGQ | (SEQ ID NO: 804); |
| SSSSGV | (SEQ ID NO: 805); |
| SSSSNV | (SEQ ID NO: 806); |
| SSSSQQ | (SEQ ID NO: 807); |
| SSSSQS | (SEQ ID NO: 808); |
| SSSTAV | (SEQ ID NO: 809); |
| SSSTNV | (SEQ ID NO: 810); |
| SSSTSV | (SEQ ID NO: 811); |
| SSSVAQ | (SEQ ID NO: 812); |
| SSSVAS | (SEQ ID NO: 813); |
| SSSVGG | (SEQ ID NO: 814); |
| SSSVGQ | (SEQ ID NO: 815); |
| SSSVGS | (SEQ ID NO: 816); |
| SSSVNG | (SEQ ID NO: 817); |
| SSSVNS | (SEQ ID NO: 818); |
| SSSVNT | (SEQ ID NO: 819); |
| SSSVQG | (SEQ ID NO: 820); |
| SSSVQN | (SEQ ID NO: 821); |
| SSSVQS | (SEQ ID NO: 822); |
| SSSVQT | (SEQ ID NO: 823); |
| SSSVSG | (SEQ ID NO: 824); |
| SSSVSN | (SEQ ID NO: 825); |
| SSSVSQ | (SEQ ID NO: 826); |
| SSSVSS | (SEQ ID NO: 827); |
| SSSVST | (SEQ ID NO: 828); |
| STNGSV | (SEQ ID NO: 829); |
| STNSGV | (SEQ ID NO: 830); |
| STNSNV | (SEQ ID NO: 831); |
| STNTNV | (SEQ ID NO: 832); |
| STNTQV | (SEQ ID NO: 833); |
| STNVGS | (SEQ ID NO: 834); |
| STNVNG | (SEQ ID NO: 835); |
| STNVNS | (SEQ ID NO: 836); |
| STNVSG | (SEQ ID NO: 837); |
| STNVSS | (SEQ ID NO: 838); |
| STNVTG | (SEQ ID NO: 839); |
| STNVTS | (SEQ ID NO: 840); |
| STQQSG | (SEQ ID NO: 841); |
| STQQSQ | (SEQ ID NO: 842); |
| STQQSV | (SEQ ID NO: 843); |
| STQQTQ | (SEQ ID NO: 844); |
| STQQTS | (SEQ ID NO: 845); |
| STQQTV | (SEQ ID NO: 846); |
| STQSNV | (SEQ ID NO: 847); |
| STQSTV | (SEQ ID NO: 848); |
| STQTSS | (SEQ ID NO: 849); |
| STQTSV | (SEQ ID NO: 850); |
| STQVNS | (SEQ ID NO: 851); |
| STQVSG | (SEQ ID NO: 852); |
| STQVSN | (SEQ ID NO: 853); |
| STQVST | (SEQ ID NO: 854); |
| STQVTG | (SEQ ID NO: 855); |
| STQVTN | (SEQ ID NO: 856); |
| STQVTQ | (SEQ ID NO: 857); |
| STQVTS | (SEQ ID NO: 858); |
| STQVTT | (SEQ ID NO: 859); |
| STSGNV | (SEQ ID NO: 860); |
| STSNAS | (SEQ ID NO: 861); |
| STSNGV | (SEQ ID NO: 862); |
| STSNQV | (SEQ ID NO: 863); |
| STSNSV | (SEQ ID NO: 864); |
| STSQAQ | (SEQ ID NO: 865); |
| STSQGV | (SEQ ID NO: 866); |
| STSQNQ | (SEQ ID NO: 867); |
| STSQQQ | (SEQ ID NO: 868); |
| STSQQV | (SEQ ID NO: 869); |
| STSQSQ | (SEQ ID NO: 870); |
| STSSAV | (SEQ ID NO: 871); |
| STSSGV | (SEQ ID NO: 872); |
| STSSQQ | (SEQ ID NO: 873); |
| STSSQS | (SEQ ID NO: 874); |
| STSSQV | (SEQ ID NO: 875); |
| STSSSQ | (SEQ ID NO: 876); |
| STSTAV | (SEQ ID NO: 877); |
| STSTGV | (SEQ ID NO: 878); |
| STSTQA | (SEQ ID NO: 879); |
| STSTQG | (SEQ ID NO: 880); |
| STSTQT | (SEQ ID NO: 881); |
| STSTQV | (SEQ ID NO: 882); |
| STSTSQ | (SEQ ID NO: 883); |
| STSTSV | (SEQ ID NO: 884); |
| STSVAG | (SEQ ID NO: 885); |
| STSVAN | (SEQ ID NO: 886); |
| STSVAQ | (SEQ ID NO: 887); |
| STSVAS | (SEQ ID NO: 888); |
| STSVAT | (SEQ ID NO: 889); |
| STSVGG | (SEQ ID NO: 890); |
| STSVGN | (SEQ ID NO: 891); |
| STSVGQ | (SEQ ID NO: 892); |
| STSVGS | (SEQ ID NO: 893); |
| STSVNG | (SEQ ID NO: 894); |
| STSVNN | (SEQ ID NO: 895); |
| STSVNS | (SEQ ID NO: 896); |
| STSVQG | (SEQ ID NO: 897); |
| STSVQN | (SEQ ID NO: 898); |
| STSVQQ | (SEQ ID NO: 899); |
| STSVQS | (SEQ ID NO: 900); |
| STSVQT | (SEQ ID NO: 901); |
| STSVSG | (SEQ ID NO: 902); |
| STSVSN | (SEQ ID NO: 903); |
| STSVSQ | (SEQ ID NO: 904); |
| STSVSS | (SEQ ID NO: 905); |
| STSVST | (SEQ ID NO: 906); |
| SVNGST | (SEQ ID NO: 907); |
| SVNGTS | (SEQ ID NO: 908); |
| SVNQAQ | (SEQ ID NO: 909); |
| SVNQQQ | (SEQ ID NO: 910); |
| SVNSGT | (SEQ ID NO: 911); |
| SVNSNQ | (SEQ ID NO: 912); |
| SVNSNS | (SEQ ID NO: 913); |
| SVNSTG | (SEQ ID NO: 914); |
| SVNSTS | (SEQ ID NO: 915); |
| SVNTGS | (SEQ ID NO: 916); |
| SVNTSG | (SEQ ID NO: 917); |
| SVQQSQ | (SEQ ID NO: 918); |
| SVQQST | (SEQ ID NO: 919); |
| SVQQTQ | (SEQ ID NO: 920); |
| SVQQTS | (SEQ ID NO: 921); |
| SVQQTT | (SEQ ID NO: 922); |
| SVQSNQ | (SEQ ID NO: 923); |
| SVQSNS | (SEQ ID NO: 924); |
| SVQSSG | (SEQ ID NO: 925); |
| SVQSSQ | (SEQ ID NO: 926); |
| SVQSSS | (SEQ ID NO: 927); |
| SVQSTG | (SEQ ID NO: 928); |
| SVQSTQ | (SEQ ID NO: 929); |
| SVQSTS | (SEQ ID NO: 930); |
| SVQTSG | (SEQ ID NO: 931); |
| SVQTSN | (SEQ ID NO: 932); |
| SVQTSS | (SEQ ID NO: 933); |
| SVQVSN | (SEQ ID NO: 934); |
| SVSGNT | (SEQ ID NO: 935); |
| SVSGQS | (SEQ ID NO: 936); |
| SVSNAQ | (SEQ ID NO: 937); |
| SVSNAS | (SEQ ID NO: 938); |
| SVSNGS | (SEQ ID NO: 939); |
| SVSNGT | (SEQ ID NO: 940); |
| SVSNQG | (SEQ ID NO: 941); |
| SVSNQS | (SEQ ID NO: 942); |
| SVSNQT | (SEQ ID NO: 943); |
| SVSNST | (SEQ ID NO: 944); |
| SVSNTG | (SEQ ID NO: 945); |
| SVSQAQ | (SEQ ID NO: 946); |
| SVSQAS | (SEQ ID NO: 947); |
| SVSQGQ | (SEQ ID NO: 948); |
| SVSQNQ | (SEQ ID NO: 949); |
| SVSQQG | (SEQ ID NO: 950); |
| SVSQQQ | (SEQ ID NO: 951); |
| SVSQQS | (SEQ ID NO: 952); |
| SVSQSG | (SEQ ID NO: 953); |
| SVSQSS | (SEQ ID NO: 954); |
| SVSQTQ | (SEQ ID NO: 955); |
| SVSSAQ | (SEQ ID NO: 956); |
| SVSSAS | (SEQ ID NO: 957); |
| SVSSGG | (SEQ ID NO: 958); |
| SVSSGS | (SEQ ID NO: 959); |
| SVSSGT | (SEQ ID NO: 960); |
| SVSSNG | (SEQ ID NO: 961); |
| SVSSNQ | (SEQ ID NO: 962); |
| SVSSNS | (SEQ ID NO: 963); |
| SVSSNT | (SEQ ID NO: 964); |
| SVSSQG | (SEQ ID NO: 965); |
| SVSSQQ | (SEQ ID NO: 966); |
| SVSSQS | (SEQ ID NO: 967); |
| SVSSQT | (SEQ ID NO: 968); |
| SVSSQV | (SEQ ID NO: 969); |
| SVSSSG | (SEQ ID NO: 970); |
| SVSSSG | (SEQ ID NO: 971); |
| SVSSSS | (SEQ ID NO: 972); |
| SVSTAG | (SEQ ID NO: 973); |
| SVSTAS | (SEQ ID NO: 974); |
| SVSTAT | (SEQ ID NO: 975); |
| SVSTGS | (SEQ ID NO: 976); |
| SVSTNS | (SEQ ID NO: 977); |
| SVSTNT | (SEQ ID NO: 978); |
| SVSTQQ | (SEQ ID NO: 979); |
| SVSTQS | (SEQ ID NO: 980); |
| SVSTQT | (SEQ ID NO: 981); |
| SVSTSG | (SEQ ID NO: 982); |
| SVSTSN | (SEQ ID NO: 983); |
| SVSTSS | (SEQ ID NO: 984); |
| SVSTST | (SEQ ID NO: 985); |
| TASQSQ | (SEQ ID NO: 986); |
| TASQVQ | (SEQ ID NO: 987); |
| TGSNSV | (SEQ ID NO: 988); |
| TGSNVS | (SEQ ID NO: 989); |
| TGSSNV | (SEQ ID NO: 990); |
| TGSVNS | (SEQ ID NO: 991); |
| TGSVSN | (SEQ ID NO: 992); |
| TNSGSV | (SEQ ID NO: 993); |
| TNSGVS | (SEQ ID NO: 994); |
| TNSQVQ | (SEQ ID NO: 995); |
| TNSSGV | (SEQ ID NO: 996); |
| TNSVGS | (SEQ ID NO: 997); |
| TNSVSG | (SEQ ID NO: 998); |
| TQSQSQ | (SEQ ID NO: 999); |
| TQSQVQ | (SEQ ID NO: 1000); |
| TQSTVS | (SEQ ID NO: 1001); |
| TQSVSN | (SEQ ID NO: 1002); |
| TQSVSS | (SEQ ID NO: 1003); |
| TSNGVS | (SEQ ID NO: 1004); |
| TSNVGS | (SEQ ID NO: 1005); |
| TSNVSG | (SEQ ID NO: 1006); |
| TSSGNV | (SEQ ID NO: 1007); |
| TSSGVN | (SEQ ID NO: 1008); |
| TSSNGV | (SEQ ID NO: 1009); |
| TSSNSV | (SEQ ID NO: 1010); |
| TSSNVG | (SEQ ID NO: 1011); |
| TSSQSQ | (SEQ ID NO: 1012); |
| TSSSVQ | (SEQ ID NO: 1013); |
| TSSVGN | (SEQ ID NO: 1014); |
| TSSVNG | (SEQ ID NO: 1015); |
| TSSVSG | (SEQ ID NO: 1016); |
| TSSVSS | (SEQ ID NO: 1017); |
| TSSVTS | (SEQ ID NO: 1018); |
| TVNSGS | (SEQ ID NO: 1019); |
| TVNSSG | (SEQ ID NO: 1020); |
| TVSGNS | (SEQ ID NO: 1021); |
| TVSGSN | (SEQ ID NO: 1022); |
| TVSNGS | (SEQ ID NO: 1023); |
| TVSNSG | (SEQ ID NO: 1024); |
| TVSNST | (SEQ ID NO: 1025); |
| TVSNVG | (SEQ ID NO: 1026); |
| TVSQNQ | (SEQ ID NO: 1027); |
| TVSQQV | (SEQ ID NO: 1028); |
| TVSQSG | (SEQ ID NO: 1029); |
| TVSQSQ | (SEQ ID NO: 1030); |
| TVSSGN | (SEQ ID NO: 1031); |
| TVSSNG | (SEQ ID NO: 1032); |
| TVSSNQ | (SEQ ID NO: 1033); |
| TVSSNS | (SEQ ID NO: 1034); |
| TVSSSQ | (SEQ ID NO: 1035); |
| TVSTSG | (SEQ ID NO: 1036); |
| TVSTSN | (SEQ ID NO: 1037); |
| TVSTSS | (SEQ ID NO: 1038); |
| TVSTST | (SEQ ID NO: 1039); |
| VGSTNS | (SEQ ID NO: 1040); |
| VNSTSG | (SEQ ID NO: 1041); |
| VNSTSN | (SEQ ID NO: 1042); |
| VSSQSQ | (SEQ ID NO: 1043); |
| VSSQVQ | (SEQ ID NO: 1044); |
| VSSTNG | (SEQ ID NO: 1045); |
| VTSNSG | (SEQ ID NO: 1046); |
| and | |
| VTSQSQ | (SEQ ID NO: 1047). |

9. The composition according to claim 8, wherein the hexapeptide comprises 1, 2 or 3 amino acids having polar basic side chains.

10. The composition according to claim 8, wherein the hexapeptide comprises 1 amino acid having a polar acidic side chain.

11. The composition according to claim 8, wherein the hexapeptide comprises 0, 1, 2, 3, 4, or 5 amino acids having hydrophobic side chains.

12. The composition according to claim 8, wherein the hexapeptide comprises 0, 1, 2, 3, 4, or 5 amino acids having polar uncharged side chains.

13. A composition for treatment of one or more inflammatory conditions in a mammal wherein the composition comprises a fibril and a plasma protein, wherein the fibril comprises four or more hexapeptides comprising amino acids of an L-configuration, D-configuration or a mixture of configurations having free energy of formation of −23 kcal/mol or less when the hexapeptide sequence in subjected to the Rosetta-Profile algorithm.

14. The composition according to claim 13, wherein the plasma protein is selected from a group consisting of: Apolipoprotein B-100; Serum Albumin; Complement C3; Complement C1s; Beta-2-glycoprotein 1; Clusterin; Coagulation Factor V; Complement C1r; Apolipoprotein A-I; ITIH2; Complement 1qB; Apolipoprotein A-IV; Complement Factor H; Fibrinogen beta chain; Complement C4-A; Transthyretin; SerpinG1 Plasma protease C1 inhibitor; Fibrinogen alpha chain; Complement C1qA; Vitronectin; Serpina-1 Alpha-1-antitrypsin; VitaminD-binding protein; Haptoglobin-related protein; ITIH4; Fibrinogen gamma chain; SerpinC1 Antithrombin-III; Apolipoprotein A-2; Complement Factor H-related protein; Gelsolin; Complement factor B; Alpha-2-HS-glycoprotein; Serum paraoxinase/arylesterase 1; Complement C5; Apolipoprotein C5; Apolipoprotein C-II; Apolipoprotein C-I; ITIH1; von Willebrand factor; Ceruloplasmin; Apolipoprotein E; Filamin-A; Histidine-rich glycoprotein; SerpinF2 alpha-2-antiplasmin; Coagulation factor II Prothrombin; Coagulation factor X; Vitamin K-dependent protein; Apolipoprotein C-III; Alpha-1-acid glycoprotein 2; Coagulation factor DC; Apolipoprotein M.


SEQUENCE LISTING (1061 sequences)

SEQ ID NO: 1 - protein, 6 aa, Artificial Sequence
  Synthetic peptide
SVNVDL

SEQ ID NO: 2 - protein, 6 aa, Artificial Sequence
  Synthetic peptide
SLNVDV

SEQ ID NO: 3 - protein, 6 aa, Artificial Sequence
  Synthetic peptide
SVDVNL

SEQ ID NO: 4 - protein, 6 aa, Artificial Sequence
  Synthetic peptide
DLSVVL

SEQ ID NO: 5 - protein, 6 aa, Artificial Sequence
  Synthetic peptide
SVNLDV

SEQ ID NO: 6 - protein, 6 aa, Artificial Sequence
  Synthetic peptide
SVVNDV

SEQ ID NO: 7 - protein, 6 aa, Artificial Sequence
  Synthetic peptide
DVSLVN

SEQ ID NO: 8 - protein, 6 aa, Artificial Sequence
  Synthetic peptide
DVSVLN

SEQ ID NO: 9 - protein, 6 aa, Artificial Sequence
  Synthetic peptide
SDLVNV

SEQ ID NO: 10 - protein, 6 aa, Artificial Sequence
  Synthetic peptide
SLNVVS

SEQ ID NO: 11 - protein, 6 aa, Artificial Sequence
  Synthetic peptide
LNVDSV

SEQ ID NO: 12 - protein, 6 aa, Artificial Sequence
  Synthetic peptide
NDLSVV

SEQ ID NO: 13 - protein, 6 aa, Artificial Sequence
  Synthetic peptide
VDNLVS

SEQ ID NO: 14 - protein, 6 aa, Artificial Sequence
  Synthetic peptide
VNDVSL

SEQ ID NO: 15 - protein, 6 aa, Artificial Sequence
  Synthetic peptide
VSDNVL

SEQ ID NO: 16 - protein, 6 aa, Artificial Sequence
  Synthetic peptide
LNDVVS

SEQ ID NO: 17 - protein, 6 aa, Artificial Sequence
  Synthetic peptide
LSVDVN

SEQ ID NO: 18 - protein, 6 aa, Artificial Sequence
  Synthetic peptide
NSVDLV

SEQ ID NO: 19 - protein, 6 aa, Artificial Sequence
  Synthetic peptide
VDVLNS

SEQ ID NO: 20 - protein, 6 aa, Artificial Sequence
  Synthetic peptide
VNDSVL

SEQ ID NO: 21 - protein, 6 aa, Artificial Sequence
  Synthetic peptide
VNVSLD

SEQ ID NO: 22 - protein, 6 aa, Artificial Sequence
  Synthetic peptide
LDVNSV

SEQ ID NO: 23 - protein, 6 aa, Artificial Sequence
  Synthetic peptide
LNVVDS

SEQ ID NO: 24 - protein, 6 aa, Artificial Sequence
  Synthetic peptide
LSDVVN

SEQ ID NO: 25 - protein, 6 aa, Artificial Sequence
  Synthetic peptide
LVVNDS

SEQ ID NO: 26 - protein, 6 aa, Artificial Sequence
  Synthetic peptide
NSLDVV

SEQ ID NO: 27 - protein, 6 aa, Artificial Sequence
  Synthetic peptide
VLVDNS

SEQ ID NO: 28 - protein, 6 aa, Artificial Sequence
  Synthetic peptide
VNLSDV

SEQ ID NO: 29 - protein, 6 aa, Artificial Sequence
  Synthetic peptide
VNVDLS

SEQ ID NO: 30 - protein, 6 aa, Artificial Sequence
  Synthetic peptide
DNVSVD

SEQ ID NO: 31 - protein, 6 aa, Artificial Sequence
  Synthetic peptide
LSDNVV

SEQ ID NO: 32 - protein, 6 aa, Artificial Sequence
  Synthetic peptide
LSVVDN

SEQ ID NO: 33 - protein, 6 aa, Artificial Sequence
  Synthetic peptide
VLDVSN

SEQ ID NO: 34 - protein, 6 aa, Artificial Sequence
  Synthetic peptide
VNDLVS

SEQ ID NO: 35 - protein, 6 aa, Artificial Sequence
  Synthetic peptide
VNVSDL

SEQ ID NO: 36 - protein, 6 aa, Artificial Sequence
  Synthetic peptide
VVLSDN

SEQ ID NO: 37 - protein, 6 aa, Artificial Sequence
  Synthetic peptide
LVSVNL

SEQ ID NO: 38 - protein, 6 aa, Artificial Sequence
  Synthetic peptide
LVNVSV

SEQ ID NO: 39 - protein, 6 aa, Artificial Sequence
  Synthetic peptide
SLNVSV

SEQ ID NO: 40 - protein, 6 aa, Artificial Sequence
  Synthetic peptide
LVSVNS

SEQ ID NO: 41 - protein, 6 aa, Artificial Sequence
  Synthetic peptide
SVDVNV

SEQ ID NO: 42 - protein, 6 aa, Artificial Sequence
  Synthetic peptide
LVVSVL

SEQ ID NO: 43 - protein, 6 aa, Artificial Sequence
  Synthetic peptide
VNLVVS

SEQ ID NO: 44 - protein, 6 aa, Artificial Sequence
  Synthetic peptide
SNLVSV

SEQ ID NO: 45 - protein, 6 aa, Artificial Sequence
  Synthetic peptide
SVNVLS

SEQ ID NO: 46 - protein, 6 aa, Artificial Sequence
  Synthetic peptide
VLVSVL

SEQ ID NO: 47 - protein, 6 aa, Artificial Sequence
  Synthetic peptide
LVNVSL

SEQ ID NO: 48 - protein, 6 aa, Artificial Sequence
  Synthetic peptide
SVNVDS

SEQ ID NO: 49 - protein, 6 aa, Artificial Sequence
  Synthetic peptide
VLSVNV

SEQ ID NO: 50 - protein, 6 aa, Artificial Sequence
  Synthetic peptide
NLVVSV

SEQ ID NO: 51 - protein, 6 aa, Artificial Sequence
  Synthetic peptide
SVVLNV

SEQ ID NO: 52 - protein, 6 aa, Artificial Sequence
  Synthetic peptide
LSVVNL

SEQ ID NO: 53 - protein, 6 aa, Artificial Sequence
  Synthetic peptide
SNLVVS

SEQ ID NO: 54 - protein, 6 aa, Artificial Sequence
  Synthetic peptide
SVVLDV

SEQ ID NO: 55 - protein, 6 aa, Artificial Sequence
  Synthetic peptide
LVSLNV

SEQ ID NO: 56 - protein, 6 aa, Artificial Sequence
  Synthetic peptide
VSLNVV

SEQ ID NO: 57 - protein, 6 aa, Artificial Sequence
  Synthetic peptide
VKVQIY

SEQ ID NO: 58 - protein, 6 aa, Artificial Sequence
  Synthetic peptide
QVVIYK

SEQ ID NO: 59 - protein, 6 aa, Artificial Sequence
  Synthetic peptide
KVIQVY

SEQ ID NO: 60 - protein, 6 aa, Artificial Sequence
  Synthetic peptide
VYVKIY

SEQ ID NO: 61 - protein, 6 aa, Artificial Sequence
  Synthetic peptide
QIVVYK

SEQ ID NO: 62 - protein, 6 aa, Artificial Sequence
  Synthetic peptide
QVIKVY

SEQ ID NO: 63 - protein, 6 aa, Artificial Sequence
  Synthetic peptide
VQVKIY

SEQ ID NO: 64 - protein, 6 aa, Artificial Sequence
  Synthetic peptide
QVVKIY

SEQ ID NO: 65 - protein, 6 aa, Artificial Sequence
  Synthetic peptide
QVIVYK

SEQ ID NO: 66 - protein, 6 aa, Artificial Sequence
  Synthetic peptide
QIVKVY

SEQ ID NO: 67 - protein, 6 aa, Artificial Sequence
  Synthetic peptide
QKIVVY

SEQ ID NO: 68 - protein, 6 aa, Artificial Sequence
  Synthetic peptide
QKVVYI

SEQ ID NO: 69 - protein, 6 aa, Artificial Sequence
  Synthetic peptide
KVQVYI

SEQ ID NO: 70 - protein, 6 aa, Artificial Sequence
  Synthetic peptide
QVVKYI

SEQ ID NO: 71 - protein, 6 aa, Artificial Sequence
  Synthetic peptide
KVQIYV

SEQ ID NO: 72 - protein, 6 aa, Artificial Sequence
  Synthetic peptide
VKIQVY

SEQ ID NO: 73 - protein, 6 aa, Artificial Sequence
  Synthetic peptide
VIQKVY

SEQ ID NO: 74 - protein, 6 aa, Artificial Sequence
  Synthetic peptide
KVVIYK

SEQ ID NO: 75 - protein, 6 aa, Artificial Sequence
  Synthetic peptide
QIVKYV

SEQ ID NO: 76 - protein, 6 aa, Artificial Sequence
  Synthetic peptide
QKVIYV

SEQ ID NO: 77 - protein, 6 aa, Artificial Sequence
  Synthetic peptide
KQVVIY

SEQ ID NO: 78 - protein, 6 aa, Artificial Sequence
  Synthetic peptide
KIQVYV

SEQ ID NO: 79 - protein, 6 aa, Artificial Sequence
  Synthetic peptide
KVYVQI

SEQ ID NO: 80 - protein, 6 aa, Artificial Sequence
  Synthetic peptide
VVQIYK

SEQ ID NO: 81 - protein, 6 aa, Artificial Sequence
  Synthetic peptide
KQVIVY

SEQ ID NO: 82 - protein, 6 aa, Artificial Sequence
  Synthetic peptide
VQIKVY

SEQ ID NO: 83 - protein, 6 aa, Artificial Sequence
  Synthetic peptide
QKIVYV

SEQ ID NO: 84 - protein, 6 aa, Artificial Sequence
  Synthetic peptide
VIQVYK

SEQ ID NO: 85 - protein, 6 aa, Artificial Sequence
  Synthetic peptide
KVVIQY

SEQ ID NO: 86 - protein, 6 aa, Artificial Sequence
  Synthetic peptide
KVQVIY

SEQ ID NO: 87 - protein, 6 aa, Artificial Sequence
  Synthetic peptide
QYVVIK

SEQ ID NO: 88 - protein, 6 aa, Artificial Sequence
  Synthetic peptide
YVVIQK

SEQ ID NO: 89 - protein, 6 aa, Artificial Sequence
  Synthetic peptide
KIVVQY

SEQ ID NO: 90 - protein, 6 aa, Artificial Sequence
  Synthetic peptide
YQVIVK

SEQ ID NO: 91 - protein, 6 aa, Artificial Sequence
  Synthetic peptide
KIYVQV

SEQ ID NO: 92 - protein, 6 aa, Artificial Sequence
  Synthetic peptide
KVVIYQ

SEQ ID NO: 93 - protein, 6 aa, Artificial Sequence
  Synthetic peptide
KYVVQI

SEQ ID NO: 94 - protein, 6 aa, Artificial Sequence
  Synthetic peptide
YQIVVK

SEQ ID NO: 95 - protein, 6 aa, Artificial Sequence
  Synthetic peptide
YVVIQY

SEQ ID NO: 96 - protein, 6 aa, Artificial Sequence
  Synthetic peptide
KQVIYV

SEQ ID NO: 97 - protein, 6 aa, Artificial Sequence
  Synthetic peptide
KYVQVI

SEQ ID NO: 98 - protein, 6 aa, Artificial Sequence
  Synthetic peptide
KYVVIQ

SEQ ID NO: 99 - protein, 6 aa, Artificial Sequence
  Synthetic peptide
QKVVIY

SEQ ID NO: 100 - protein, 6 aa, Artificial Sequence
  Synthetic peptide
QYVIVK

SEQ ID NO: 101 - protein, 6 aa, Artificial Sequence
  Synthetic peptide
IVQKVY

SEQ ID NO: 102 - protein, 6 aa, Artificial Sequence
  Synthetic peptide
KYIVVQ

SEQ ID NO: 103 - protein, 6 aa, Artificial Sequence
  Synthetic peptide
QVIKYV

SEQ ID NO: 104 - protein, 6 aa, Artificial Sequence
  Synthetic peptide
YIVVQK

SEQ ID NO: 105 - protein, 6 aa, Artificial Sequence
  Synthetic peptide
YQVVIK

SEQ ID NO: 106 - protein, 6 aa, Artificial Sequence
  Synthetic peptide
IKVQVY

SEQ ID NO: 107 - protein, 6 aa, Artificial Sequence
  Synthetic peptide
KVVQYI

SEQ ID NO: 108 - protein, 6 aa, Artificial Sequence
  Synthetic peptide
KYVQIV

SEQ ID NO: 109 - protein, 6 aa, Artificial Sequence
  Synthetic peptide
VKQVYI

SEQ ID NO: 110 - protein, 6 aa, Artificial Sequence
  Synthetic peptide
VQVIYK

SEQ ID NO: 111 - protein, 6 aa, Artificial Sequence
  Synthetic peptide
VVIQKY

SEQ ID NO: 112 - protein, 6 aa, Artificial Sequence
  Synthetic peptide
IQVVYK

SEQ ID NO: 113 - protein, 6 aa, Artificial Sequence
  Synthetic peptide
KIVVYQ

SEQ ID NO: 114 - protein, 6 aa, Artificial Sequence
  Synthetic peptide
KYQVIV

SEQ ID NO: 115 - protein, 6 aa, Artificial Sequence
  Synthetic peptide
KYVIVQ

SEQ ID NO: 116 - protein, 6 aa, Artificial Sequence
  Synthetic peptide
VVYIQK

SEQ ID NO: 117 - protein, 6 aa, Artificial Sequence
  Synthetic peptide
IVQVYK

SEQ ID NO: 118 - protein, 6 aa, Artificial Sequence
  Synthetic peptide
KQIVYV

SEQ ID NO: 119 - protein, 6 aa, Artificial Sequence
  Synthetic peptide
KYVIQV

SEQ ID NO: 120 - protein, 6 aa, Artificial Sequence
  Synthetic peptide
QKVIVY

SEQ ID NO: 121 - protein, 6 aa, Artificial Sequence
  Synthetic peptide
QYIKVV

SEQ ID NO: 122 - protein, 6 aa, Artificial Sequence
  Synthetic peptide
QYIVKV

SEQ ID NO: 123 - protein, 6 aa, Artificial Sequence
  Synthetic peptide
VQIVYK

SEQ ID NO: 124 - protein, 6 aa, Artificial Sequence
  Synthetic peptide
VVQKYI

SEQ ID NO: 125 - protein, 6 aa, Artificial Sequence
  Synthetic peptide
VYQIVK

SEQ ID NO: 126 - protein, 6 aa, Artificial Sequence
  Synthetic peptide
YVQVIK

SEQ ID NO: 127 - protein, 6 aa, Artificial Sequence
  Synthetic peptide
QIVYVK

SEQ ID NO: 128 - protein, 6 aa, Artificial Sequence
  Synthetic peptide
IKVYQV

SEQ ID NO: 129 - protein, 6 aa, Artificial Sequence
  Synthetic peptide
KVVYIQ

SEQ ID NO: 130 - protein, 6 aa, Artificial Sequence
  Synthetic peptide
VQKYIV

SEQ ID NO: 131 - protein, 6 aa, Artificial Sequence
  Synthetic peptide
VQKYVI

SEQ ID NO: 132 - protein, 6 aa, Artificial Sequence
  Synthetic peptide
KVVQIY

SEQ ID NO: 133 - protein, 6 aa, Artificial Sequence
  Synthetic peptide
YVIKIY

SEQ ID NO: 134 - protein, 6 aa, Artificial Sequence
  Synthetic peptide
QVIVYV

SEQ ID NO: 135 - protein, 6 aa, Artificial Sequence
  Synthetic peptide
VVIKVY

SEQ ID NO: 136 - protein, 6 aa, Artificial Sequence
  Synthetic peptide
KVVIYI

SEQ ID NO: 137 - protein, 6 aa, Artificial Sequence
  Synthetic peptide
VYVVIK

SEQ ID NO: 138 - protein, 6 aa, Artificial Sequence
  Synthetic peptide
YIVQVY

SEQ ID NO: 139 - protein, 6 aa, Artificial Sequence
  Synthetic peptide
YVIQVY

SEQ ID NO: 140 - protein, 6 aa, Artificial Sequence
  Synthetic peptide
YVIVVY

SEQ ID NO: 141 - protein, 6 aa, Artificial Sequence
  Synthetic peptide
KVIVYI

SEQ ID NO: 142 - protein, 6 aa, Artificial Sequence
  Synthetic peptide
YVIKVY

SEQ ID NO: 143 - protein, 6 aa, Artificial Sequence
  Synthetic peptide
VQVIVK

SEQ ID NO: 144 - protein, 6 aa, Artificial Sequence
  Synthetic peptide
YVVKIY

SEQ ID NO: 145 - protein, 6 aa, Artificial Sequence
  Synthetic peptide
YVVQVI

SEQ ID NO: 146 - protein, 6 aa, Artificial Sequence
  Synthetic peptide
QIVVYQ

SEQ ID NO: 147 - protein, 6 aa, Artificial Sequence
  Synthetic peptide
VVQIVK

SEQ ID NO: 148 - protein, 6 aa, Artificial Sequence
  Synthetic peptide
VQIVVK

SEQ ID NO: 149 - protein, 6 aa, Artificial Sequence
  Synthetic peptide
VIVVYK

SEQ ID NO: 150 - protein, 6 aa, Artificial Sequence
  Synthetic peptide
IVQVYI

SEQ ID NO: 151 - protein, 6 aa, Artificial Sequence
  Synthetic peptide
YVVIQV

SEQ ID NO: 152 - protein, 6 aa, Artificial Sequence
  Synthetic peptide
VYQVVI

SEQ ID NO: 153 - protein, 6 aa, Artificial Sequence
  Synthetic peptide
KYIQVY

SEQ ID NO: 154 - protein, 6 aa, Artificial Sequence
  Synthetic peptide
KYVVIY

SEQ ID NO: 155 - protein, 6 aa, Artificial Sequence
  Synthetic peptide
KYVQIY

SEQ ID NO: 156 - protein, 6 aa, Artificial Sequence
  Synthetic peptide
VVQVIK

SEQ ID NO: 157 - protein, 6 aa, Artificial Sequence
  Synthetic peptide
QKIVVK

SEQ ID NO: 158 - protein, 6 aa, Artificial Sequence
  Synthetic peptide
KYVIVY

SEQ ID NO: 159 - protein, 6 aa, Artificial Sequence
  Synthetic peptide
KQVVIK

SEQ ID NO: 160 - protein, 6 aa, Artificial Sequence
  Synthetic peptide
YVQIYV

SEQ ID NO: 161 - protein, 6 aa, Artificial Sequence
  Synthetic peptide
YKVQVY

SEQ ID NO: 162 - protein, 6 aa, Artificial Sequence
  Synthetic peptide
IKVQIY

SEQ ID NO: 163 - protein, 6 aa, Artificial Sequence
  Synthetic peptide
KIVVQK

SEQ ID NO: 164 - protein, 6 aa, Artificial Sequence
  Synthetic peptide
VIKVVI

SEQ ID NO: 165 - protein, 6 aa, Artificial Sequence
  Synthetic peptide
VYIKVV

SEQ ID NO: 166 - protein, 6 aa, Artificial Sequence
  Synthetic peptide
VYIVQV

SEQ ID NO: 167 - protein, 6 aa, Artificial Sequence
  Synthetic peptide
IVYVQI

SEQ ID NO: 168 - protein, 6 aa, Artificial Sequence
  Synthetic peptide
KVQIYK

SEQ ID NO: 169 - protein, 6 aa, Artificial Sequence
  Synthetic peptide
IYVIVY

SEQ ID NO: 170 - protein, 6 aa, Artificial Sequence
  Synthetic peptide
VIYVIV

SEQ ID NO: 171 - protein, 6 aa, Artificial Sequence
  Synthetic peptide
KYQVYI

SEQ ID NO: 172 - protein, 6 aa, Artificial Sequence
  Synthetic peptide
VIQKVV

SEQ ID NO: 173 - protein, 6 aa, Artificial Sequence
  Synthetic peptide
VKIVYV

SEQ ID NO: 174 - protein, 6 aa, Artificial Sequence
  Synthetic peptide
VKQIVV

SEQ ID NO: 175 - protein, 6 aa, Artificial Sequence
  Synthetic peptide
YIVKQY

SEQ ID NO: 176 - protein, 6 aa, Artificial Sequence
  Synthetic peptide
YIVVQY

SEQ ID NO: 177 - protein, 6 aa, Artificial Sequence
  Synthetic peptide
YKIQVY

SEQ ID NO: 178 - protein, 6 aa, Artificial Sequence
  Synthetic peptide
YQVVIY

SEQ ID NO: 179 - protein, 6 aa, Artificial Sequence
  Synthetic peptide
YVVKQY

SEQ ID NO: 180 - protein, 6 aa, Artificial Sequence
  Synthetic peptide
VIVKVQ

SEQ ID NO: 181 - protein, 6 aa, Artificial Sequence
  Synthetic peptide
QKVVIK

SEQ ID NO: 182 - protein, 6 aa, Artificial Sequence
  Synthetic peptide
GMVVVG

SEQ ID NO: 183 - protein, 6 aa, Artificial Sequence
  Synthetic peptide
GVVVMG

SEQ ID NO: 184 - protein, 6 aa, Artificial Sequence
  Synthetic peptide
GVVMVG

SEQ ID NO: 185 - protein, 6 aa, Artificial Sequence
  Synthetic peptide
GGVVVM

SEQ ID NO: 186 - protein, 6 aa, Artificial Sequence
  Synthetic peptide
GVVGVM

SEQ ID NO: 187 - protein, 6 aa, Artificial Sequence
  Synthetic peptide
GVVVGM

SEQ ID NO: 188 - protein, 6 aa, Artificial Sequence
  Synthetic peptide
GVMVVG

SEQ ID NO: 189 - protein, 6 aa, Artificial Sequence
  Synthetic peptide
GGVVMV

SEQ ID NO: 190 - protein, 6 aa, Artificial Sequence
  Synthetic peptide
GGVMVV

SEQ ID NO: 191 - protein, 6 aa, Artificial Sequence
  Synthetic peptide
GMVVGV

SEQ ID NO: 192 - protein, 6 aa, Artificial Sequence
  Synthetic peptide
GVVMGV

SEQ ID NO: 193 - protein, 6 aa, Artificial Sequence
  Synthetic peptide
GMVGVV

SEQ ID NO: 194 - protein, 6 aa, Artificial Sequence
  Synthetic peptide
GVMVGV

SEQ ID NO: 195 - protein, 6 aa, Artificial Sequence
  Synthetic peptide
GVVGMV

SEQ ID NO: 196 - protein, 6 aa, Artificial Sequence
  Synthetic peptide
GGMVVV

SEQ ID NO: 197 - protein, 6 aa, Artificial Sequence
  Synthetic peptide
GVGVMV

SEQ ID NO: 198 - protein, 6 aa, Artificial Sequence
  Synthetic peptide
GVGVVM

SEQ ID NO: 199 - protein, 6 aa, Artificial Sequence
  Synthetic peptide
MVVVGM

SEQ ID NO: 200 - protein, 6 aa, Artificial Sequence
  Synthetic peptide
GMGVVV

SEQ ID NO: 201 - protein, 6 aa, Artificial Sequence
  Synthetic peptide
MVVVGG

SEQ ID NO: 202 - protein, 6 aa, Artificial Sequence
  Synthetic peptide
GVGMVV

SEQ ID NO: 203 - protein, 6 aa, Artificial Sequence
  Synthetic peptide
GVVGGV

SEQ ID NO: 204 - protein, 6 aa, Artificial Sequence
  Synthetic peptide
MVGVVG

SEQ ID NO: 205 - protein, 6 aa, Artificial Sequence
  Synthetic peptide
VVMVGG

SEQ ID NO: 206 - protein, 6 aa, Artificial Sequence
  Synthetic peptide
MGVVVG

SEQ ID NO: 207 - protein, 6 aa, Artificial Sequence
  Synthetic peptide
MVGVGV

SEQ ID NO: 208 - protein, 6 aa, Artificial Sequence
  Synthetic peptide
VGVMVG

SEQ ID NO: 209 - protein, 6 aa, Artificial Sequence
  Synthetic peptide
MVVGVG

SEQ ID NO: 210 - protein, 6 aa, Artificial Sequence
  Synthetic peptide
VVGVMG

SEQ ID NO: 211 - protein, 6 aa, Artificial Sequence
  Synthetic peptide
VMGVVG

SEQ ID NO: 212 - protein, 6 aa, Artificial Sequence
  Synthetic peptide
VGVGVM

SEQ ID NO: 213 - protein, 6 aa, Artificial Sequence
  Synthetic peptide
VMVGVG

SEQ ID NO: 214 - protein, 6 aa, Artificial Sequence
  Synthetic peptide
MGGVVV

SEQ ID NO: 215 - protein, 6 aa, Artificial Sequence
  Synthetic peptide
VGMVVG

SEQ ID NO: 216 - protein, 6 aa, Artificial Sequence
  Synthetic peptide
VVGVGM

SEQ ID NO: 217 - protein, 6 aa, Artificial Sequence
  Synthetic peptide
VGGVVM

SEQ ID NO: 218 - protein, 6 aa, Artificial Sequence
  Synthetic peptide
VVGMVG

SEQ ID NO: 219 - protein, 6 aa, Artificial Sequence
  Synthetic peptide
VGVVMG

SEQ ID NO: 220 - protein, 6 aa, Artificial Sequence
  Synthetic peptide
MVGGVV

SEQ ID NO: 221 - protein, 6 aa, Artificial Sequence
  Synthetic peptide
VGMVGV

SEQ ID NO: 222 - protein, 6 aa, Artificial Sequence
  Synthetic peptide
VMVVGG

SEQ ID NO: 223 - protein, 6 aa, Artificial Sequence
  Synthetic peptide
VVGGVM

SEQ ID NO: 224 - protein, 6 aa, Artificial Sequence
  Synthetic peptide
VGGVMV

SEQ ID NO: 225 - protein, 6 aa, Artificial Sequence
  Synthetic peptide
VVVGMG

SEQ ID NO: 226 - protein, 6 aa, Artificial Sequence
  Synthetic peptide
VVGMGV

SEQ ID NO: 227 - protein, 6 aa, Artificial Sequence
  Synthetic peptide
MGVGVV

SEQ ID NO: 228 - protein, 6 aa, Artificial Sequence
  Synthetic peptide
VGVVGM

SEQ ID NO: 229 - protein, 6 aa, Artificial Sequence
  Synthetic peptide
VGGMVV

SEQ ID NO: 230 - protein, 6 aa, Artificial Sequence
  Synthetic peptide
VVVMGG

SEQ ID NO: 231 - protein, 6 aa, Artificial Sequence
  Synthetic peptide
VVMGGV

SEQ ID NO: 232 - protein, 6 aa, Artificial Sequence
  Synthetic peptide
VVVGGM

SEQ ID NO: 233 - protein, 6 aa, Artificial Sequence
  Synthetic peptide
VVGGMV

SEQ ID NO: 234 - protein, 6 aa, Artificial Sequence
  Synthetic peptide
VGMGVV

SEQ ID NO: 235 - protein, 6 aa, Artificial Sequence
  Synthetic peptide
VMGGVV

SEQ ID NO: 236 - protein, 6 aa, Artificial Sequence
  Synthetic peptide
VGGVGV

SEQ ID NO: 237 - protein, 6 aa, Artificial Sequence
  Synthetic peptide
VGVMGV

SEQ ID NO: 238 - protein, 6 aa, Artificial Sequence
  Synthetic peptide
VGVGMV

SEQ ID NO: 239 - protein, 6 aa, Artificial Sequence
  Synthetic peptide
VMVGGV

SEQ ID NO: 240 - protein, 6 aa, Artificial Sequence
  Synthetic peptide
ANSTSV

SEQ ID NO: 241 - protein, 6 aa, Artificial Sequence
  Synthetic peptide
ANSVSG

SEQ ID NO: 242 - protein, 6 aa, Artificial Sequence
  Synthetic peptide
ANSVSS

SEQ ID NO: 243 - protein, 6 aa, Artificial Sequence
  Synthetic peptide
AQNSNV

SEQ ID NO: 244 - protein, 6 aa, Artificial Sequence
  Synthetic peptide
AQNVNS

SEQ ID NO: 245 - protein, 6 aa, Artificial Sequence
  Synthetic peptide
AQNVTS

SEQ ID NO: 246 - protein, 6 aa, Artificial Sequence
  Synthetic peptide
AQSQSV

SEQ ID NO: 247 - protein, 6 aa, Artificial Sequence
  Synthetic peptide
AQSSSV

SEQ ID NO: 248 - protein, 6 aa, Artificial Sequence
  Synthetic peptide
AQSTSV

SEQ ID NO: 249 - protein, 6 aa, Artificial Sequence
  Synthetic peptide
AQSVNS

SEQ ID NO: 250 - protein, 6 aa, Artificial Sequence
  Synthetic peptide
AQSVQS

SEQ ID NO: 251 - protein, 6 aa, Artificial Sequence
  Synthetic peptide
AQSVSQ

SEQ ID NO: 252 - protein, 6 aa, Artificial Sequence
  Synthetic peptide
AQSVSS

SEQ ID NO: 253 - protein, 6 aa, Artificial Sequence
  Synthetic peptide
AQSVST

SEQ ID NO: 254 - protein, 6 aa, Artificial Sequence
  Synthetic peptide
ASNNNV

SEQ ID NO: 255 - protein, 6 aa, Artificial Sequence
  Synthetic peptide
ASNQNQ

SEQ ID NO: 256 - protein, 6 aa, Artificial Sequence
  Synthetic peptide
ASNQNV

SEQ ID NO: 257 - protein, 6 aa, Artificial Sequence
  Synthetic peptide
ASNQTQ

SEQ ID NO: 258 - protein, 6 aa, Artificial Sequence
  Synthetic peptide
ASNSNV

SEQ ID NO: 259 - protein, 6 aa, Artificial Sequence
  Synthetic peptide
ASNSTV

SEQ ID NO: 260 - protein, 6 aa, Artificial Sequence
  Synthetic peptide
ASNTNS

SEQ ID NO: 261 - protein, 6 aa, Artificial Sequence
  Synthetic peptide
ASNTNV

SEQ ID NO: 262 - protein, 6 aa, Artificial Sequence
  Synthetic peptide
ASNTSV

SEQ ID NO: 263 - protein, 6 aa, Artificial Sequence
  Synthetic peptide
ASNVNG

SEQ ID NO: 264 - protein, 6 aa, Artificial Sequence
  Synthetic peptide
ASNVNQ

SEQ ID NO: 265 - protein, 6 aa, Artificial Sequence
  Synthetic peptide
ASNVNS

SEQ ID NO: 266 - protein, 6 aa, Artificial Sequence
  Synthetic peptide
ASNVNT

SEQ ID NO: 267 - protein, 6 aa, Artificial Sequence
  Synthetic peptide
ASNVTG

SEQ ID NO: 268 - protein, 6 aa, Artificial Sequence
  Synthetic peptide
ASNVTT

SEQ ID NO: 269 - protein, 6 aa, Artificial Sequence
  Synthetic peptide
ASSNSV

SEQ ID NO: 270 - protein, 6 aa, Artificial Sequence
  Synthetic peptide
ASSVSG

SEQ ID NO: 271 - protein, 6 aa, Artificial Sequence
  Synthetic peptide
ASSVSN

SEQ ID NO: 272 - protein, 6 aa, Artificial Sequence
  Synthetic peptide
ATNVNS

SEQ ID NO: 273 - protein, 6 aa, Artificial Sequence
  Synthetic peptide
ATNVTS

SEQ ID NO: 274 - protein, 6 aa, Artificial Sequence
  Synthetic peptide
ATSQSQ

SEQ ID NO: 275 - protein, 6 aa, Artificial Sequence
  Synthetic peptide
ATSQSV

SEQ ID NO: 276 - protein, 6 aa, Artificial Sequence
  Synthetic peptide
ATSTSG

SEQ ID NO: 277 - protein, 6 aa, Artificial Sequence
  Synthetic peptide
ATSTSV

SEQ ID NO: 278 - protein, 6 aa, Artificial Sequence
  Synthetic peptide
ATSVSG

SEQ ID NO: 279 - protein, 6 aa, Artificial Sequence
  Synthetic peptide
ATSVSS

SEQ ID NO: 280 - protein, 6 aa, Artificial Sequence
  Synthetic peptide
AVNQNS

SEQ ID NO: 281 - protein, 6 aa, Artificial Sequence
  Synthetic peptide
AVNQSQ

SEQ ID NO: 282 - protein, 6 aa, Artificial Sequence
  Synthetic peptide
AVNSNG

SEQ ID NO: 283 - protein, 6 aa, Artificial Sequence
  Synthetic peptide
AVNSNS

SEQ ID NO: 284 - protein, 6 aa, Artificial Sequence
  Synthetic peptide
AVNSNT

SEQ ID NO: 285 - protein, 6 aa, Artificial Sequence
  Synthetic peptide
AVNTNS

SEQ ID NO: 286 - protein, 6 aa, Artificial Sequence
  Synthetic peptide
AVSNSG

SEQ ID NO: 287 - protein, 6 aa, Artificial Sequence
  Synthetic peptide
AVSNSS

SEQ ID NO: 288 - protein, 6 aa, Artificial Sequence
  Synthetic peptide
AVSQNQ

SEQ ID NO: 289 - protein, 6 aa, Artificial Sequence
  Synthetic peptide
AVSQSG

SEQ ID NO: 290 - protein, 6 aa, Artificial Sequence
  Synthetic peptide
AVSQSQ

SEQ ID NO: 291 - protein, 6 aa, Artificial Sequence
  Synthetic peptide
AVSQTQ

SEQ ID NO: 292 - protein, 6 aa, Artificial Sequence
  Synthetic peptide
AVSSNQ

SEQ ID NO: 293 - protein, 6 aa, Artificial Sequence
  Synthetic peptide
AVSSNS

SEQ ID NO: 294 - protein, 6 aa, Artificial Sequence
  Synthetic peptide
AVSSSQ

SEQ ID NO: 295 - protein, 6 aa, Artificial Sequence
  Synthetic peptide
AVSTSG

SEQ ID NO: 296 - protein, 6 aa, Artificial Sequence
  Synthetic peptide
GANTVS

SEQ ID NO: 297 - protein, 6 aa, Artificial Sequence
  Synthetic peptide
GAQTSS

SEQ ID NO: 298 - protein, 6 aa, Artificial Sequence
  Synthetic peptide
GASNQS

SEQ ID NO: 299 - protein, 6 aa, Artificial Sequence
  Synthetic peptide
GASQQS

SEQ ID NO: 300 - protein, 6 aa, Artificial Sequence
  Synthetic peptide
GASSQQ

SEQ ID NO: 301 - protein, 6 aa, Artificial Sequence
  Synthetic peptide
GGQVTS

SEQ ID NO: 302 - protein, 6 aa, Artificial Sequence
  Synthetic peptide
GGSNQV

SEQ ID NO: 303 - protein, 6 aa, Artificial Sequence
  Synthetic peptide
GNNVQS

SEQ ID NO: 304 - protein, 6 aa, Artificial Sequence
  Synthetic peptide
GNQVTS

SEQ ID NO: 305 - protein, 6 aa, Artificial Sequence
  Synthetic peptide
GNSNQV

SEQ ID NO: 306 - protein, 6 aa, Artificial Sequence
  Synthetic peptide
GNSQQQ

SEQ ID NO: 307 - protein, 6 aa, Artificial Sequence
  Synthetic peptide
GNSQQS

SEQ ID NO: 308 - protein, 6 aa, Artificial Sequence
  Synthetic peptide
GNSQQV

SEQ ID NO: 309 - protein, 6 aa, Artificial Sequence
  Synthetic peptide
GNSSTV

SEQ ID NO: 310 - protein, 6 aa, Artificial Sequence
  Synthetic peptide
GNSTQS

SEQ ID NO: 311 - protein, 6 aa, Artificial Sequence
  Synthetic peptide
GNSTQV

SEQ ID NO: 312 - protein, 6 aa, Artificial Sequence
  Synthetic peptide
GNSTVS

SEQ ID NO: 313 - protein, 6 aa, Artificial Sequence
  Synthetic peptide
GNSVQS

SEQ ID NO: 314 - protein, 6 aa, Artificial Sequence
  Synthetic peptide
GNSVSS

SEQ ID NO: 315 - protein, 6 aa, Artificial Sequence
  Synthetic peptide
GNSVST

SEQ ID NO: 316 - protein, 6 aa, Artificial Sequence
  Synthetic peptide
GNSVTS

SEQ ID NO: 317 - protein, 6 aa, Artificial Sequence
  Synthetic peptide
GQNTVS

SEQ ID NO: 318 - protein, 6 aa, Artificial Sequence
  Synthetic peptide
GQNVAS

SEQ ID NO: 319 - protein, 6 aa, Artificial Sequence
  Synthetic peptide
GQNVQS

SEQ ID NO: 320 - protein, 6 aa, Artificial Sequence
  Synthetic peptide
GQNVSS

SEQ ID NO: 321 - protein, 6 aa, Artificial Sequence
  Synthetic peptide
GQNVTS

SEQ ID NO: 322 - protein, 6 aa, Artificial Sequence
  Synthetic peptide
GQQQSQ

SEQ ID NO: 323 - protein, 6 aa, Artificial Sequence
  Synthetic peptide
GQQTSS

SEQ ID NO: 324 - protein, 6 aa, Artificial Sequence
  Synthetic peptide
GQQTSV

SEQ ID NO: 325 - protein, 6 aa, Artificial Sequence
  Synthetic peptide
GQQVAS

SEQ ID NO: 326 - protein, 6 aa, Artificial Sequence
  Synthetic peptide
GQQVNS

SEQ ID NO: 327 - protein, 6 aa, Artificial Sequence
  Synthetic peptide
GQQVQS

SEQ ID NO: 328 - protein, 6 aa, Artificial Sequence
  Synthetic peptide
GQQVSG

SEQ ID NO: 329 - protein, 6 aa, Artificial Sequence
  Synthetic peptide
GQQVSQ

SEQ ID NO: 330 - protein, 6 aa, Artificial Sequence
  Synthetic peptide
GQQVSS

SEQ ID NO: 331 - protein, 6 aa, Artificial Sequence
  Synthetic peptide
GQQVST

SEQ ID NO: 332 - protein, 6 aa, Artificial Sequence
  Synthetic peptide
GQQVTS

SEQ ID NO: 333 - protein, 6 aa, Artificial Sequence
  Synthetic peptide
GQSGQV

SEQ ID NO: 334 - protein, 6 aa, Artificial Sequence
  Synthetic peptide
GQSNQA

SEQ ID NO: 335 - protein, 6 aa, Artificial Sequence
  Synthetic peptide
GQSNQS

SEQ ID NO: 336 - protein, 6 aa, Artificial Sequence
  Synthetic peptide
GQSNQV

SEQ ID NO: 337 - protein, 6 aa, Artificial Sequence
  Synthetic peptide
GQSQAQ

SEQ ID NO: 338 - protein, 6 aa, Artificial Sequence
  Synthetic peptide
GQSQQQ

SEQ ID NO: 339 - protein, 6 aa, Artificial Sequence
  Synthetic peptide
GQSQQS

SEQ ID NO: 340 - protein, 6 aa, Artificial Sequence
  Synthetic peptide
GQSQSQ

SEQ ID NO: 341 - protein, 6 aa, Artificial Sequence
  Synthetic peptide
GQSSQQ

SEQ ID NO: 342 - protein, 6 aa, Artificial Sequence
  Synthetic peptide
GQSSQS

SEQ ID NO: 343 - protein, 6 aa, Artificial Sequence
  Synthetic peptide
GQSSQV

SEQ ID NO: 344 - protein, 6 aa, Artificial Sequence
  Synthetic peptide
GQSTQS

SEQ ID NO: 345 - protein, 6 aa, Artificial Sequence
  Synthetic peptide
GQSTQV

SEQ ID NO: 346 - protein, 6 aa, Artificial Sequence
  Synthetic peptide
GQSVAG

SEQ ID NO: 347 - protein, 6 aa, Artificial Sequence
  Synthetic peptide
GQSVAQ

SEQ ID NO: 348 - protein, 6 aa, Artificial Sequence
  Synthetic peptide
GQSVAS

SEQ ID NO: 349 - protein, 6 aa, Artificial Sequence
  Synthetic peptide
GQSVQN

SEQ ID NO: 350 - protein, 6 aa, Artificial Sequence
  Synthetic peptide
GQSVQQ

SEQ ID NO: 351 - protein, 6 aa, Artificial Sequence
  Synthetic peptide
GQSVQS

SEQ ID NO: 352 - protein, 6 aa, Artificial Sequence
  Synthetic peptide
GQSVSG

SEQ ID NO: 353 - protein, 6 aa, Artificial Sequence
  Synthetic peptide
GQSVSN

SEQ ID NO: 354 - protein, 6 aa, Artificial Sequence
  Synthetic peptide
GQSVSQ

SEQ ID NO: 355 - protein, 6 aa, Artificial Sequence
  Synthetic peptide
GQSVSS

SEQ ID NO: 356 - protein, 6 aa, Artificial Sequence
  Synthetic peptide
GQSVST

SEQ ID NO: 357 - protein, 6 aa, Artificial Sequence
  Synthetic peptide
GQSVTS

SEQ ID NO: 358 - protein, 6 aa, Artificial Sequence
  Synthetic peptide
GSNQQV

SEQ ID NO: 359 - protein, 6 aa, Artificial Sequence
  Synthetic peptide
GSNQVQ

SEQ ID NO: 360 - protein, 6 aa, Artificial Sequence
  Synthetic peptide
GSNSTV

SEQ ID NO: 361 - protein, 6 aa, Artificial Sequence
  Synthetic peptide
GSNSVQ

SEQ ID NO: 362 - protein, 6 aa, Artificial Sequence
  Synthetic peptide
GSNSVT

SEQ ID NO: 363 - protein, 6 aa, Artificial Sequence
  Synthetic peptide
GSNTAV

SEQ ID NO: 364 - protein, 6 aa, Artificial Sequence
  Synthetic peptide
GSNTQV

SEQ ID NO: 365 - protein, 6 aa, Artificial Sequence
  Synthetic peptide
GSNTVA

SEQ ID NO: 366 - protein, 6 aa, Artificial Sequence
  Synthetic peptide
GSNTVS

SEQ ID NO: 367 - protein, 6 aa, Artificial Sequence
  Synthetic peptide
GSNVAS

SEQ ID NO: 368 - protein, 6 aa, Artificial Sequence
  Synthetic peptide
GSNVQQ

SEQ ID NO: 369 - protein, 6 aa, Artificial Sequence
  Synthetic peptide
GSNVQS

SEQ ID NO: 370 - protein, 6 aa, Artificial Sequence
  Synthetic peptide
GSNVQT

SEQ ID NO: 371 - protein, 6 aa, Artificial Sequence
  Synthetic peptide
GSNVTS

SEQ ID NO: 372 - protein, 6 aa, Artificial Sequence
  Synthetic peptide
GSQQSV

SEQ ID NO: 373 - protein, 6 aa, Artificial Sequence
  Synthetic peptide
GSQQTQ

SEQ ID NO: 374 - protein, 6 aa, Artificial Sequence
  Synthetic peptide
GSQQTV

SEQ ID NO: 375 - protein, 6 aa, Artificial Sequence
  Synthetic peptide
GSQTSS

SEQ ID NO: 376 - protein, 6 aa, Artificial Sequence
  Synthetic peptide
GSQTSV

SEQ ID NO: 377 - protein, 6 aa, Artificial Sequence
  Synthetic peptide
GSQVAS

SEQ ID NO: 378 - protein, 6 aa, Artificial Sequence
  Synthetic peptide
GSQVNS

SEQ ID NO: 379 - protein, 6 aa, Artificial Sequence
  Synthetic peptide
GSQVQS

SEQ ID NO: 380 - protein, 6 aa, Artificial Sequence
  Synthetic peptide
GSQVSS

SEQ ID NO: 381 - protein, 6 aa, Artificial Sequence
  Synthetic peptide
GSQVST

SEQ ID NO: 382 - protein, 6 aa, Artificial Sequence
  Synthetic peptide
GSQVTG

SEQ ID NO: 383 - protein, 6 aa, Artificial Sequence
  Synthetic peptide
GSQVTS

SEQ ID NO: 384 - protein, 6 aa, Artificial Sequence
  Synthetic peptide
GSSAQS

SEQ ID NO: 385 - protein, 6 aa, Artificial Sequence
  Synthetic peptide
GSSGQV

SEQ ID NO: 386 - protein, 6 aa, Artificial Sequence
  Synthetic peptide
GSSNQA

SEQ ID NO: 387 - protein, 6 aa, Artificial Sequence
  Synthetic peptide
GSSNQT

SEQ ID NO: 388 - protein, 6 aa, Artificial Sequence
  Synthetic peptide
GSSNQV

SEQ ID NO: 389 - protein, 6 aa, Artificial Sequence
  Synthetic peptide
GSSNSV

SEQ ID NO: 390 - protein, 6 aa, Artificial Sequence
  Synthetic peptide
GSSNTV

SEQ ID NO: 391 - protein, 6 aa, Artificial Sequence
  Synthetic peptide
GSSNVT

SEQ ID NO: 392 - protein, 6 aa, Artificial Sequence
  Synthetic peptide
GSSQAQ

SEQ ID NO: 393 - protein, 6 aa, Artificial Sequence
  Synthetic peptide
GSSQQA

SEQ ID NO: 394 - protein, 6 aa, Artificial Sequence
  Synthetic peptide
GSSQQV

SEQ ID NO: 395 - protein, 6 aa, Artificial Sequence
  Synthetic peptide
GSSQSQ

SEQ ID NO: 396 - protein, 6 aa, Artificial Sequence
  Synthetic peptide
GSSSQQ

SEQ ID NO: 397 - protein, 6 aa, Artificial Sequence
  Synthetic peptide
GSSSQS

SEQ ID NO: 398 - protein, 6 aa, Artificial Sequence
  Synthetic peptide
GSSSQV

SEQ ID NO: 399 - protein, 6 aa, Artificial Sequence
  Synthetic peptide
GSSTQA

SEQ ID NO: 400 - protein, 6 aa, Artificial Sequence
  Synthetic peptide
GSSTQG

SEQ ID NO: 401 - protein, 6 aa, Artificial Sequence
  Synthetic peptide
GSSTQT

SEQ ID NO: 402 - protein, 6 aa, Artificial Sequence
  Synthetic peptide
GSSTQV

SEQ ID NO: 403 - protein, 6 aa, Artificial Sequence
  Synthetic peptide
GSSTSV

SEQ ID NO: 404 - protein, 6 aa, Artificial Sequence
  Synthetic peptide
GSSTVN

SEQ ID NO: 405 - protein, 6 aa, Artificial Sequence
  Synthetic peptide
GSSVNS

SEQ ID NO: 406 - protein, 6 aa, Artificial Sequence
  Synthetic peptide
GSSVQN

SEQ ID NO: 407 - protein, 6 aa, Artificial Sequence
  Synthetic peptide
GSSVQT

SEQ ID NO: 408 - protein, 6 aa, Artificial Sequence
  Synthetic peptide
GSSVSG

SEQ ID NO: 409 - protein, 6 aa, Artificial Sequence
  Synthetic peptide
GSSVSS

SEQ ID NO: 410 - protein, 6 aa, Artificial Sequence
  Synthetic peptide
GSSVST

SEQ ID NO: 411 - protein, 6 aa, Artificial Sequence
  Synthetic peptide
GSSVTN

SEQ ID NO: 412 - protein, 6 aa, Artificial Sequence
  Synthetic peptide
GTNSSV

SEQ ID NO: 413 - protein, 6 aa, Artificial Sequence
  Synthetic peptide
GTNSVS

SEQ ID NO: 414 - protein, 6 aa, Artificial Sequence
  Synthetic peptide
GTNTVS

SEQ ID NO: 415 - protein, 6 aa, Artificial Sequence
  Synthetic peptide
GTNVQS

SEQ ID NO: 416 - protein, 6 aa, Artificial Sequence
  Synthetic peptide
GTNVSS

SEQ ID NO: 417 - protein, 6 aa, Artificial Sequence
  Synthetic peptide
GTNVTS

SEQ ID NO: 418 - protein, 6 aa, Artificial Sequence
  Synthetic peptide
GTQQSQ

SEQ ID NO: 419 - protein, 6 aa, Artificial Sequence
  Synthetic peptide
GTQSTS

SEQ ID NO: 420 - protein, 6 aa, Artificial Sequence
  Synthetic peptide
GTQSTV

SEQ ID NO: 421 - protein, 6 aa, Artificial Sequence
  Synthetic peptide
GTQTSA

SEQ ID NO: 422 - protein, 6 aa, Artificial Sequence
  Synthetic peptide
GTQTSV

SEQ ID NO: 423 - protein, 6 aa, Artificial Sequence
  Synthetic peptide
GTQVQS

SEQ ID NO: 424 - protein, 6 aa, Artificial Sequence
  Synthetic peptide
GTQVSG

SEQ ID NO: 425 - protein, 6 aa, Artificial Sequence
  Synthetic peptide
GTQVSN

SEQ ID NO: 426 - protein, 6 aa, Artificial Sequence
  Synthetic peptide
GTQVSS

SEQ ID NO: 427 - protein, 6 aa, Artificial Sequence
  Synthetic peptide
GTQVST

SEQ ID NO: 428 - protein, 6 aa, Artificial Sequence
  Synthetic peptide
GTSAQS

SEQ ID NO: 429 - protein, 6 aa, Artificial Sequence
  Synthetic peptide
GTSGQV

SEQ ID NO: 430 - protein, 6 aa, Artificial Sequence
  Synthetic peptide
GTSNQA

SEQ ID NO: 431 - protein, 6 aa, Artificial Sequence
  Synthetic peptide
GTSNQS

SEQ ID NO: 432 - protein, 6 aa, Artificial Sequence
  Synthetic peptide
GTSNQT

SEQ ID NO: 433 - protein, 6 aa, Artificial Sequence
  Synthetic peptide
GTSNQV

SEQ ID NO: 434 - protein, 6 aa, Artificial Sequence
  Synthetic peptide
GTSNSV

SEQ ID NO: 435 - protein, 6 aa, Artificial Sequence
  Synthetic peptide
GTSNVS

SEQ ID NO: 436 - protein, 6 aa, Artificial Sequence
  Synthetic peptide
GTSQQA

SEQ ID NO: 437 - protein, 6 aa, Artificial Sequence
  Synthetic peptide
GTSQQS

SEQ ID NO: 438 - protein, 6 aa, Artificial Sequence
  Synthetic peptide
GTSQQV

SEQ ID NO: 439 - protein, 6 aa, Artificial Sequence
  Synthetic peptide
GTSQSQ

SEQ ID NO: 440 - protein, 6 aa, Artificial Sequence
  Synthetic peptide
GTSQSV

SEQ ID NO: 441 - protein, 6 aa, Artificial Sequence
  Synthetic peptide
GTSSNV

SEQ ID NO: 442 - protein, 6 aa, Artificial Sequence
  Synthetic peptide
GTSTQA

SEQ ID NO: 443 - protein, 6 aa, Artificial Sequence
  Synthetic peptide
GTSTQS

SEQ ID NO: 444 - protein, 6 aa, Artificial Sequence
  Synthetic peptide
GTSTQT

SEQ ID NO: 445 - protein, 6 aa, Artificial Sequence
  Synthetic peptide
GTSTQV

SEQ ID NO: 446 - protein, 6 aa, Artificial Sequence
  Synthetic peptide
GTSTSV

SEQ ID NO: 447 - protein, 6 aa, Artificial Sequence
  Synthetic peptide
GTSVAG

SEQ ID NO: 448 - protein, 6 aa, Artificial Sequence
  Synthetic peptide
GTSVAS

SEQ ID NO: 449 - protein, 6 aa, Artificial Sequence
  Synthetic peptide
GTSVNS

SEQ ID NO: 450 - protein, 6 aa, Artificial Sequence
  Synthetic peptide
GTSVNT

SEQ ID NO: 451 - protein, 6 aa, Artificial Sequence
  Synthetic peptide
GTSVQG

SEQ ID NO: 452 - protein, 6 aa, Artificial Sequence
  Synthetic peptide
GTSVQN

SEQ ID NO: 453 - protein, 6 aa, Artificial Sequence
  Synthetic peptide
GTSVQQ

SEQ ID NO: 454 - protein, 6 aa, Artificial Sequence
  Synthetic peptide
GTSVQS

SEQ ID NO: 455 - protein, 6 aa, Artificial Sequence
  Synthetic peptide
GTSVQT

SEQ ID NO: 456 - protein, 6 aa, Artificial Sequence
  Synthetic peptide
GTSVSG

SEQ ID NO: 457 - protein, 6 aa, Artificial Sequence
  Synthetic peptide
GTSVSN

SEQ ID NO: 458 - protein, 6 aa, Artificial Sequence
  Synthetic peptide
GTSVSS

SEQ ID NO: 459 - protein, 6 aa, Artificial Sequence
  Synthetic peptide
GTSVST

SEQ ID NO: 460 - protein, 6 aa, Artificial Sequence
  Synthetic peptide
GTSVTS

SEQ ID NO: 461 - protein, 6 aa, Artificial Sequence
  Synthetic peptide
GVNSQS

SEQ ID NO: 462 - protein, 6 aa, Artificial Sequence
  Synthetic peptide
GVNSST

SEQ ID NO: 463 - protein, 6 aa, Artificial Sequence
  Synthetic peptide
GVNSTS

SEQ ID NO: 464 - protein, 6 aa, Artificial Sequence
  Synthetic peptide
GVNTQS

SEQ ID NO: 465 - protein, 6 aa, Artificial Sequence
  Synthetic peptide
GVNTSS

SEQ ID NO: 466 - protein, 6 aa, Artificial Sequence
  Synthetic peptide
GVQNTS

SEQ ID NO: 467 - protein, 6 aa, Artificial Sequence
  Synthetic peptide
GVQQSQ

SEQ ID NO: 468 - protein, 6 aa, Artificial Sequence
  Synthetic peptide
GVQSNQ

SEQ ID NO: 469 - protein, 6 aa, Artificial Sequence
  Synthetic peptide
GVQSNS

SEQ ID NO: 470 - protein, 6 aa, Artificial Sequence
  Synthetic peptide
GVQSSG

SEQ ID NO: 471 - protein, 6 aa, Artificial Sequence
  Synthetic peptide
GVQSSQ

SEQ ID NO: 472 - protein, 6 aa, Artificial Sequence
  Synthetic peptide
GVQSTS

SEQ ID NO: 473 - protein, 6 aa, Artificial Sequence
  Synthetic peptide
GVQSTT

SEQ ID NO: 474 - protein, 6 aa, Artificial Sequence
  Synthetic peptide
GVQTNS

SEQ ID NO: 475 - protein, 6 aa, Artificial Sequence
  Synthetic peptide
GVQTQS

SEQ ID NO: 476 - protein, 6 aa, Artificial Sequence
  Synthetic peptide
GVQTSG

SEQ ID NO: 477 - protein, 6 aa, Artificial Sequence
  Synthetic peptide
GVQTSS

SEQ ID NO: 478 - protein, 6 aa, Artificial Sequence
  Synthetic peptide
GVSGQG

SEQ ID NO: 479 - protein, 6 aa, Artificial Sequence
  Synthetic peptide
GVSGQT

SEQ ID NO: 480 - protein, 6 aa, Artificial Sequence
  Synthetic peptide
GVSNAS

SEQ ID NO: 481 - protein, 6 aa, Artificial Sequence
  Synthetic peptide
GVSNNV

SEQ ID NO: 482 - protein, 6 aa, Artificial Sequence
  Synthetic peptide
GVSNQG

SEQ ID NO: 483 - protein, 6 aa, Artificial Sequence
  Synthetic peptide
GVSNQN

SEQ ID NO: 484 - protein, 6 aa, Artificial Sequence
  Synthetic peptide
GVSNQQ

SEQ ID NO: 485 - protein, 6 aa, Artificial Sequence
  Synthetic peptide
GVSNQS

SEQ ID NO: 486 - protein, 6 aa, Artificial Sequence
  Synthetic peptide
GVSNQT

SEQ ID NO: 487 - protein, 6 aa, Artificial Sequence
  Synthetic peptide
GVSNSS

SEQ ID NO: 488 - protein, 6 aa, Artificial Sequence
  Synthetic peptide
GVSNST

SEQ ID NO: 489 - protein, 6 aa, Artificial Sequence
  Synthetic peptide
GVSNTS

SEQ ID NO: 490 - protein, 6 aa, Artificial Sequence
  Synthetic peptide
GVSQAQ

SEQ ID NO: 491 - protein, 6 aa, Artificial Sequence
  Synthetic peptide
GVSQNQ

SEQ ID NO: 492 - protein, 6 aa, Artificial Sequence
  Synthetic peptide
GVSQNS

SEQ ID NO: 493 - protein, 6 aa, Artificial Sequence
  Synthetic peptide
GVSQQG

SEQ ID NO: 494 - protein, 6 aa, Artificial Sequence
  Synthetic peptide
GVSQQS

SEQ ID NO: 495 - protein, 6 aa, Artificial Sequence
  Synthetic peptide
GVSQQT

SEQ ID NO: 496 - protein, 6 aa, Artificial Sequence
  Synthetic peptide
GVSQSG

SEQ ID NO: 497 - protein, 6 aa, Artificial Sequence
  Synthetic peptide
GVSQSQ

SEQ ID NO: 498 - protein, 6 aa, Artificial Sequence
  Synthetic peptide
GVSQSS

SEQ ID NO: 499 - protein, 6 aa, Artificial Sequence
  Synthetic peptide
GVSSAQ

SEQ ID NO: 500 - protein, 6 aa, Artificial Sequence
  Synthetic peptide
GVSSAS

SEQ ID NO: 501 - protein, 6 aa, Artificial Sequence
  Synthetic peptide
GVSSGQ

SEQ ID NO: 502 - protein, 6 aa, Artificial Sequence
  Synthetic peptide
GVSSNG

SEQ ID NO: 503 - protein, 6 aa, Artificial Sequence
  Synthetic peptide
GVSSNQ

SEQ ID NO: 504 - protein, 6 aa, Artificial Sequence
  Synthetic peptide
GVSSNS

SEQ ID NO: 505 - protein, 6 aa, Artificial Sequence
  Synthetic peptide
GVSSNT

SEQ ID NO: 506 - protein, 6 aa, Artificial Sequence
  Synthetic peptide
GVSSQG

SEQ ID NO: 507 - protein, 6 aa, Artificial Sequence
  Synthetic peptide
GVSSQN

SEQ ID NO: 508 - protein, 6 aa, Artificial Sequence
  Synthetic peptide
GVSSQQ

SEQ ID NO: 509 - protein, 6 aa, Artificial Sequence
  Synthetic peptide
GVSSSG

SEQ ID NO: 510 - protein, 6 aa, Artificial Sequence
  Synthetic peptide
GVSSSQ

SEQ ID NO: 511 - protein, 6 aa, Artificial Sequence
  Synthetic peptide
GVSSSS

SEQ ID NO: 512 - protein, 6 aa, Artificial Sequence
  Synthetic peptide
GVSSTN

SEQ ID NO: 513 - protein, 6 aa, Artificial Sequence
  Synthetic peptide
GVSTAS

SEQ ID NO: 514 - protein, 6 aa, Artificial Sequence
  Synthetic peptide
GVSTNS

SEQ ID NO: 515 - protein, 6 aa, Artificial Sequence
  Synthetic peptide
GVSTQG

SEQ ID NO: 516 - protein, 6 aa, Artificial Sequence
  Synthetic peptide
GVSTQN

SEQ ID NO: 517 - protein, 6 aa, Artificial Sequence
  Synthetic peptide
GVSTQQ

SEQ ID NO: 518 - protein, 6 aa, Artificial Sequence
  Synthetic peptide
GVSTQS

SEQ ID NO: 519 - protein, 6 aa, Artificial Sequence
  Synthetic peptide
GVSTQT

SEQ ID NO: 520 - protein, 6 aa, Artificial Sequence
  Synthetic peptide
GVSTSN

SEQ ID NO: 521 - protein, 6 aa, Artificial Sequence
  Synthetic peptide
GVSTSQ

SEQ ID NO: 522 - protein, 6 aa, Artificial Sequence
  Synthetic peptide
GVSTSS

SEQ ID NO: 523 - protein, 6 aa, Artificial Sequence
  Synthetic peptide
GVSTST

SEQ ID NO: 524 - protein, 6 aa, Artificial Sequence
  Synthetic peptide
GVTNSS

SEQ ID NO: 525 - protein, 6 aa, Artificial Sequence
  Synthetic peptide
GVTSNS

SEQ ID NO: 526 - protein, 6 aa, Artificial Sequence
  Synthetic peptide
GVTSSN

SEQ ID NO: 527 - protein, 6 aa, Artificial Sequence
  Synthetic peptide
NGSTSV

SEQ ID NO: 528 - protein, 6 aa, Artificial Sequence
  Synthetic peptide
NGSVTS

SEQ ID NO: 529 - protein, 6 aa, Artificial Sequence
  Synthetic peptide
NNSVSS

SEQ ID NO: 530 - protein, 6 aa, Artificial Sequence
  Synthetic peptide
NQSQSQ

SEQ ID NO: 531 - protein, 6 aa, Artificial Sequence
  Synthetic peptide
NQSVSN

SEQ ID NO: 532 - protein, 6 aa, Artificial Sequence
  Synthetic peptide
NQSVSQ

SEQ ID NO: 533 - protein, 6 aa, Artificial Sequence
  Synthetic peptide
NQSVSS

SEQ ID NO: 534 - protein, 6 aa, Artificial Sequence
  Synthetic peptide
NSSNSV

SEQ ID NO: 535 - protein, 6 aa, Artificial Sequence
  Synthetic peptide
NSSQSQ

SEQ ID NO: 536 - protein, 6 aa, Artificial Sequence
  Synthetic peptide
NSSTVG

SEQ ID NO: 537 - protein, 6 aa, Artificial Sequence
  Synthetic peptide
NSSVSG

SEQ ID NO: 538 - protein, 6 aa, Artificial Sequence
  Synthetic peptide
NSSVSN

SEQ ID NO: 539 - protein, 6 aa, Artificial Sequence
  Synthetic peptide
NSSVSS

SEQ ID NO: 540 - protein, 6 aa, Artificial Sequence
  Synthetic peptide
NSSVTG

SEQ ID NO: 541 - protein, 6 aa, Artificial Sequence
  Synthetic peptide
NTNVNS

SEQ ID NO: 542 - protein, 6 aa, Artificial Sequence
  Synthetic peptide
NTQVSS

SEQ ID NO: 543 - protein, 6 aa, Artificial Sequence
  Synthetic peptide
NTSGSV

SEQ ID NO: 544 - protein, 6 aa, Artificial Sequence
  Synthetic peptide
NTSQSQ

SEQ ID NO: 545 - protein, 6 aa, Artificial Sequence
  Synthetic peptide
NTSTSS

SEQ ID NO: 546 - protein, 6 aa, Artificial Sequence
  Synthetic peptide
NTSTSV

SEQ ID NO: 547 - protein, 6 aa, Artificial Sequence
  Synthetic peptide
NTSVSS

SEQ ID NO: 548 - protein, 6 aa, Artificial Sequence
  Synthetic peptide
NVSGST

SEQ ID NO: 549 - protein, 6 aa, Artificial Sequence
  Synthetic peptide
NVSNSG

SEQ ID NO: 550 - protein, 6 aa, Artificial Sequence
  Synthetic peptide
NVSQSG

SEQ ID NO: 551 - protein, 6 aa, Artificial Sequence
  Synthetic peptide
NVSQSQ

SEQ ID NO: 552 - protein, 6 aa, Artificial Sequence
  Synthetic peptide
NVSQSS

SEQ ID NO: 553 - protein, 6 aa, Artificial Sequence
  Synthetic peptide
NVSSSG

SEQ ID NO: 554 - protein, 6 aa, Artificial Sequence
  Synthetic peptide
NVSSSS

SEQ ID NO: 555 - protein, 6 aa, Artificial Sequence
  Synthetic peptide
NVSSTG

SEQ ID NO: 556 - protein, 6 aa, Artificial Sequence
  Synthetic peptide
NVSTSN

SEQ ID NO: 557 - protein, 6 aa, Artificial Sequence
  Synthetic peptide
NVSTSS

SEQ ID NO: 558 - protein, 6 aa, Artificial Sequence
  Synthetic peptide
NVSTST

SEQ ID NO: 559 - protein, 6 aa, Artificial Sequence
  Synthetic peptide
QNSTSV

SEQ ID NO: 560 - protein, 6 aa, Artificial Sequence
  Synthetic peptide
QNSVSG

SEQ ID NO: 561 - protein, 6 aa, Artificial Sequence
  Synthetic peptide
QQSQSQ

SEQ ID NO: 562 - protein, 6 aa, Artificial Sequence
  Synthetic peptide
QQSVAG

SEQ ID NO: 563 - protein, 6 aa, Artificial Sequence
  Synthetic peptide
QQSVAQ

SEQ ID NO: 564 - protein, 6 aa, Artificial Sequence
  Synthetic peptide
QQSVAS

SEQ ID NO: 565 - protein, 6 aa, Artificial Sequence
  Synthetic peptide
QQSVAT

SEQ ID NO: 566 - protein, 6 aa, Artificial Sequence
  Synthetic peptide
QQSVSG

SEQ ID NO: 567 - protein, 6 aa, Artificial Sequence
  Synthetic peptide
QQSVSN

SEQ ID NO: 568 - protein, 6 aa, Artificial Sequence
  Synthetic peptide
QQSVSS

SEQ ID NO: 569 - protein, 6 aa, Artificial Sequence
  Synthetic peptide
QSNQVQ

SEQ ID NO: 570 - protein, 6 aa, Artificial Sequence
  Synthetic peptide
QSNTAV

SEQ ID NO: 571 - protein, 6 aa, Artificial Sequence
  Synthetic peptide
QSNTNV

SEQ ID NO: 572 - protein, 6 aa, Artificial Sequence
  Synthetic peptide
QSSNSV

SEQ ID NO: 573 - protein, 6 aa, Artificial Sequence
  Synthetic peptide
QSSQAQ

SEQ ID NO: 574 - protein, 6 aa, Artificial Sequence
  Synthetic peptide
QSSQSQ

SEQ ID NO: 575 - protein, 6 aa, Artificial Sequence
  Synthetic peptide
QSSQSV

SEQ ID NO: 576 - protein, 6 aa, Artificial Sequence
  Synthetic peptide
QSSTSV

SEQ ID NO: 577 - protein, 6 aa, Artificial Sequence
  Synthetic peptide
QSSVSG

SEQ ID NO: 578 - protein, 6 aa, Artificial Sequence
  Synthetic peptide
QSSVSN

SEQ ID NO: 579 - protein, 6 aa, Artificial Sequence
  Synthetic peptide
QSSVSQ

SEQ ID NO: 580 - protein, 6 aa, Artificial Sequence
  Synthetic peptide
QSSVSS

SEQ ID NO: 581 - protein, 6 aa, Artificial Sequence
  Synthetic peptide
QSSVTG

SEQ ID NO: 582 - protein, 6 aa, Artificial Sequence
  Synthetic peptide
QSSVTS

SEQ ID NO: 583 - protein, 6 aa, Artificial Sequence
  Synthetic peptide
QTSQSQ

SEQ ID NO: 584 - protein, 6 aa, Artificial Sequence
  Synthetic peptide
QTSSSQ

SEQ ID NO: 585 - protein, 6 aa, Artificial Sequence
  Synthetic peptide
QTSSSV

SEQ ID NO: 586 - protein, 6 aa, Artificial Sequence
  Synthetic peptide
QTSVAG

SEQ ID NO: 587 - protein, 6 aa, Artificial Sequence
  Synthetic peptide
QTSVAS

SEQ ID NO: 588 - protein, 6 aa, Artificial Sequence
  Synthetic peptide
QTSVSG

SEQ ID NO: 589 - protein, 6 aa, Artificial Sequence
  Synthetic peptide
QTSVSN

SEQ ID NO: 590 - protein, 6 aa, Artificial Sequence
  Synthetic peptide
QTSVSS

SEQ ID NO: 591 - protein, 6 aa, Artificial Sequence
  Synthetic peptide
QTSVST

SEQ ID NO: 592 - protein, 6 aa, Artificial Sequence
  Synthetic peptide
QTSVTS

SEQ ID NO: 593 - protein, 6 aa, Artificial Sequence
  Synthetic peptide
QVSNSG

SEQ ID NO: 594 - protein, 6 aa, Artificial Sequence
  Synthetic peptide
QVSNSS

SEQ ID NO: 595 - protein, 6 aa, Artificial Sequence
  Synthetic peptide
QVSNST

SEQ ID NO: 596 - protein, 6 aa, Artificial Sequence
  Synthetic peptide
QVSQAQ

SEQ ID NO: 597 - protein, 6 aa, Artificial Sequence
  Synthetic peptide
QVSQTG

SEQ ID NO: 598 - protein, 6 aa, Artificial Sequence
  Synthetic peptide
QVSQTQ

SEQ ID NO: 599 - protein, 6 aa, Artificial Sequence
  Synthetic peptide
QVSSAQ

SEQ ID NO: 600 - protein, 6 aa, Artificial Sequence
  Synthetic peptide
QVSSSG

SEQ ID NO: 601 - protein, 6 aa, Artificial Sequence
  Synthetic peptide
QVSSSQ

SEQ ID NO: 602 - protein, 6 aa, Artificial Sequence
  Synthetic peptide
QVSSSS

SEQ ID NO: 603 - protein, 6 aa, Artificial Sequence
  Synthetic peptide
QVSSTQ

SEQ ID NO: 604 - protein, 6 aa, Artificial Sequence
  Synthetic peptide
QVSTSG

SEQ ID NO: 605 - protein, 6 aa, Artificial Sequence
  Synthetic peptide
QVSTSN

SEQ ID NO: 606 - protein, 6 aa, Artificial Sequence
  Synthetic peptide
QVSTSS

SEQ ID NO: 607 - protein, 6 aa, Artificial Sequence
  Synthetic peptide
QVSTST

SEQ ID NO: 608 - protein, 6 aa, Artificial Sequence
  Synthetic peptide
SAQQSQ

SEQ ID NO: 609 - protein, 6 aa, Artificial Sequence
  Synthetic peptide
SAQQTQ

SEQ ID NO: 610 - protein, 6 aa, Artificial Sequence
  Synthetic peptide
SASQQQ

SEQ ID NO: 611 - protein, 6 aa, Artificial Sequence
  Synthetic peptide
SASQSQ

SEQ ID NO: 612 - protein, 6 aa, Artificial Sequence
  Synthetic peptide
SGNSTV

SEQ ID NO: 613 - protein, 6 aa, Artificial Sequence
  Synthetic peptide
SGNTSV

SEQ ID NO: 614 - protein, 6 aa, Artificial Sequence
  Synthetic peptide
SGNVST

SEQ ID NO: 615 - protein, 6 aa, Artificial Sequence
  Synthetic peptide
SGNVTS

SEQ ID NO: 616 - protein, 6 aa, Artificial Sequence
  Synthetic peptide
SGQQTQ

SEQ ID NO: 617 - protein, 6 aa, Artificial Sequence
  Synthetic peptide
SGQQTV

SEQ ID NO: 618 - protein, 6 aa, Artificial Sequence
  Synthetic peptide
SGQTSV

SEQ ID NO: 619 - protein, 6 aa, Artificial Sequence
  Synthetic peptide
SGQVSS

SEQ ID NO: 620 - protein, 6 aa, Artificial Sequence
  Synthetic peptide
SGQVTG

SEQ ID NO: 621 - protein, 6 aa, Artificial Sequence
  Synthetic peptide
SGQVTQ

SEQ ID NO: 622 - protein, 6 aa, Artificial Sequence
  Synthetic peptide
SGQVTT

SEQ ID NO: 623 - protein, 6 aa, Artificial Sequence
  Synthetic peptide
SGSNTV

SEQ ID NO: 624 - protein, 6 aa, Artificial Sequence
  Synthetic peptide
SGSTNV

SEQ ID NO: 625 - protein, 6 aa, Artificial Sequence
  Synthetic peptide
SGSTQV

SEQ ID NO: 626 - protein, 6 aa, Artificial Sequence
  Synthetic peptide
SGSVAS

SEQ ID NO: 627 - protein, 6 aa, Artificial Sequence
  Synthetic peptide
SGSVNT

SEQ ID NO: 628 - protein, 6 aa, Artificial Sequence
  Synthetic peptide
SGSVQG

SEQ ID NO: 629 - protein, 6 aa, Artificial Sequence
  Synthetic peptide
SGSVQS

SEQ ID NO: 630 - protein, 6 aa, Artificial Sequence
  Synthetic peptide
SNNQTV

SEQ ID NO: 631 - protein, 6 aa, Artificial Sequence
  Synthetic peptide
SNNTAV

SEQ ID NO: 632 - protein, 6 aa, Artificial Sequence
  Synthetic peptide
SNNTNV

SEQ ID NO: 633 - protein, 6 aa, Artificial Sequence
  Synthetic peptide
SNNTQV

SEQ ID NO: 634 - protein, 6 aa, Artificial Sequence
  Synthetic peptide
SNQQSQ

SEQ ID NO: 635 - protein, 6 aa, Artificial Sequence
  Synthetic peptide
SNQQSV

SEQ ID NO: 636 - protein, 6 aa, Artificial Sequence
  Synthetic peptide
SNQQTQ

SEQ ID NO: 637 - protein, 6 aa, Artificial Sequence
  Synthetic peptide
SNQQTV

SEQ ID NO: 638 - protein, 6 aa, Artificial Sequence
  Synthetic peptide
SNQSTV

SEQ ID NO: 639 - protein, 6 aa, Artificial Sequence
  Synthetic peptide
SNQTSV

SEQ ID NO: 640 - protein, 6 aa, Artificial Sequence
  Synthetic peptide
SNQVSG

SEQ ID NO: 641 - protein, 6 aa, Artificial Sequence
  Synthetic peptide
SNQVSQ

SEQ ID NO: 642 - protein, 6 aa, Artificial Sequence
  Synthetic peptide
SNQVSS

SEQ ID NO: 643 - protein, 6 aa, Artificial Sequence
  Synthetic peptide
SNQVST

SEQ ID NO: 644 - protein, 6 aa, Artificial Sequence
  Synthetic peptide
SNQVTG

SEQ ID NO: 645 - protein, 6 aa, Artificial Sequence
  Synthetic peptide
SNQVTQ

SEQ ID NO: 646 - protein, 6 aa, Artificial Sequence
  Synthetic peptide
SNQVTS

SEQ ID NO: 647 - protein, 6 aa, Artificial Sequence
  Synthetic peptide
SNSGAV

SEQ ID NO: 648 - protein, 6 aa, Artificial Sequence
  Synthetic peptide
SNSNAV

SEQ ID NO: 649 - protein, 6 aa, Artificial Sequence
  Synthetic peptide
SNSNGV

SEQ ID NO: 650 - protein, 6 aa, Artificial Sequence
  Synthetic peptide
SNSNQS

SEQ ID NO: 651 - protein, 6 aa, Artificial Sequence
  Synthetic peptide
SNSQAG

SEQ ID NO: 652 - protein, 6 aa, Artificial Sequence
  Synthetic peptide
SNSQAQ

SEQ ID NO: 653 - protein, 6 aa, Artificial Sequence
  Synthetic peptide
SNSQAS

SEQ ID NO: 654 - protein, 6 aa, Artificial Sequence
  Synthetic peptide
SNSQAT

SEQ ID NO: 655 - protein, 6 aa, Artificial Sequence
  Synthetic peptide
SNSQAV

SEQ ID NO: 656 - protein, 6 aa, Artificial Sequence
  Synthetic peptide
SNSQGQ

SEQ ID NO: 657 - protein, 6 aa, Artificial Sequence
  Synthetic peptide
SNSQGV

SEQ ID NO: 658 - protein, 6 aa, Artificial Sequence
  Synthetic peptide
SNSQQA

SEQ ID NO: 659 - protein, 6 aa, Artificial Sequence
  Synthetic peptide
SNSQQG

SEQ ID NO: 660 - protein, 6 aa, Artificial Sequence
  Synthetic peptide
SNSQQQ

SEQ ID NO: 661 - protein, 6 aa, Artificial Sequence
  Synthetic peptide
SNSQQS

SEQ ID NO: 662 - protein, 6 aa, Artificial Sequence
  Synthetic peptide
SNSQQT

SEQ ID NO: 663 - protein, 6 aa, Artificial Sequence
  Synthetic peptide
SNSQQV

SEQ ID NO: 664 - protein, 6 aa, Artificial Sequence
  Synthetic peptide
SNSQSG

SEQ ID NO: 665 - protein, 6 aa, Artificial Sequence
  Synthetic peptide
SNSQST

SEQ ID NO: 666 - protein, 6 aa, Artificial Sequence
  Synthetic peptide
SNSQSV

SEQ ID NO: 667 - protein, 6 aa, Artificial Sequence
  Synthetic peptide
SNSSAV

SEQ ID NO: 668 - protein, 6 aa, Artificial Sequence
  Synthetic peptide
SNSSGV

SEQ ID NO: 669 - protein, 6 aa, Artificial Sequence
  Synthetic peptide
SNSSQG

SEQ ID NO: 670 - protein, 6 aa, Artificial Sequence
  Synthetic peptide
SNSSQQ

SEQ ID NO: 671 - protein, 6 aa, Artificial Sequence
  Synthetic peptide
SNSSQT

SEQ ID NO: 672 - protein, 6 aa, Artificial Sequence
  Synthetic peptide
SNSSQV

SEQ ID NO: 673 - protein, 6 aa, Artificial Sequence
  Synthetic peptide
SNSSSQ

SEQ ID NO: 674 - protein, 6 aa, Artificial Sequence
  Synthetic peptide
SNSSSV

SEQ ID NO: 675 - protein, 6 aa, Artificial Sequence
  Synthetic peptide
SNSTAG

SEQ ID NO: 676 - protein, 6 aa, Artificial Sequence
  Synthetic peptide
SNSTGS

SEQ ID NO: 677 - protein, 6 aa, Artificial Sequence
  Synthetic peptide
SNSTNV

SEQ ID NO: 678 - protein, 6 aa, Artificial Sequence
  Synthetic peptide
SNSTQA

SEQ ID NO: 679 - protein, 6 aa, Artificial Sequence
  Synthetic peptide
SNSTQQ

SEQ ID NO: 680 - protein, 6 aa, Artificial Sequence
  Synthetic peptide
SNSTQS

SEQ ID NO: 681 - protein, 6 aa, Artificial Sequence
  Synthetic peptide
SNSTQV

SEQ ID NO: 682 - protein, 6 aa, Artificial Sequence
  Synthetic peptide
SNSTSA

SEQ ID NO: 683 - protein, 6 aa, Artificial Sequence
  Synthetic peptide
SNSTSV

SEQ ID NO: 684 - protein, 6 aa, Artificial Sequence
  Synthetic peptide
SNSTVG

SEQ ID NO: 685 - protein, 6 aa, Artificial Sequence
  Synthetic peptide
SNSVAQ

SEQ ID NO: 686 - protein, 6 aa, Artificial Sequence
  Synthetic peptide
SNSVAS

SEQ ID NO: 687 - protein, 6 aa, Artificial Sequence
  Synthetic peptide
SNSVAT

SEQ ID NO: 688 - protein, 6 aa, Artificial Sequence
  Synthetic peptide
SNSVGQ

SEQ ID NO: 689 - protein, 6 aa, Artificial Sequence
  Synthetic peptide
SNSVGS

SEQ ID NO: 690 - protein, 6 aa, Artificial Sequence
  Synthetic peptide
SNSVGT

SEQ ID NO: 691 - protein, 6 aa, Artificial Sequence
  Synthetic peptide
SNSVNS

SEQ ID NO: 692 - protein, 6 aa, Artificial Sequence
  Synthetic peptide
SNSVNT

SEQ ID NO: 693 - protein, 6 aa, Artificial Sequence
  Synthetic peptide
SNSVQQ

SEQ ID NO: 694 - protein, 6 aa, Artificial Sequence
  Synthetic peptide
SNSVQS

SEQ ID NO: 695 - protein, 6 aa, Artificial Sequence
  Synthetic peptide
SNSVSG

SEQ ID NO: 696 - protein, 6 aa, Artificial Sequence
  Synthetic peptide
SNSVSQ

SEQ ID NO: 697 - protein, 6 aa, Artificial Sequence
  Synthetic peptide
SNSVSS

SEQ ID NO: 698 - protein, 6 aa, Artificial Sequence
  Synthetic peptide
SNSVST

SEQ ID NO: 699 - protein, 6 aa, Artificial Sequence
  Synthetic peptide
SNSVTG

SEQ ID NO: 700 - protein, 6 aa, Artificial Sequence
  Synthetic peptide
SQNVAS

SEQ ID NO: 701 - protein, 6 aa, Artificial Sequence
  Synthetic peptide
SQNVNS

SEQ ID NO: 702 - protein, 6 aa, Artificial Sequence
  Synthetic peptide
SQQQSQ

SEQ ID NO: 703 - protein, 6 aa, Artificial Sequence
  Synthetic peptide
SQQQSV

SEQ ID NO: 704 - protein, 6 aa, Artificial Sequence
  Synthetic peptide
SQQQTQ

SEQ ID NO: 705 - protein, 6 aa, Artificial Sequence
  Synthetic peptide
SQQQTV

SEQ ID NO: 706 - protein, 6 aa, Artificial Sequence
  Synthetic peptide
SQQSTV

SEQ ID NO: 707 - protein, 6 aa, Artificial Sequence
  Synthetic peptide
SQQVSG

SEQ ID NO: 708 - protein, 6 aa, Artificial Sequence
  Synthetic peptide
SQQVSN

SEQ ID NO: 709 - protein, 6 aa, Artificial Sequence
  Synthetic peptide
SQQVSS

SEQ ID NO: 710 - protein, 6 aa, Artificial Sequence
  Synthetic peptide
SQQVST

SEQ ID NO: 711 - protein, 6 aa, Artificial Sequence
  Synthetic peptide
SQQVTG

SEQ ID NO: 712 - protein, 6 aa, Artificial Sequence
  Synthetic peptide
SQQVTN

SEQ ID NO: 713 - protein, 6 aa, Artificial Sequence
  Synthetic peptide
SQQVTS

SEQ ID NO: 714 - protein, 6 aa, Artificial Sequence
  Synthetic peptide
SQQVTT

SEQ ID NO: 715 - protein, 6 aa, Artificial Sequence
  Synthetic peptide
SQSNAS

SEQ ID NO: 716 - protein, 6 aa, Artificial Sequence
  Synthetic peptide
SQSNAV

SEQ ID NO: 717 - protein, 6 aa, Artificial Sequence
  Synthetic peptide
SQSNGV

SEQ ID NO: 718 - protein, 6 aa, Artificial Sequence
  Synthetic peptide
SQSNQT

SEQ ID NO: 719 - protein, 6 aa, Artificial Sequence
  Synthetic peptide
SQSNQV

SEQ ID NO: 720 - protein, 6 aa, Artificial Sequence
  Synthetic peptide
SQSNSV

SEQ ID NO: 721 - protein, 6 aa, Artificial Sequence
  Synthetic peptide
SQSQAQ

SEQ ID NO: 722 - protein, 6 aa, Artificial Sequence
  Synthetic peptide
SQSQAS

SEQ ID NO: 723 - protein, 6 aa, Artificial Sequence
  Synthetic peptide
SQSQAV

SEQ ID NO: 724 - protein, 6 aa, Artificial Sequence
  Synthetic peptide
SQSQQQ

SEQ ID NO: 725 - protein, 6 aa, Artificial Sequence
  Synthetic peptide
SQSQQV

SEQ ID NO: 726 - protein, 6 aa, Artificial Sequence
  Synthetic peptide
SQSQSQ

SEQ ID NO: 727 - protein, 6 aa, Artificial Sequence
  Synthetic peptide
SQSSAV

SEQ ID NO: 728 - protein, 6 aa, Artificial Sequence
  Synthetic peptide
SQSSGV

SEQ ID NO: 729 - protein, 6 aa, Artificial Sequence
  Synthetic peptide
SQSSQG

SEQ ID NO: 730 - protein, 6 aa, Artificial Sequence
  Synthetic peptide
SQSSQV

SEQ ID NO: 731 - protein, 6 aa, Artificial Sequence
  Synthetic peptide
SQSSSQ

SEQ ID NO: 732 - protein, 6 aa, Artificial Sequence
  Synthetic peptide
SQSSSV

SEQ ID NO: 733 - protein, 6 aa, Artificial Sequence
  Synthetic peptide
SQSTAV

SEQ ID NO: 734 - protein, 6 aa, Artificial Sequence
  Synthetic peptide
SQSTQS

SEQ ID NO: 735 - protein, 6 aa, Artificial Sequence
  Synthetic peptide
SQSTSG

SEQ ID NO: 736 - protein, 6 aa, Artificial Sequence
  Synthetic peptide
SQSTSV

SEQ ID NO: 737 - protein, 6 aa, Artificial Sequence
  Synthetic peptide
SQSVAG

SEQ ID NO: 738 - protein, 6 aa, Artificial Sequence
  Synthetic peptide
SQSVAN

SEQ ID NO: 739 - protein, 6 aa, Artificial Sequence
  Synthetic peptide
SQSVAQ

SEQ ID NO: 740 - protein, 6 aa, Artificial Sequence
  Synthetic peptide
SQSVAS

SEQ ID NO: 741 - protein, 6 aa, Artificial Sequence
  Synthetic peptide
SQSVAT

SEQ ID NO: 742 - protein, 6 aa, Artificial Sequence
  Synthetic peptide
SQSVGG

SEQ ID NO: 743 - protein, 6 aa, Artificial Sequence
  Synthetic peptide
SQSVGN

SEQ ID NO: 744 - protein, 6 aa, Artificial Sequence
  Synthetic peptide
SQSVGQ

SEQ ID NO: 745 - protein, 6 aa, Artificial Sequence
  Synthetic peptide
SQSVGS

SEQ ID NO: 746 - protein, 6 aa, Artificial Sequence
  Synthetic peptide
SQSVGT

SEQ ID NO: 747 - protein, 6 aa, Artificial Sequence
  Synthetic peptide
SQSVNG

SEQ ID NO: 748 - protein, 6 aa, Artificial Sequence
  Synthetic peptide
SQSVNS

SEQ ID NO: 749 - protein, 6 aa, Artificial Sequence
  Synthetic peptide
SQSVQG

SEQ ID NO: 750 - protein, 6 aa, Artificial Sequence
  Synthetic peptide
SQSVQN

SEQ ID NO: 751 - protein, 6 aa, Artificial Sequence
  Synthetic peptide
SQSVQQ

SEQ ID NO: 752 - protein, 6 aa, Artificial Sequence
  Synthetic peptide
SQSVQS

SEQ ID NO: 753 - protein, 6 aa, Artificial Sequence
  Synthetic peptide
SQSVQT

SEQ ID NO: 754 - protein, 6 aa, Artificial Sequence
  Synthetic peptide
SQSVSG

SEQ ID NO: 755 - protein, 6 aa, Artificial Sequence
  Synthetic peptide
SQSVSN

SEQ ID NO: 756 - protein, 6 aa, Artificial Sequence
  Synthetic peptide
SQSVSS

SEQ ID NO: 757 - protein, 6 aa, Artificial Sequence
  Synthetic peptide
SQSVST

SEQ ID NO: 758 - protein, 6 aa, Artificial Sequence
  Synthetic peptide
SSNGTV

SEQ ID NO: 759 - protein, 6 aa, Artificial Sequence
  Synthetic peptide
SSNQNQ

SEQ ID NO: 760 - protein, 6 aa, Artificial Sequence
  Synthetic peptide
SSNQNV

SEQ ID NO: 761 - protein, 6 aa, Artificial Sequence
  Synthetic peptide
SSNQTQ

SEQ ID NO: 762 - protein, 6 aa, Artificial Sequence
  Synthetic peptide
SSNQTV

SEQ ID NO: 763 - protein, 6 aa, Artificial Sequence
  Synthetic peptide
SSNSTV

SEQ ID NO: 764 - protein, 6 aa, Artificial Sequence
  Synthetic peptide
SSNTNV

SEQ ID NO: 765 - protein, 6 aa, Artificial Sequence
  Synthetic peptide
SSNTQV

SEQ ID NO: 766 - protein, 6 aa, Artificial Sequence
  Synthetic peptide
SSNTVG

SEQ ID NO: 767 - protein, 6 aa, Artificial Sequence
  Synthetic peptide
SSNVAQ

SEQ ID NO: 768 - protein, 6 aa, Artificial Sequence
  Synthetic peptide
SSNVAS

SEQ ID NO: 769 - protein, 6 aa, Artificial Sequence
  Synthetic peptide
SSNVGT

SEQ ID NO: 770 - protein, 6 aa, Artificial Sequence
  Synthetic peptide
SSNVNG

SEQ ID NO: 771 - protein, 6 aa, Artificial Sequence
  Synthetic peptide
SSNVNQ

SEQ ID NO: 772 - protein, 6 aa, Artificial Sequence
  Synthetic peptide
SSNVNS

SEQ ID NO: 773 - protein, 6 aa, Artificial Sequence
  Synthetic peptide
SSNVNT

SEQ ID NO: 774 - protein, 6 aa, Artificial Sequence
  Synthetic peptide
SSNVQS

SEQ ID NO: 775 - protein, 6 aa, Artificial Sequence
  Synthetic peptide
SSNVQT

SEQ ID NO: 776 - protein, 6 aa, Artificial Sequence
  Synthetic peptide
SSNVTG

SEQ ID NO: 777 - protein, 6 aa, Artificial Sequence
  Synthetic peptide
SSNVTQ

SEQ ID NO: 778 - protein, 6 aa, Artificial Sequence
  Synthetic peptide
SSNVTS

SEQ ID NO: 779 - protein, 6 aa, Artificial Sequence
  Synthetic peptide
SSQNTV

SEQ ID NO: 780 - protein, 6 aa, Artificial Sequence
  Synthetic peptide
SSQQSV

SEQ ID NO: 781 - protein, 6 aa, Artificial Sequence
  Synthetic peptide
SSQQTQ

SEQ ID NO: 782 - protein, 6 aa, Artificial Sequence
  Synthetic peptide
SSQQTV

SEQ ID NO: 783 - protein, 6 aa, Artificial Sequence
  Synthetic peptide
SSQSTQ

SEQ ID NO: 784 - protein, 6 aa, Artificial Sequence
  Synthetic peptide
SSQTSV

SEQ ID NO: 785 - protein, 6 aa, Artificial Sequence
  Synthetic peptide
SSQVNS

SEQ ID NO: 786 - protein, 6 aa, Artificial Sequence
  Synthetic peptide
SSQVSN

SEQ ID NO: 787 - protein, 6 aa, Artificial Sequence
  Synthetic peptide
SSQVSQ

SEQ ID NO: 788 - protein, 6 aa, Artificial Sequence
  Synthetic peptide
SSQVSS

SEQ ID NO: 789 - protein, 6 aa, Artificial Sequence
  Synthetic peptide
SSQVST

SEQ ID NO: 790 - protein, 6 aa, Artificial Sequence
  Synthetic peptide
SSQVTG

SEQ ID NO: 791 - protein, 6 aa, Artificial Sequence
  Synthetic peptide
SSQVTQ

SEQ ID NO: 792 - protein, 6 aa, Artificial Sequence
  Synthetic peptide
SSQVTS

SEQ ID NO: 793 - protein, 6 aa, Artificial Sequence
  Synthetic peptide
SSQVTT

SEQ ID NO: 794 - protein, 6 aa, Artificial Sequence
  Synthetic peptide
SSSNAV

SEQ ID NO: 795 - protein, 6 aa, Artificial Sequence
  Synthetic peptide
SSSNQV

SEQ ID NO: 796 - protein, 6 aa, Artificial Sequence
  Synthetic peptide
SSSNSV

SEQ ID NO: 797 - protein, 6 aa, Artificial Sequence
  Synthetic peptide
SSSQAQ

SEQ ID NO: 798 - protein, 6 aa, Artificial Sequence
  Synthetic peptide
SSSQNQ

SEQ ID NO: 799 - protein, 6 aa, Artificial Sequence
  Synthetic peptide
SSSQQQ

SEQ ID NO: 800 - protein, 6 aa, Artificial Sequence
  Synthetic peptide
SSSQQT

SEQ ID NO: 801 - protein, 6 aa, Artificial Sequence
  Synthetic peptide
SSSQQV

SEQ ID NO: 802 - protein, 6 aa, Artificial Sequence
  Synthetic peptide
SSSQSQ

SEQ ID NO: 803 - protein, 6 aa, Artificial Sequence
  Synthetic peptide
SSSQSV

SEQ ID NO: 804 - protein, 6 aa, Artificial Sequence
  Synthetic peptide
SSSSGQ

SEQ ID NO: 805 - protein, 6 aa, Artificial Sequence
  Synthetic peptide
SSSSGV

SEQ ID NO: 806 - protein, 6 aa, Artificial Sequence
  Synthetic peptide
SSSSNV

SEQ ID NO: 807 - protein, 6 aa, Artificial Sequence
  Synthetic peptide
SSSSQQ

SEQ ID NO: 808 - protein, 6 aa, Artificial Sequence
  Synthetic peptide
SSSSQS

SEQ ID NO: 809 - protein, 6 aa, Artificial Sequence
  Synthetic peptide
SSSTAV

SEQ ID NO: 810 - protein, 6 aa, Artificial Sequence
  Synthetic peptide
SSSTNV

SEQ ID NO: 811 - protein, 6 aa, Artificial Sequence
  Synthetic peptide
SSSTSV

SEQ ID NO: 812 - protein, 6 aa, Artificial Sequence
  Synthetic peptide
SSSVAQ

SEQ ID NO: 813 - protein, 6 aa, Artificial Sequence
  Synthetic peptide
SSSVAS

SEQ ID NO: 814 - protein, 6 aa, Artificial Sequence
  Synthetic peptide
SSSVGG

SEQ ID NO: 815 - protein, 6 aa, Artificial Sequence
  Synthetic peptide
SSSVGQ

SEQ ID NO: 816 - protein, 6 aa, Artificial Sequence
  Synthetic peptide
SSSVGS

SEQ ID NO: 817 - protein, 6 aa, Artificial Sequence
  Synthetic peptide
SSSVNG

SEQ ID NO: 818 - protein, 6 aa, Artificial Sequence
  Synthetic peptide
SSSVNS

SEQ ID NO: 819 - protein, 6 aa, Artificial Sequence
  Synthetic peptide
SSSVNT

SEQ ID NO: 820 - protein, 6 aa, Artificial Sequence
  Synthetic peptide
SSSVQG

SEQ ID NO: 821 - protein, 6 aa, Artificial Sequence
  Synthetic peptide
SSSVQN

SEQ ID NO: 822 - protein, 6 aa, Artificial Sequence
  Synthetic peptide
SSSVQS

SEQ ID NO: 823 - protein, 6 aa, Artificial Sequence
  Synthetic peptide
SSSVQT

SEQ ID NO: 824 - protein, 6 aa, Artificial Sequence
  Synthetic peptide
SSSVSG

SEQ ID NO: 825 - protein, 6 aa, Artificial Sequence
  Synthetic peptide
SSSVSN

SEQ ID NO: 826 - protein, 6 aa, Artificial Sequence
  Synthetic peptide
SSSVSQ

SEQ ID NO: 827 - protein, 6 aa, Artificial Sequence
  Synthetic peptide
SSSVSS

SEQ ID NO: 828 - protein, 6 aa, Artificial Sequence
  Synthetic peptide
SSSVST

SEQ ID NO: 829 - protein, 6 aa, Artificial Sequence
  Synthetic peptide
STNGSV

SEQ ID NO: 830 - protein, 6 aa, Artificial Sequence
  Synthetic peptide
STNSGV

SEQ ID NO: 831 - protein, 6 aa, Artificial Sequence
  Synthetic peptide
STNSNV

SEQ ID NO: 832 - protein, 6 aa, Artificial Sequence
  Synthetic peptide
STNTNV

SEQ ID NO: 833 - protein, 6 aa, Artificial Sequence
  Synthetic peptide
STNTQV

SEQ ID NO: 834 - protein, 6 aa, Artificial Sequence
  Synthetic peptide
STNVGS

SEQ ID NO: 835 - protein, 6 aa, Artificial Sequence
  Synthetic peptide
STNVNG

SEQ ID NO: 836 - protein, 6 aa, Artificial Sequence
  Synthetic peptide
STNVNS

SEQ ID NO: 837 - protein, 6 aa, Artificial Sequence
  Synthetic peptide
STNVSG

SEQ ID NO: 838 - protein, 6 aa, Artificial Sequence
  Synthetic peptide
STNVSS

SEQ ID NO: 839 - protein, 6 aa, Artificial Sequence
  Synthetic peptide
STNVTG

SEQ ID NO: 840 - protein, 6 aa, Artificial Sequence
  Synthetic peptide
STNVTS

SEQ ID NO: 841 - protein, 6 aa, Artificial Sequence
  Synthetic peptide
STQQSG

SEQ ID NO: 842 - protein, 6 aa, Artificial Sequence
  Synthetic peptide
STQQSQ

SEQ ID NO: 843 - protein, 6 aa, Artificial Sequence
  Synthetic peptide
STQQSV

SEQ ID NO: 844 - protein, 6 aa, Artificial Sequence
  Synthetic peptide
STQQTQ

SEQ ID NO: 845 - protein, 6 aa, Artificial Sequence
  Synthetic peptide
STQQTS

SEQ ID NO: 846 - protein, 6 aa, Artificial Sequence
  Synthetic peptide
STQQTV

SEQ ID NO: 847 - protein, 6 aa, Artificial Sequence
  Synthetic peptide
STQSNV

SEQ ID NO: 848 - protein, 6 aa, Artificial Sequence
  Synthetic peptide
STQSTV

SEQ ID NO: 849 - protein, 6 aa, Artificial Sequence
  Synthetic peptide
STQTSS

SEQ ID NO: 850 - protein, 6 aa, Artificial Sequence
  Synthetic peptide
STQTSV

SEQ ID NO: 851 - protein, 6 aa, Artificial Sequence
  Synthetic peptide
STQVNS

SEQ ID NO: 852 - protein, 6 aa, Artificial Sequence
  Synthetic peptide
STQVSG

SEQ ID NO: 853 - protein, 6 aa, Artificial Sequence
  Synthetic peptide
STQVSN

SEQ ID NO: 854 - protein, 6 aa, Artificial Sequence
  Synthetic peptide
STQVST

SEQ ID NO: 855 - protein, 6 aa, Artificial Sequence
  Synthetic peptide
STQVTG

SEQ ID NO: 856 - protein, 6 aa, Artificial Sequence
  Synthetic peptide
STQVTN

SEQ ID NO: 857 - protein, 6 aa, Artificial Sequence
  Synthetic peptide
STQVTQ

SEQ ID NO: 858 - protein, 6 aa, Artificial Sequence
  Synthetic peptide
STQVTS

SEQ ID NO: 859 - protein, 6 aa, Artificial Sequence
  Synthetic peptide
STQVTT

SEQ ID NO: 860 - protein, 6 aa, Artificial Sequence
  Synthetic peptide
STSGNV

SEQ ID NO: 861 - protein, 6 aa, Artificial Sequence
  Synthetic peptide
STSNAS

SEQ ID NO: 862 - protein, 6 aa, Artificial Sequence
  Synthetic peptide
STSNGV

SEQ ID NO: 863 - protein, 6 aa, Artificial Sequence
  Synthetic peptide
STSNQV

SEQ ID NO: 864 - protein, 6 aa, Artificial Sequence
  Synthetic peptide
STSNSV

SEQ ID NO: 865 - protein, 6 aa, Artificial Sequence
  Synthetic peptide
STSQAQ

SEQ ID NO: 866 - protein, 6 aa, Artificial Sequence
  Synthetic peptide
STSQGV

SEQ ID NO: 867 - protein, 6 aa, Artificial Sequence
  Synthetic peptide
STSQNQ

SEQ ID NO: 868 - protein, 6 aa, Artificial Sequence
  Synthetic peptide
STSQQQ

SEQ ID NO: 869 - protein, 6 aa, Artificial Sequence
  Synthetic peptide
STSQQV

SEQ ID NO: 870 - protein, 6 aa, Artificial Sequence
  Synthetic peptide
STSQSQ

SEQ ID NO: 871 - protein, 6 aa, Artificial Sequence
  Synthetic peptide
STSSAV

SEQ ID NO: 872 - protein, 6 aa, Artificial Sequence
  Synthetic peptide
STSSGV

SEQ ID NO: 873 - protein, 6 aa, Artificial Sequence
  Synthetic peptide
STSSQQ

SEQ ID NO: 874 - protein, 6 aa, Artificial Sequence
  Synthetic peptide
STSSQS

SEQ ID NO: 875 - protein, 6 aa, Artificial Sequence
  Synthetic peptide
STSSQV

SEQ ID NO: 876 - protein, 6 aa, Artificial Sequence
  Synthetic peptide
STSSSQ

SEQ ID NO: 877 - protein, 6 aa, Artificial Sequence
  Synthetic peptide
STSTAV

SEQ ID NO: 878 - protein, 6 aa, Artificial Sequence
  Synthetic peptide
STSTGV

SEQ ID NO: 879 - protein, 6 aa, Artificial Sequence
  Synthetic peptide
STSTQA

SEQ ID NO: 880 - protein, 6 aa, Artificial Sequence
  Synthetic peptide
STSTQG

SEQ ID NO: 881 - protein, 6 aa, Artificial Sequence
  Synthetic peptide
STSTQT

SEQ ID NO: 882 - protein, 6 aa, Artificial Sequence
  Synthetic peptide
STSTQV

SEQ ID NO: 883 - protein, 6 aa, Artificial Sequence
  Synthetic peptide
STSTSQ

SEQ ID NO: 884 - protein, 6 aa, Artificial Sequence
  Synthetic peptide
STSTSV

SEQ ID NO: 885 - protein, 6 aa, Artificial Sequence
  Synthetic peptide
STSVAG

SEQ ID NO: 886 - protein, 6 aa, Artificial Sequence
  Synthetic peptide
STSVAN

SEQ ID NO: 887 - protein, 6 aa, Artificial Sequence
  Synthetic peptide
STSVAQ

SEQ ID NO: 888 - protein, 6 aa, Artificial Sequence
  Synthetic peptide
STSVAS

SEQ ID NO: 889 - protein, 6 aa, Artificial Sequence
  Synthetic peptide
STSVAT

SEQ ID NO: 890 - protein, 6 aa, Artificial Sequence
  Synthetic peptide
STSVGG

SEQ ID NO: 891 - protein, 6 aa, Artificial Sequence
  Synthetic peptide
STSVGN

SEQ ID NO: 892 - protein, 6 aa, Artificial Sequence
  Synthetic peptide
STSVGQ

SEQ ID NO: 893 - protein, 6 aa, Artificial Sequence
  Synthetic peptide
STSVGS

SEQ ID NO: 894 - protein, 6 aa, Artificial Sequence
  Synthetic peptide
STSVNG

SEQ ID NO: 895 - protein, 6 aa, Artificial Sequence
  Synthetic peptide
STSVNN

SEQ ID NO: 896 - protein, 6 aa, Artificial Sequence
  Synthetic peptide
STSVNS

SEQ ID NO: 897 - protein, 6 aa, Artificial Sequence
  Synthetic peptide
STSVQG

SEQ ID NO: 898 - protein, 6 aa, Artificial Sequence
  Synthetic peptide
STSVQN

SEQ ID NO: 899 - protein, 6 aa, Artificial Sequence
  Synthetic peptide
STSVQQ

SEQ ID NO: 900 - protein, 6 aa, Artificial Sequence
  Synthetic peptide
STSVQS

SEQ ID NO: 901 - protein, 6 aa, Artificial Sequence
  Synthetic peptide
STSVQT

SEQ ID NO: 902 - protein, 6 aa, Artificial Sequence
  Synthetic peptide
STSVSG

SEQ ID NO: 903 - protein, 6 aa, Artificial Sequence
  Synthetic peptide
STSVSN

SEQ ID NO: 904 - protein, 6 aa, Artificial Sequence
  Synthetic peptide
STSVSQ

SEQ ID NO: 905 - protein, 6 aa, Artificial Sequence
  Synthetic peptide
STSVSS

SEQ ID NO: 906 - protein, 6 aa, Artificial Sequence
  Synthetic peptide
STSVST

SEQ ID NO: 907 - protein, 6 aa, Artificial Sequence
  Synthetic peptide
SVNGST

SEQ ID NO: 908 - protein, 6 aa, Artificial Sequence
  Synthetic peptide
SVNGTS

SEQ ID NO: 909 - protein, 6 aa, Artificial Sequence
  Synthetic peptide
SVNQAQ

SEQ ID NO: 910 - protein, 6 aa, Artificial Sequence
  Synthetic peptide
SVNQQQ

SEQ ID NO: 911 - protein, 6 aa, Artificial Sequence
  Synthetic peptide
SVNSGT

SEQ ID NO: 912 - protein, 6 aa, Artificial Sequence
  Synthetic peptide
SVNSNQ

SEQ ID NO: 913 - protein, 6 aa, Artificial Sequence
  Synthetic peptide
SVNSNS

SEQ ID NO: 914 - protein, 6 aa, Artificial Sequence
  Synthetic peptide
SVNSTG

SEQ ID NO: 915 - protein, 6 aa, Artificial Sequence
  Synthetic peptide
SVNSTS

SEQ ID NO: 916 - protein, 6 aa, Artificial Sequence
  Synthetic peptide
SVNTGS

SEQ ID NO: 917 - protein, 6 aa, Artificial Sequence
  Synthetic peptide
SVNTSG

SEQ ID NO: 918 - protein, 6 aa, Artificial Sequence
  Synthetic peptide
SVQQSQ

SEQ ID NO: 919 - protein, 6 aa, Artificial Sequence
  Synthetic peptide
SVQQST

SEQ ID NO: 920 - protein, 6 aa, Artificial Sequence
  Synthetic peptide
SVQQTQ

SEQ ID NO: 921 - protein, 6 aa, Artificial Sequence
  Synthetic peptide
SVQQTS

SEQ ID NO: 922 - protein, 6 aa, Artificial Sequence
  Synthetic peptide
SVQQTT

SEQ ID NO: 923 - protein, 6 aa, Artificial Sequence
  Synthetic peptide
SVQSNQ

SEQ ID NO: 924 - protein, 6 aa, Artificial Sequence
  Synthetic peptide
SVQSNS

SEQ ID NO: 925 - protein, 6 aa, Artificial Sequence
  Synthetic peptide
SVQSSG

SEQ ID NO: 926 - protein, 6 aa, Artificial Sequence
  Synthetic peptide
SVQSSQ

SEQ ID NO: 927 - protein, 6 aa, Artificial Sequence
  Synthetic peptide
SVQSSS

SEQ ID NO: 928 - protein, 6 aa, Artificial Sequence
  Synthetic peptide
SVQSTG

SEQ ID NO: 929 - protein, 6 aa, Artificial Sequence
  Synthetic peptide
SVQSTQ

SEQ ID NO: 930 - protein, 6 aa, Artificial Sequence
  Synthetic peptide
SVQSTS

SEQ ID NO: 931 - protein, 6 aa, Artificial Sequence
  Synthetic peptide
SVQTSG

SEQ ID NO: 932 - protein, 6 aa, Artificial Sequence
  Synthetic peptide
SVQTSN

SEQ ID NO: 933 - protein, 6 aa, Artificial Sequence
  Synthetic peptide
SVQTSS

SEQ ID NO: 934 - protein, 6 aa, Artificial Sequence
  Synthetic peptide
SVQVSN

SEQ ID NO: 935 - protein, 6 aa, Artificial Sequence
  Synthetic peptide
SVSGNT

SEQ ID NO: 936 - protein, 6 aa, Artificial Sequence
  Synthetic peptide
SVSGQS

SEQ ID NO: 937 - protein, 6 aa, Artificial Sequence
  Synthetic peptide
SVSNAQ

SEQ ID NO: 938 - protein, 6 aa, Artificial Sequence
  Synthetic peptide
SVSNAS

SEQ ID NO: 939 - protein, 6 aa, Artificial Sequence
  Synthetic peptide
SVSNGS

SEQ ID NO: 940 - protein, 6 aa, Artificial Sequence
  Synthetic peptide
SVSNGT

SEQ ID NO: 941 - protein, 6 aa, Artificial Sequence
  Synthetic peptide
SVSNQG

SEQ ID NO: 942 - protein, 6 aa, Artificial Sequence
  Synthetic peptide
SVSNQS

SEQ ID NO: 943 - protein, 6 aa, Artificial Sequence
  Synthetic peptide
SVSNQT

SEQ ID NO: 944 - protein, 6 aa, Artificial Sequence
  Synthetic peptide
SVSNST

SEQ ID NO: 945 - protein, 6 aa, Artificial Sequence
  Synthetic peptide
SVSNTG

SEQ ID NO: 946 - protein, 6 aa, Artificial Sequence
  Synthetic peptide
SVSQAQ

SEQ ID NO: 947 - protein, 6 aa, Artificial Sequence
  Synthetic peptide
SVSQAS

SEQ ID NO: 948 - protein, 6 aa, Artificial Sequence
  Synthetic peptide
SVSQGQ

SEQ ID NO: 949 - protein, 6 aa, Artificial Sequence
  Synthetic peptide
SVSQNQ

SEQ ID NO: 950 - protein, 6 aa, Artificial Sequence
  Synthetic peptide
SVSQQG

SEQ ID NO: 951 - protein, 6 aa, Artificial Sequence
  Synthetic peptide
SVSQQQ

SEQ ID NO: 952 - protein, 6 aa, Artificial Sequence
  Synthetic peptide
SVSQQS

SEQ ID NO: 953 - protein, 6 aa, Artificial Sequence
  Synthetic peptide
SVSQSG

SEQ ID NO: 954 - protein, 6 aa, Artificial Sequence
  Synthetic peptide
SVSQSS

SEQ ID NO: 955 - protein, 6 aa, Artificial Sequence
  Synthetic peptide
SVSQTQ

SEQ ID NO: 956 - protein, 6 aa, Artificial Sequence
  Synthetic peptide
SVSSAQ

SEQ ID NO: 957 - protein, 6 aa, Artificial Sequence
  Synthetic peptide
SVSSAS

SEQ ID NO: 958 - protein, 6 aa, Artificial Sequence
  Synthetic peptide
SVSSGG

SEQ ID NO: 959 - protein, 6 aa, Artificial Sequence
  Synthetic peptide
SVSSGS

SEQ ID NO: 960 - protein, 6 aa, Artificial Sequence
  Synthetic peptide
SVSSGT

SEQ ID NO: 961 - protein, 6 aa, Artificial Sequence
  Synthetic peptide
SVSSNG

SEQ ID NO: 962 - protein, 6 aa, Artificial Sequence
  Synthetic peptide
SVSSNQ

SEQ ID NO: 963 - protein, 6 aa, Artificial Sequence
  Synthetic peptide
SVSSNS

SEQ ID NO: 964 - protein, 6 aa, Artificial Sequence
  Synthetic peptide
SVSSNT

SEQ ID NO: 965 - protein, 6 aa, Artificial Sequence
  Synthetic peptide
SVSSQG

SEQ ID NO: 966 - protein, 6 aa, Artificial Sequence
  Synthetic peptide
SVSSQQ

SEQ ID NO: 967 - protein, 6 aa, Artificial Sequence
  Synthetic peptide
SVSSQS

SEQ ID NO: 968 - protein, 6 aa, Artificial Sequence
  Synthetic peptide
SVSSQT

SEQ ID NO: 969 - protein, 6 aa, Artificial Sequence
  Synthetic peptide
SVSSQV

SEQ ID NO: 970 - protein, 6 aa, Artificial Sequence
  Synthetic peptide
SVSSSG

SEQ ID NO: 971 - protein, 6 aa, Artificial Sequence
  Synthetic peptide
SVSSSQ

SEQ ID NO: 972 - protein, 6 aa, Artificial Sequence
  Synthetic peptide
SVSSSS

SEQ ID NO: 973 - protein, 6 aa, Artificial Sequence
  Synthetic peptide
SVSTAG

SEQ ID NO: 974 - protein, 6 aa, Artificial Sequence
  Synthetic peptide
SVSTAS

SEQ ID NO: 975 - protein, 6 aa, Artificial Sequence
  Synthetic peptide
SVSTAT

SEQ ID NO: 976 - protein, 6 aa, Artificial Sequence
  Synthetic peptide
SVSTGS

SEQ ID NO: 977 - protein, 6 aa, Artificial Sequence
  Synthetic peptide
SVSTNS

SEQ ID NO: 978 - protein, 6 aa, Artificial Sequence
  Synthetic peptide
SVSTNT

SEQ ID NO: 979 - protein, 6 aa, Artificial Sequence
  Synthetic peptide
SVSTQQ

SEQ ID NO: 980 - protein, 6 aa, Artificial Sequence
  Synthetic peptide
SVSTQS

SEQ ID NO: 981 - protein, 6 aa, Artificial Sequence
  Synthetic peptide
SVSTQT

SEQ ID NO: 982 - protein, 6 aa, Artificial Sequence
  Synthetic peptide
SVSTSG

SEQ ID NO: 983 - protein, 6 aa, Artificial Sequence
  Synthetic peptide
SVSTSN

SEQ ID NO: 984 - protein, 6 aa, Artificial Sequence
  Synthetic peptide
SVSTSS

SEQ ID NO: 985 - protein, 6 aa, Artificial Sequence
  Synthetic peptide
SVSTST

SEQ ID NO: 986 - protein, 6 aa, Artificial Sequence
  Synthetic peptide
TASQSQ

SEQ ID NO: 987 - protein, 6 aa, Artificial Sequence
  Synthetic peptide
TASQVQ

SEQ ID NO: 988 - protein, 6 aa, Artificial Sequence
  Synthetic peptide
TGSNSV

SEQ ID NO: 989 - protein, 6 aa, Artificial Sequence
  Synthetic peptide
TGSNVS

SEQ ID NO: 990 - protein, 6 aa, Artificial Sequence
  Synthetic peptide
TGSSNV

SEQ ID NO: 991 - protein, 6 aa, Artificial Sequence
  Synthetic peptide
TGSVNS

SEQ ID NO: 992 - protein, 6 aa, Artificial Sequence
  Synthetic peptide
TGSVSN

SEQ ID NO: 993 - protein, 6 aa, Artificial Sequence
  Synthetic peptide
TNSGSV

SEQ ID NO: 994 - protein, 6 aa, Artificial Sequence
  Synthetic peptide
TNSGVS

SEQ ID NO: 995 - protein, 6 aa, Artificial Sequence
  Synthetic peptide
TNSQVQ

SEQ ID NO: 996 - protein, 6 aa, Artificial Sequence
  Synthetic peptide
TNSSGV

SEQ ID NO: 997 - protein, 6 aa, Artificial Sequence
  Synthetic peptide
TNSVGS

SEQ ID NO: 998 - protein, 6 aa, Artificial Sequence
  Synthetic peptide
TNSVSG

SEQ ID NO: 999 - protein, 6 aa, Artificial Sequence
  Synthetic peptide
TQSQSQ

SEQ ID NO: 1000 - protein, 6 aa, Artificial Sequence
  Synthetic peptide
TQSQVQ

SEQ ID NO: 1001 - protein, 6 aa, Artificial Sequence
  Synthetic peptide
TQSTVS

SEQ ID NO: 1002 - protein, 6 aa, Artificial Sequence
  Synthetic peptide
TQSVSN

SEQ ID NO: 1003 - protein, 6 aa, Artificial Sequence
  Synthetic peptide
TQSVSS

SEQ ID NO: 1004 - protein, 6 aa, Artificial Sequence
  Synthetic peptide
TSNGVS

SEQ ID NO: 1005 - protein, 6 aa, Artificial Sequence
  Synthetic peptide
TSNVGS

SEQ ID NO: 1006 - protein, 6 aa, Artificial Sequence
  Synthetic peptide
TSNVSG

SEQ ID NO: 1007 - protein, 6 aa, Artificial Sequence
  Synthetic peptide
TSSGNV

SEQ ID NO: 1008 - protein, 6 aa, Artificial Sequence
  Synthetic peptide
TSSGVN

SEQ ID NO: 1009 - protein, 6 aa, Artificial Sequence
  Synthetic peptide
TSSNGV

SEQ ID NO: 1010 - protein, 6 aa, Artificial Sequence
  Synthetic peptide
TSSNSV

SEQ ID NO: 1011 - protein, 6 aa, Artificial Sequence
  Synthetic peptide
TSSNVG

SEQ ID NO: 1012 - protein, 6 aa, Artificial Sequence
  Synthetic peptide
TSSQSQ

SEQ ID NO: 1013 - protein, 6 aa, Artificial Sequence
  Synthetic peptide
TSSSVQ

SEQ ID NO: 1014 - protein, 6 aa, Artificial Sequence
  Synthetic peptide
TSSVGN

SEQ ID NO: 1015 - protein, 6 aa, Artificial Sequence
  Synthetic peptide
TSSVNG

SEQ ID NO: 1016 - protein, 6 aa, Artificial Sequence
  Synthetic peptide
TSSVSG

SEQ ID NO: 1017 - protein, 6 aa, Artificial Sequence
  Synthetic peptide
TSSVSS

SEQ ID NO: 1018 - protein, 6 aa, Artificial Sequence
  Synthetic peptide
TSSVTS

SEQ ID NO: 1019 - protein, 6 aa, Artificial Sequence
  Synthetic peptide
TVNSGS

SEQ ID NO: 1020 - protein, 6 aa, Artificial Sequence
  Synthetic peptide
TVNSSG

SEQ ID NO: 1021 - protein, 6 aa, Artificial Sequence
  Synthetic peptide
TVSGNS

SEQ ID NO: 1022 - protein, 6 aa, Artificial Sequence
  Synthetic peptide
TVSGSN

SEQ ID NO: 1023 - protein, 6 aa, Artificial Sequence
  Synthetic peptide
TVSNGS

SEQ ID NO: 1024 - protein, 6 aa, Artificial Sequence
  Synthetic peptide
TVSNSG

SEQ ID NO: 1025 - protein, 6 aa, Artificial Sequence
  Synthetic peptide
TVSNST

SEQ ID NO: 1026 - protein, 6 aa, Artificial Sequence
  Synthetic peptide
TVSNVG

SEQ ID NO: 1027 - protein, 6 aa, Artificial Sequence
  Synthetic peptide
TVSQNQ

SEQ ID NO: 1028 - protein, 6 aa, Artificial Sequence
  Synthetic peptide
TVSQQV

SEQ ID NO: 1029 - protein, 6 aa, Artificial Sequence
  Synthetic peptide
TVSQSG

SEQ ID NO: 1030 - protein, 6 aa, Artificial Sequence
  Synthetic peptide
TVSQSQ

SEQ ID NO: 1031 - protein, 6 aa, Artificial Sequence
  Synthetic peptide
TVSSGN

SEQ ID NO: 1032 - protein, 6 aa, Artificial Sequence
  Synthetic peptide
TVSSNG

SEQ ID NO: 1033 - protein, 6 aa, Artificial Sequence
  Synthetic peptide
TVSSNQ

SEQ ID NO: 1034 - protein, 6 aa, Artificial Sequence
  Synthetic peptide
TVSSNS

SEQ ID NO: 1035 - protein, 6 aa, Artificial Sequence
  Synthetic peptide
TVSSSQ

SEQ ID NO: 1036 - protein, 6 aa, Artificial Sequence
  Synthetic peptide
TVSTSG

SEQ ID NO: 1037 - protein, 6 aa, Artificial Sequence
  Synthetic peptide
TVSTSN

SEQ ID NO: 1038 - protein, 6 aa, Artificial Sequence
  Synthetic peptide
TVSTSS

SEQ ID NO: 1039 - protein, 6 aa, Artificial Sequence
  Synthetic peptide
TVSTST

SEQ ID NO: 1040 - protein, 6 aa, Artificial Sequence
  Synthetic peptide
VGSTNS

SEQ ID NO: 1041 - protein, 6 aa, Artificial Sequence
  Synthetic peptide
VNSTSG

SEQ ID NO: 1042 - protein, 6 aa, Artificial Sequence
  Synthetic peptide
VNSTSN

SEQ ID NO: 1043 - protein, 6 aa, Artificial Sequence
  Synthetic peptide
VSSQSQ

SEQ ID NO: 1044 - protein, 6 aa, Artificial Sequence
  Synthetic peptide
VSSQVQ

SEQ ID NO: 1045 - protein, 6 aa, Artificial Sequence
  Synthetic peptide
VSSTNG

SEQ ID NO: 1046 - protein, 6 aa, Artificial Sequence
  Synthetic peptide
VTSNSG

SEQ ID NO: 1047 - protein, 6 aa, Artificial Sequence
  Synthetic peptide
VTSQSQ

SEQ ID NO: 1048 - protein, 6 aa, Artificial Sequence
  Synthetic peptide
VEALYL

SEQ ID NO: 1049 - protein, 6 aa, Artificial Sequence
  Synthetic peptide
LYQLEN

SEQ ID NO: 1050 - protein, 6 aa, Artificial Sequence
  Synthetic peptide
GYVIIK

SEQ ID NO: 1051 - protein, 6 aa, Artificial Sequence
  Synthetic peptide
SNQNNF

SEQ ID NO: 1052 - protein, 6 aa, Artificial Sequence
  Synthetic peptide
SVLTSL

SEQ ID NO: 1053 - protein, 6 aa, Artificial Sequence
  Synthetic peptide
SSTNVG

SEQ ID NO: 1054 - protein, 6 aa, Artificial Sequence
  Synthetic peptide
SVSSSY

SEQ ID NO: 1055 - protein, 6 aa, Artificial Sequence
  Synthetic peptide
GAILSS

SEQ ID NO: 1056 - protein, 6 aa, Artificial Sequence
  Synthetic peptide
GAIIGL

SEQ ID NO: 1057 - protein, 6 aa, Artificial Sequence
  Synthetic peptide
AIIGLM

SEQ ID NO: 1058 - protein, 6 aa, Artificial Sequence
  Synthetic peptide
GGVVIA

SEQ ID NO: 1059 - protein, 6 aa, Artificial Sequence
  Synthetic peptide
SVNDLV

SEQ ID NO: 1060 - protein, 6 aa, Artificial Sequence
  Synthetic peptide
LKVKVL

SEQ ID NO: 1061 - protein, 6 aa, Artificial Sequence
  Synthetic peptide
NKGAII